SEQUENCE LISTING (25 sequences)

SEQ ID NO: 1 - protein, 181 aa, Glycine max
DFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 2 - protein, 181 aa, Glycine max; identical to SEQ ID NO: 1

SEQ ID NO: 3 - protein, 181 aa, Glycine max; identical to SEQ ID NO: 1

SEQ ID NO: 4 - protein, 181 aa, Glycine max
DFVLDNEGNPLENGGTYYILSDITAFGGIRAAPTGNERCPLTVVQSRNELDKGIETIISSPYRIRFIAEGHPLSLKFDSFAVIMLCVGIPTEWSVVEDLPEGPAVKIGENKDAMDGWFRLERVSDDEFNNYKLVFCPQQAEDDKCGDIGISIDHDDGTRRLVVSKNKPLVVQFQKLDKESL

SEQ ID NO: 5 - protein, 210 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 6 - protein, 208 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVDCSEYPKPDCTAEDRPLCGSDNKTYGNKCNFCNAVVESNGTLTLSHFGKC

SEQ ID NO: 7 - protein, 210 aa, Gallus gallus
MAMAGVFVLFSFVLCGFLPDAVFGAEVDCSRFPNATDMEGKDVLVCNKDLRPICGTDGVTYTNDCLLCAYSVEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 8 - protein, 186 aa, Gallus gallus
AEVDCSRFPNATDKEGKDVLVCNKDLRPICGTDGVTYNNECLLCAYSIEFGTNISKEHDGECKETVPMNCSSYANTTSEDGKVMVLCNRAFNPVCGTDGVTYDNECLLCAHKVEQGASVDKRHDGECRKELAAVSVDCSEYPKPDCTAEDRPLCGSDNKTYGNKCNFCNAVVESNGTLTLSHFGKC

SEQ ID NO: 9 - protein, 208 aa, Gallus gallus; identical to SEQ ID NO: 6

SEQ ID NO: 10 - protein, 210 aa, Gallus gallus; identical to SEQ ID NO: 5

SEQ ID NO: 11 - protein, 209 aa, Meleagris gallopavo
MAMAGIFVLFSFALCGFLPDAAFGVEVDCSRFPNTTNEEGKDVLVCTEDLRPICGTDGVTHSECLLCAYNIEYGTNISKEHDGECREAVPMDCSRYPNTTNEEGKVMILCNKALNPVCGTDGVTYDNECVLCAHNLEQGTSVGKKHDGGCRKELAAVSVDCSEYPKPACTLEYRPLCGSDNKTYGNKCNFCNAVVESNGTLTLSHFGKC

SEQ ID NO: 12 - protein, 185 aa, Meleagris gallopavo
VEVDCSRFPNTTNEEGKDVLVCTEDLRPICGTDGVTHSECLLCAYNIEYGTNISKEHDGECREAVPMDCSRYPNTTSEEGKVMILCNKALNPVCGTDGVTYDNECVLCAHNLEQGTSVGKKHDGECRKELAAVSVDCSEYPKPACTLEYRPLCGSDNKTYGNKCNFCNAVVESNGTLTLSHFGKC

SEQ ID NO: 13 - protein, 208 aa, Numida meleagris
MAMAGVFVLFSFALCGFLPDAAFGVEVDCSRFPNATNEEGKDVLVCTEDLRPICGTDGVTYSNDCLLCAYNIEYGTNISKEHDGECREAVPVDCSRYPNMTSEEGKVLILCNKAFNPVCGTDGVTYDNECLLCAHNVEQGTSVGKKHDGECRKELAAVDCSEYPKPACTMEYRPLCGSDNKTYDNKCNFCNAVVESNGTLTLSHFGKC

SEQ ID NO: 14 - protein, 171 aa, Anas platyrhynchos
QVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVSVDCSGYPKPACTMEYMPLCGSDNKTYGNKCNFCNAVV

SEQ ID NO: 15 - protein, 77 aa, Anas platyrhynchos
MRTAGVVVLLALALCCCPDTVALKGYCSNYVVPRNFCTLEYIPHCGSDGVTYANKCLFCNAFLRSRRTLRLLYLREC

SEQ ID NO: 16 - protein, 54 aa, Anas platyrhynchos
VATVDCSGYPKPACTMEYMPLCGSDNKTYGNKCNFCNAVVDSNGTLTLSHFGEC

SEQ ID NO: 17 - protein, 54 aa, Anas platyrhynchos; identical to SEQ ID NO: 16

SEQ ID NO: 18 - protein, 226 aa, Anas platyrhynchos
MVLSVLPPFTQVTLAQGHGALTCCPRHPETCPELERSTSWRGIKVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 19 - protein, 245 aa, Anas platyrhynchos
MISTSSGQLQDNISCITSKLPSLVLPPFTQVTLAQGHGALTCCPRHPETCPELERSTSWRGIKVDCSRFPNTTNEEGKEVLLCTKELSPVCGTDGVTYSNECLLCAYNIEYGTNISKDHDGECKEAVPADCSMYPNMTNEEGKMTLLCNKMFSPVCGTDGVTYDNECMLCAHNVEQGTSVGKKYDGKCKKEVATVDCSGYPKPACTMEYMPLCGSDNKTYGNKCNFCNAVVDSNGTLTLSHFGEC

SEQ ID NO: 20 - protein, 42 aa, Glycine max
DDESSKPCCDQCACTKSNPPQCRCSDMRLNSCHSACKSCICA

SEQ ID NO: 21 - protein, 35 aa, Glycine max
KSCICALSYPAQCFCVDITDFCYEPCKPSEDDKEN

SEQ ID NO: 22 - protein, 110 aa, Glycine max
MVVLKVCLVLLFLVGGTTSANLRLSKLGLLMKSDHQHSNDDESSKPCCDQCACTKSNPPQCRCSDMRLNSCHSACKSCICALSYPAQCFCVDITDFCYEPCKPSEDDKEN

SEQ ID NO: 23 - protein, 118 aa, Glycine max
MGLKNNMVVLKVCLVLLFLVGGTTSANLRLSKLGLLMKSDHHQHSNDDESSKPCCDQCACTKSNPPQCRCSDMRLNSCHSACKSCICALSYPAQCFCVDITDFCYEPCKPSEDDKENY

SEQ ID NO: 24 - protein, 217 aa, Glycine max
MKSTIFFLFALFCAFTTSYLPSAIADFVLDNEGNPLENGGTYYILSDITAFGGIRAAPTGNERCPLTVVQSRNELDKGIGTIISSPYRIRFIAEGHPLSLKFDSFAVIMLCVGIPTEWSVVEDLPEGPAVKIGENKDAMDGWFKLERVSDDEFNNYKLVFCPQQAEDDKCGDIGISIDHDDGTRRLVVSKNKPLVVQFQKLDKESLAKKNHGLSRSE

SEQ ID NO: 25 - protein, 217 aa, Glycine soja
MKSTIFFALFLFCAFTTSYLPSAIADFVLDNEGNPLENGGTYYILSDITAFGGIRAAPTGNERCPLTVVQSRNELDKGIGTIISSPYRIRFIAEGHPLSLKFDSFAVIMLCAGIPTEWSVVEDLPEGPAVKIGENKDAMDGWFRLERVSDDEFNNYKLVFCPQQAEDDKCGDIGISIDHDDGTRRLVVSKNKPLVVQFQKLDKESLAKKNHGLSRSE

(19) United States
(12) Patent Application Publication
(10) Pub. No.: US 2022/0362303 A1
(43) Pub. Date: Nov. 17, 2022

(54) STABILIZED COLOSTRUM COMPOSITIONS

(71) Applicant: PanTheryx, Inc., Phoenix, AZ (US)

(72) Inventors: Raymond John Playford, Cobham (GB); Michael James Weiser, Erie, CO (US); Tania Marchbank, Epsom (GB); Sandra Johanna Maria ten Bruggencate, Ede (NL); George Stagnitti, Longmont, CO (US)

(73) Assignee: PanTheryx, Inc., Phoenix, AZ (US)

(21) Appl. No.: 17/681,166

(22) Filed: Feb. 25, 2022

Related U.S. Application Data
(60) Provisional application No. 63/154,495, filed on Feb. 26, 2021.

(51) Int. Cl. A61K 35/20; A61K 38/17; A61K 35/12; A61K 38/18; A61P 1/00; A61K 36/48; A61K 47/36; A61K 38/57; A61K 39/39; A61K 47/14; A23L 33/115; A23L 33/125; A23L 33/19; A23L 33/00

(52) U.S. Cl. CPC A61K 35/20; A61K 38/1709; A61K 35/12; A61K 38/1808; A61P 1/00; A61K 36/48; A61K 47/36; A61K 38/57; A61K 39/39; A61K 47/14; A23L 33/115; A23L 33/125; A23L 33/19; A23L 33/40; A61K 2039/55588

(57) ABSTRACT

Compositions are provided comprising a pro-reparative factor such as a colostrum, a nutritional protease inhibitor to decrease enzymatic digestion of the pro-reparative factors, and optionally a stabilizing agent, to allow the pro-reparative factors to reach the small intestine intact following oral administration. Methods are provided for treating, alleviating, and preventing inflammatory bowel disease (IBD) and other disorders of the gastrointestinal tract.

### CLAIM OF PRIORITY

This application claims the benefit of priority to U.S. Provisional Application No. 63/154,495, titled “Stabilized Colostrum Compositions,” filed Feb. 26, 2021, which is incorporated herein by reference in its entirety.

### SEQUENCE LISTING

The present application includes a Sequence Listing which has been submitted electronically in ASCI format and is hereby incorporated by reference in its entirety. Said ASCI copy, entitled “Sequence-Listing-40735-0056USP1” was created on Feb. 26, 2021 and filed with U.S. Provisional Application No. 63/154,495 and has a size of 36.4 kilobytes (KB).

### FIELD

Embodiments provided herein relate to stabilized colostrum compositions and methods of using the same.

### BACKGROUND

Inflammatory bowel disease (IBD) is a severe relapsing condition predominantly affecting the gastrointestinal tract and is subdivided into ulcerative colitis (UC) affecting the large intestine and Crohn's disease which may affect the entire gastrointestinal tract, with a predilection for the terminal ileum. In both conditions, an excess inflammatory response leads to disruption of mucosal integrity, ingress of luminal contents into the mucosa and bloodstream and further exacerbation of the inflammatory process. Current therapies involve powerful immune modulators with serious side effect such as prednisolone, azathioprine or monoclonal antibodies directed against the inflammatory cascade (e.g., TNFα) with associated risks of serious infections and/or bone marrow suppression. Novel therapies are therefore required.

Colostrum is the first milk produced during the initial few days after birth. Compared to the milk subsequently produced, it is particularly rich in immunoglobulins, antimicrobial peptides (e.g. lactoferrin, lactoperoxidase) and other bioactive molecules, including growth factors.

Bovine colostrum (BC) may be useful for the prevention and treatment of a variety of gastrointestinal conditions but the degradation of colostrum in the gastrointestinal tract hinders its utility. There remains a need for stabilized colostrum compositions and safe, effective, and economical methods for treating, alleviating, preventing, and/or preventing relapse of conditions affecting the gastrointestinal tract including inflammatory bowel disease (IBD), for example, ulcerative colitis (UC) affecting the large intestine, or Crohn's disease which may affect the entire gastrointestinal tract, with a predilection for the terminal ileum. There also is a need for stabilized colostrum compositions for use in dietary supplementation, in functional foods and beverages, and for supporting normal gastrointestinal and immune function.

### SUMMARY

The disclosure provides stabilized colostrum compositions. The compositions may include a pro-reparative factor; a nutritional serine protease inhibitor; and optionally a nutraceutically acceptable carrier or excipient.

The disclosure provides compositions including a pro-reparative factor, a stabilizing agent, and optionally a nutraceutically acceptable carrier or excipient.

The disclosure provides a method for treating, alleviating, or preventing a disease or disorder associated with inflammation of the gastrointestinal tract in a subject in need thereof, comprising administering a composition to the subject, the composition comprising: a therapeutically effective amount of a pro-reparative factor, an immune factor, or a combination thereof; a nutritional serine protease inhibitor; and optionally a nutraceutically acceptable carrier or excipient. The nutritional serine protease inhibitor may be a nutritional trypsin inhibitor. In some embodiments, the composition may further comprise a stabilizing agent.

The disclosure provides a method of treating, alleviating, or preventing a disease or disorder associated with inflammation of the gastrointestinal tract in a subject in need thereof is provided, comprising administering to the subject, the composition comprising: a therapeutically effective amount of a pro-reparative factor and/or an immune factor; a stabilizing agent; and optionally a nutraceutically acceptable carrier or excipient.

The disclosure provides a method of enhancing a vaccination antibody titer in a subject, comprising orally administering a therapeutically effective amount of a composition to the subject. The composition may comprise a pro-reparative factor, an immune factor, or a combination thereof; a nutritional serine protease inhibitor; and optionally a nutraceutically acceptable carrier or excipient; and optionally a stabilizing agent. The method of enhancing a vaccination antibody titer in a subject comprising administering a vaccine to the subject.

The disclosure provides for a composition comprising a purified lactoferrin, a stabilizing agent, and optionally a nutraceutically acceptable carrier or excipient.

The present disclosure is based on, inter alia, the surprising discovery that addition of certain combination products results in a synergistic protection of colostrum from peptic digestion, suggesting that compositions comprising colostrum and the combination products provide a more beneficial oral bioavailability profile.

### BRIEF DESCRIPTION OF THE DRAWINGS

FIG. 1A depicts a schematic representation of an in vitro sample enzymatic digestion protocol.

FIG. 1B depicts a bar graph showing effect of protease inhibitors on growth factor preservation in vitro via an Alamar bluecell proliferation assay in human AGS cell line.

FIG. 1C shows a bar graph of effect of protease inhibitors on growth factor preservation in vitro via an Alamar blue cell proliferation assay in human AGS cell line.

FIG. 1D shows a bar graph of effect of combination products on in vitro stability of BC. Stability measured via Alamar blue cell proliferation assay.

FIG. 1E depicts a bar graph showing effect of combination products on in vitro stability of BC. Stability measured via bovine IgG E. coli binding.

FIG. 2A depicts a bar graph showing the influence of combination products on IgG immunoreactivity via ELISA.

FIG. 2B depicts a bar graph showing the influence of combination products on growth factor immunoreactivity. TGFβ immunoreactivity measured via ELISA.

FIG. 2C depicts a bar graph showing the influence of combination products on growth factor immunoreactivity. EGF immunoreactivity measured via ELISA.

FIG. 2D depicts a bar graph showing the influence of combination products on growth factor immunoreactivity. Lactoferrin immunoreactivity measured via ELISA.

FIG. 3A depicts a bar graph of rat cumulative total body weight change over 9 days and influence of serine protease inhibitors on protective activity of BC+egg or EGF in DSS-induced colitis in rats.

FIG. 3B depicts a bar graph of cumulative Disease Activity Index (DAI) score in DSS-induced colitis in rats over 9 days and influence of serine protease inhibitors on protective activity of BC+egg or EGF.

FIG. 3C depicts a bar graph of colonic tissue Myeloperoxidase (MPO) concentrations in DSS-induced colitis in rats over 9 days and influence of serine protease inhibitors on activity of BC+egg or EGF.

FIG. 3D depicts a bar graph of total histological colitis score in rats in DSS-induced colitis model.

FIGS. 4A-4H show photomicrographs of morphology in DSS-induced colitis in same rats as FIGS. 2A-2D. More specifically:

FIG. 4A shows a photomicrograph of normal (no DSS) rat crypt structure.

FIG. 4B shows a photomicrograph of rat colon after administration of DSS over 7 d.

FIG. 4C shows a photomicrograph of rat colon after treatment with serine protease inhibitor ovomucoid alone.

FIG. 4D shows a photomicrograph of rat colon after treatment with serine protease inhibitor SBTI alone.

FIG. 4E shows a photomicrograph of rat colon in an animal that received BC+egg.

FIG. 4F shows a photomicrograph of rat colon in an animal that received the combination of BC+egg+ovomucoid.

FIG. 4G shows a photomicrograph of rat colon after treatment with EGF alone.

FIG. 4H shows a photomicrograph of rat colon in an animal that received the combination of EGF+SBTI.

FIG. 5A depicts a bar graph of total body weight gain for mice receiving the specified treatments over 7 days.

FIG. 5B shows a bar graph of cumulative Disease Activity Index (DAI) scores determined for mice receiving the specified treatments.

FIG. 5C shows a bar graph of final Disease Activity Index (DAI) scores determined for mice receiving the specified treatments.

FIG. 6A depicts a bar graph of myeloperoxidase (MPO) activity in colonic tissue of the mice in DSS-induced colitis model.

FIG. 6B shows a bar graph of histological score per colon in DSS-induced colitis model of FIG. 6A. Representative photomicrographs are shown in FIGS. 7A-7G.

FIG. 7A shows a representative photomicrograph of colonic tissue of the “No DSS control” mice in the DSS-induced colitis model.

FIG. 7B shows a representative photomicrograph of colonic tissue of the “DSS alone” mice in the DSS-induced colitis model.

FIG. 7C shows a representative photomicrograph of colonic tissue of the “DSS+C7 7 mg/kg” mice in the DSS-induced colitis model.

FIG. 7D shows a representative photomicrograph of colonic tissue of the “DSS+C7 20 mg/kg” mice in DSS-induced colitis model.

FIG. 7E shows a representative photomicrograph of colonic tissue of the “DSS+C12 7 mg/kg” mice in DSS-induced colitis model.

FIG. 7F shows a representative photomicrograph of colonic tissue of the “DSS+C12 20 mg/kg” mice in DSS-induced colitis model.

FIG. 7G shows a representative photomicrograph of colonic tissue of the “DSS+C17 20 mg/kg” mice in DSS-induced colitis model.

FIG. 8 depicts a bar graph of proliferative effect of the specified groups in either acid resistant capsules or in comparative non-acid resistant conventional capsules.

### DETAILED DESCRIPTION OF THE DISCLOSURE

### Definitions

The terminology used herein is for the purpose of describing particular embodiments only and is not intended to be limiting of the disclosure. Unless otherwise defined, all terms, including technical and scientific terms used in the description, have the same meaning as commonly understood by one of ordinary skill in the art to which this disclosure belongs. In the event of conflicting terminology, the present specification is controlling

As used in the specification and the appended claims, the singular forms “a”, “an” and “the” include plural references unless the content clearly dictates otherwise. Thus, for example, a composition containing “a pro-reparative factor” includes a mixture of two or more pro-reparative factors.

The term “or” is generally employed in its sense including “and/or”, which is intended to encompasses any and all possible combinations of one or more of the associated listed items, unless the context clearly dictates otherwise.

The term “about,” as used herein when referring to a measurable value such as an amount, a temporal duration, and the like, is meant to encompass variations of ±20%, or ±10%, or ±5%, or ±1%, or ±0.5%, or ±0.1% from the specified value, as such variations are appropriate to perform the disclosed methods.

The term “additional active agent” refers to an agent useful either alone, administered simultaneously, administered sequentially, or in combination with one or more additional agents, in the treatment, prophylaxis or palliative care of a subject afflicted with a disease or disorder. An additional active agent can be, for example, an antibiotic, antifungal, antiviral, antimicrobial, antiparasitic, micronutrient, oral rehydration salt, antidiarrheal adsorbant, anticholinergic drug, antisecretory agent, antimotility drug, or colostrum component, or a combination thereof. An additional active agent can also be a histamine type 2 receptor blocker or a proton pump inhibitor. A histamine type 2 receptor blocker can be ranitidine, famotidine, nizatidine, or cimetidine. A proton pump inhibitor can be omeprazole, lansoprazole, rabeprazole, pantoprazole, esomeprazole, or dexlansoprazole. An additional active agent can be co-administered with any of the compositions disclosed herein. In some embodiments, an additional active agent can be employed in any of the compositions disclosed herein in an amount previously employed alone as a “standard of care.” In some embodiments, an additional active agent can be employed in the compositions in less than an amount previously employed alone as a “standard of care.” In some embodiments, an additional active agent can be co-administered or employed in any composition described herein in an amount effective to cause measurable reduction of a symptom or sign of a disease, disorder, or condition related to inflammation or damage of the gastrointestinal tract, decreased enteric inflammation, change in intestinal microbiome, decreased blunting of intestinal villi, increased intestinal integrity, decreased ulceration, decreased leakage of intestinal contents, decreased systemic inflammation, significant change in a biomarker, reduction of diarrhea volume, reduction of diarrhea duration, reduction of abdominal pain, reduction of nausea, reduction of cramping, reduction of loss of bowel control or urgency of diarrhea symptoms, or in an amount to increase physician-assessed well-being of the subject; for example, as compared to the composition without the additional active agent.

The term “agglomeration” or “agglomerating” refers to a technique used to increase the particle size of fine powders. The increase in particle size improves the wettability and solubility and therefore gives instant properties to the compositions of the disclosure. The process can involve rewetting the fine, spray-dried powder, followed by a second drying cycle resulting in an “agglomerate.”

The term “colostrum” refers to first milk obtained from a mammal up to 24 hrs, 48 hrs, or 72 hrs (e.g., about 2, 5, 10, 12, 24, 36, 48 or 72 hours) after parturition, (i.e., giving birth). Colostrum can be obtained from a cow, sheep, goat, buffalo, or other mammal. In some embodiments, the colostrum is bovine colostrum. In some embodiments, the colostrum is not human colostrum. The term “late colostrum” refers to colostrum obtained between 72 hrs and 7 days after parturition (e.g., about 3, 4, 5, 6, or 7 days after parturition).

In understanding the scope of the present disclosure, the term “comprising” and its derivatives, as used herein, are intended to be open ended terms that specify the presence of the stated features, elements, components, groups, integers, operations, and/or steps, but do not exclude the presence of other unstated features, elements, components, groups, integers, operations and/or steps. The foregoing also applies to words having similar meanings such as the terms “including”, “having” and their derivatives. In embodiments or claims where the term comprising is used as the transition phrase, such embodiments can also be envisioned with replacement of the term “comprising”, “including”, “having” and their derivatives with the terms “consisting of” or “consisting essentially of” The term “consisting” and its derivatives are intended to be close ended terms that specify the presences of stated features, elements, components, groups, integers, and/or steps. The term “consisting essentially of”, as used herein, is intended to specify the presence of the stated features, elements, components, groups, integers, and/or steps as well as those that do not materially affect the basic and novel characteristics of these features, elements, components, groups, integers, and/or steps. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. Any single term, single element, single phrase, group of terms, group of phrases, or group of elements described herein can each be specifically excluded from the claims. Finally, terms of degree such as “substantially,” “about,” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The terms “egg” or “egg product” each mean an avian sourced whole shell egg (conventional, immunized or otherwise) or any product or fraction derived therefrom. The avian can be, for example, a hen of one or more avian domestic species selected from chicken, duck, goose, turkey, guineafowl, pigeon, quail, emu, or ostrich.

The term “immune factor” as used herein refers to peptides and other factors that regulate immune responses. An immune factor can be, but is not limited to, an immunoglobulin, a cytokine, or an anti-microbial peptide. The term “immunoglobulin” as used herein refers to a class of proteins present in the serum and cells of the immune system, which functions as an antibody. An immunoglobulin can be, but is not limited to, an IgG, IgA, or IgM. The term “cytokine” as used herein refers to any of a number of substances which are secreted by cells of the immune system and have an effect on other cells. A cytokine may be, but is not limited to, interleukins (e.g., IL-1β, IL-2, IL-6, IL-10, IL-17), tumor necrosis factor α, interferon γ, granulocyte, macrophage, or granulocyte macrophage colony stimulating factors. The term “anti-microbial peptide” as used herein refers to naturally occurring molecules that are produced as a first line of defense by all multicellular organisms. These proteins can have broad activity to directly kill bacteria, yeasts, fungi, viruses, and even cancer cells. An anti-microbial peptide can be, but is not limited to, lactoferrin, lactoperoxidase or lysozyme.

The term “including” as used herein is non-limiting in scope, such that additional elements are contemplated as being possible in addition to those listed; this term may be read in any instance as “including, but not limited to.”

The alleviation or improvement of the sign or symptom or biomarker may occur following administering the composition of a period of time from 1 week to 2 years, 2 weeks to 18 months, 3 weeks to 12 months, 4 weeks to 9 months, 6 weeks to 6 months, or 8 weeks to 3 months, or 1, 2, 3, 4, 5, 6, 7, 8, 9, 10, 11, 12, 13, 14, 15, 16, 17, 18, 19, 20, 21, 22, 23, or 24 months after starting the administration.

An “effective amount” of a component or composition may be further defined herein as an amount effective to provide a desirable activity, or to improve or preserve the stability of a component of the composition or the stability of the composition by at least 10%, 20%, 30%, 50%, 70%, 100% or 2-fold, 3-fold, 4-fold, or greater, for example, relative to the same component or composition without the agent when exposed to the same conditions for the same period of time. In some embodiments, “effective amount” and “therapeutically effective amount” are used interchangeably, and refer to an amount of a compound, formulation, material, component, or composition as described herein effective to achieve a particular biological result or provide a therapeutic or prophylactic benefit. Such results can include, but are not limited to, an amount when administered to a mammal, causes a reduction in myeloperoxidase (MPO) in a patient sample. The patient sample can be a tissue, blood, serum, plasma, saliva, or stool sample. In some embodiments, the effective amount of the composition can reduce fecal MPO in the subject. The effective amount of the composition can reduce MPO in a colonic or rectal tissue sample. A reduction in MPO can be a reduction of about 0.1, 1, 5, 10, 20, 30, 40, 50% or more. The effective amount of the composition can be from about 0.5 g to 100 g, 1 g to 75 g, 2 g to 50 g, 3 g to 20 g, or at least 1 gram, 2 grams, 3 grams, 4 grams, 5 grams, 6 grams, 7 grams, 8 grams, 9 grams, 10 grams on a dry weight basis per daily therapeutic dose of composition. A composition can be in a dry, powdered, semi-solid, solid, paste, or liquid form.

The term “nutraceutically acceptable carrier or excipient” refers to a non-toxic, inert solid, semi-solid or liquid filler, carrier, diluent, excipient, vehicle, encapsulating material, or formulation auxiliary of any type. Some examples of materials that can serve as nutraceutically acceptable carriers are provided herein and include, for example, sugars such as lactose, glucose and sucrose; cyclodextrins such as alpha- (α), beta- (β) and gamma- (γ) cyclodextrins; starches such as corn starch, potato starch; partially hydrolyzed starch such as dextrin; cellulose and its derivatives such as sodium carboxymethyl cellulose, ethyl cellulose, cellulose acetate; powdered tragacanth; malt; gelatin; talc; excipients such as silicon dioxide, cocoa butter, suppository waxes; vegetable oils such as peanut oil, cottonseed oil, safflower oil, sesame oil, olive oil, corn oil, and soybean oil; medium-chain triglycerides, e.g. extracted from palm kernel oil or coconut oil; glycols such as propylene glycol; esters such as ethyl oleate and ethyl laurate; agar; buffering agents such as magnesium hydroxide, aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as solubilizing agents, coloring agents, releasing agents, coating agents, sweetening, flavoring, perfuming agents, preservatives and antioxidants can also be present in the composition, according to the judgment of the formulator. A flavoring can be a natural flavoring.

The terms, “patient”, “subject” or “subjects” include but are not limited to humans, the term can also encompass other mammals, or domestic or exotic animals, for example, pigs, horses, sheep, cattle, oxen, goats, alpaca, llama, camel, dogs, cats, ferrets, rabbits, or birds. In some embodiments, the subject is a human subject. A human subject can be a neonate subject 28 days of age or less, a non-neonate subject older than 28 days, a juvenile subject less than 18 years of age, an adult subject 18 years of age or older, or a senior subject 60 years of age or older. In a specific embodiment, the subject is a non-neonate human subject. In other embodiments, the subject is a domestic animal. In some embodiments, the domestic animal can be a neonate domestic animal, such as a calf, piglet, kid, lamb, or foal. In some embodiments, the domestic animal can be a pregnant or nursing female domestic animal. In some embodiments, the domestic animal can be a production animal. A production animal can be a dairy cow, dairy goat, beef cattle, hogs, poultry, wool producing animal such as a sheep, alpaca, angora rabbit, camel, goat, llama, musk oxen. In some embodiments, a domestic animal can be a companion animal such as a horse, dog, cat, ferret, or rabbit. In some embodiments, a domestic animal can be a performance animal such as a race horse, performance horse, show horse, herding dog, or hunting dog.

The term “pro-reparative factor” refers to small molecules and peptides such as growth factors that influence cell growth, differentiation, development, and function. Such pro-reparative factors include, but are not limited to, Epidermal Growth Factor (EGF), Transforming Growth Factor α (TGF-α), Transforming Growth Factor β (TGF-β), Insulin-Like Growth Factor I (IGF-I), Insulin-Like Growth Factor II (IGF-II), and Platelet-derived growth factor (PDGF). The term pro-reparative factor can refer to a single factor, such as an isolated pro-reparative peptide, or a group of factors, or a mixture comprising pro-reparative factors, such as colostrum.

The term “therapeutically effective amount” refers to an amount of a compound, formulation, material, component, or compositions as described herein effective to achieve a particular biological result or provides a therapeutic or prophylactic benefit. Such results can include, but are not limited to, improved treatment, healing, prevention, or amelioration of a disease, disorder, or side effect, or a decrease in the rate of advancement of a disease or disorder. Such results may include, but are not limited to, a reduction in MPO in a patient sample. The skilled artisan would understand that the “therapeutically effective amount” can vary depending on the compound, the disease and its severity, route of administration, and the condition, age, weight, gender etc. of the subject to be treated, and the like.

The terms “treating” or “treatment” of a disease state or condition includes: (i) preventing the disease state or condition, i.e., causing one or more of the clinical symptoms of the disease state or condition not to develop in a subject that may be exposed to or predisposed to the disease state or condition, but does not yet experience or display symptoms of the disease state or condition, (ii) inhibiting or alleviating the disease state or condition, i.e., arresting the development of the disease state or condition or one or more of its clinical symptoms, (iii) relieving the disease state or condition, i.e., causing temporary or permanent regression of the disease state or condition or one or more of its clinical symptoms, or (iv) reducing the frequency or severity of at least one sign or symptom of a disease or disorder experienced by a subject.

All pronouns are intended to be given their broadest meaning. Unless stated otherwise, female pronouns encompass the male, male pronouns encompass the female, singular pronouns encompass the plural, and plural pronouns encompass the singular.

Throughout this disclosure, various aspects of the embodiments can be presented in a range format. It should be understood that the description in range format is merely for convenience and brevity and should not be construed as an inflexible limitation. Accordingly, the description of a range should be considered to have specifically disclosed all the possible subranges as well as individual numerical values within that range. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, etc., as well as individual numbers within that range, for example 1, 2, 2.3, 3, 4, 5, 5.7, 6, 7, 8, 8.9, 9, 9.5, 9.9, and 10. This applies regardless of the breadth of the range. Unless otherwise explicitly stated to the contrary, a range that is disclosed also includes the endpoints of the range.

The term “substantial identity” or “substantially identical” is meant to convey a polypeptide or nucleic acid molecule exhibiting at least 50% identity to a reference amino acid sequence (for example, any one of the amino acid sequences described herein) or nucleic acid sequences (for example, any one of the nucleic acid sequences described herein). In some embodiments a sequence can be, for example, at least 60%, 80%, 85%, 90%, 95%, 99% or more identical at the amino acid level or nucleic acid level to the sequences used for comparison.

Sequence identity can be measured/determined using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program can be used, with a probability score between e3 and e100 indicating a closely related sequence. In some embodiments, sequence identity is determined by using BLAST with the default settings.

To the extent embodiments provided for herein, includes composition comprising various proteins, these proteins may, in some instances, comprise amino acid sequences that have sequence identity to the amino acid sequences disclosed herein. Therefore, in certain embodiments, depending on the particular sequence, the degree of sequence identity can be greater than about 50% (e.g. 50%, 60%, 70%, 75%, 80%, 85%, 90%, 91%, 92%, 93%, 94%, 95%, 96%, 97%, 98%, 99%, or more) to the SEQ ID NOs disclosed herein. These proteins can include homologs, orthologues, allelic variants and functional mutants. Typically, 50% identity or more between two polypeptide sequences is considered to be an indication of functional equivalence. Identity between polypeptides can be determined by the Smith-Waterman homology search algorithm as implemented in the MPSRCH program (Oxford Molecular), using an affine gap search with parameters gap open penalty−12 and gap extension penalty=1.

These proteins can, compared to the disclosed proteins, include one or more (e.g. 1, 2, 3, 4, 5, 6, 7, 8, 9, 10, etc.) conservative amino acid replacements i.e. replacements of one amino acid with another which has a related side chain. Genetically-encoded amino acids are generally divided into four families: (1) acidic i.e. aspartate, glutamate; (2) basic i.e. lysine, arginine, histidine; (3) non polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, Substitution of single amino acids within these families does not have a major effect on the biological activity. The proteins can have one or more (e.g. 1, 2, 3, 4, 5, 6, 7, 8, 9, 10, etc.) single amino acid deletions relative to the disclosed protein sequences. The proteins may also include one or more (e.g. 1, 2, 3, 4, 5, 6, 7, 8, 9, 10, etc.) insertions (e.g. each of 1, 2, 3, 4 or 5 amino acids) relative to the disclosed protein sequences.

In some embodiments, a “nutritional serine protease inhibitor” can be a mature secreted form, or a substantially identical variant peptide thereof, functional homolog, or active peptide fragment thereof comprising a consecutive amino acid sequence of the nutritional serine protease inhibitor. A nutritional serine protease inhibitor can inhibit one or more of trypsin, chymotrypsin, and elastase enzymes. A nutritional serine protease inhibitor can be derived from an edible nutrient source, and is capable of exhibiting serine protease inhibitor activity. In one example, a serine protease inhibitor is a soybean product. In some embodiments, a nutritional serine protease inhibitor is a nutritional trypsin inhibitor. A nutritional trypsin inhibitor can be an enzyme active soy product. A nutritional trypsin inhibitor can be an ovomucoid capable of exhibiting trypsin inhibitor activity.

The term “fragment” is used herein to define any non-full-length string of amino acid residues that are directly derived from, synthesized to be identical with, or synthesized to have a sequence identity of at least 90%, at least 95%, at least 97%, or at least 99% compared to the amino acid sequence of interest. In some non-limiting embodiments, a fragment is capable of exhibiting trypsin inhibitor activity. The peptide fragment can comprise at the most 52 consecutive amino acid residues, at the most 50 consecutive amino acid residues, at the most 40 consecutive amino acid residues, at the most 30 consecutive amino acid residues, from 15 to 30 consecutive amino acid residues, or from 18 to 25 consecutive amino acid residues of the protein or peptide of interest.

A “functional homolog” can be defined as comprising the full-length amino acid sequence or fragment of the protein of interest with one or more, two or more, three or more, four or more, or five or more mutations in the amino acid residues. A functional homolog can retain at least 50%, 70%, 80%, 90%, 95%, 99% or more of the activity of the parent protein or peptide and can be, but is not limited to, a recombinant version of full length of fragmented protein with one or more, two or more, three or more, four or more, or five or more mutations.

Without being bound to any particular theory, the compositions illustrated by the embodiments provided for herein have been found to provide increased stability of colostrum with respect to digestion by various peptidases. These of colostrum compositions for treatment of ulcerative colitis or Crohn's disease, can be hampered by bioavailability of the colostrum compositions. Traditional approaches such as enema therapy or oral administration were unsuccessful because of various factors, including peptic digestion by pancreatic proteases. By combining colostrum with various nutritional serine protease inhibitors and stabilizers, resistance of colostrum to peptic digestion has been achieved. The amount of resistance was surprisingly synergistic, as certain compositions resulted in a trypsin inhibitor activity surpassing the theoretical inhibitor activity based on the individual components. This allowed for oral treatment of mice with colostrum containing compositions that resulted in protection in a mouse model of ulcerative colitis.

### Compositions

Embodiments disclosed herein are directed to a stabilized colostrum. In some embodiments, the composition comprises a pro-reparative factor; a nutritional serine protease inhibitor; and optionally a nutraceutically acceptable carrier or excipient. In some embodiments, a composition is provided comprising a pro-reparative factor, a nutritional trypsin inhibitor, and a nutraceutically acceptable carrier or excipient. In some embodiments, a composition is provided comprising a pro-reparative factor, a nutritional trypsin inhibitor, a stabilizing agent, and a nutraceutically acceptable carrier or excipient. In some embodiments, a composition is provided comprising a purified lactoferrin, a stabilizing agent, and optionally a nutraceutically acceptable carrier or excipient.

In certain embodiments, a composition further comprises a stabilizing agent. In some embodiments, the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, alginate plus acrylic polymer, or a combination thereof. In some embodiments, the stabilizing agent can be a matrix stabilizing agent a lipid stabilizing agent, a polysaccharide stabilizing agent, a disaccharide stabilizing agent, a sugar alcohol stabilizing agent, or a protein stabilizing agent. The stabilizing agent can be a microencapsulating agent, an acid resistant coating material, and/or enteric coating material.

In some embodiments, the pro-reparative factor is a colostrum, late colostrum, skim colostrum, partially defatted colostrum, a mixture of colostrum whey concentrate and whey protein concentrate, an egg product, an isolated pro-reparative peptide or protein, a nutraceutically acceptable salt thereof, or a combination thereof. In some embodiments, the colostrum is a bovine colostrum. In some embodiments, the bovine colostrum is skim bovine colostrum, partially defatted bovine colostrum, whole bovine colostrum, or a combination thereof.

In some embodiments, the pro-reparative factor can be a natural combination nutraceutical, an isolated pro-reparative peptide or protein, or a nutraceutically acceptable salt thereof. The natural combination nutraceutical can be a colostrum, an egg product, or a combination thereof.

In some embodiments, the colostrum can be a bovine colostrum. The bovine colostrum can be a skim bovine colostrum, partially defatted bovine colostrum, a whole bovine colostrum or a combination thereof.

In some embodiments, the isolated pro-reparative peptide or protein can be a recombinant or isolated pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), Vascular Endothelial Growth Factor (VEGF), Milk fat globule-epidermal growth factor 8 (MFG-E8), or nutraceutically acceptable salt thereof.

In some embodiments, a therapeutically effective amount of an isolated pro-reparative peptide or protein, or nutraceutically acceptable salt thereof, may be from about 0.001 mg/kg to about 10 mg/kg, about 0.005 mg/kg to about 5 mg/kg, about 0.01 mg/kg to about 1 mg/kg, or from about 20 μg/kg to about 200 μg/kg body weight of the subject per day.

In some embodiments, a therapeutically effective amount of an isolated pro-reparative peptide or protein, or nutraceutically acceptable salt thereof, or nutraceutically acceptable salt thereof, in compositions disclosed herein can be from about 0.01 mg to about 500 mg, about 0.05 mg to about 80 mg, about 0.1 to about 50 mg, about 0.5 to about 10 mg, or from about 1 mg to about 5 mg per day.

In some embodiments, a nutritional serine protease inhibitor is a nutritional trypsin inhibitor, wherein the nutritional trypsin inhibitor is an enzyme active soy product, an ovomucoid, a soybean trypsin inhibitor, or a combination thereof. In some embodiments, the enzyme active soy product is raw soy flour, raw soy meal, raw soy product, minimally processed soybean product, or a combination thereof. In some embodiments, an enzyme active soy product is an enzyme active soy flour. In some embodiments, a nutritional trypsin inhibitor can include enzyme active plant products from pigeon pea, chickpea, lentil, moth bean, green gram, lima bean, black gram, dry beans, pea, cowpea, tepary bean, or Faba/broad bean, so long as the trypsin inhibitor activity (TIA) of the nutritional trypsin inhibitor is at least about 10 trypsin units inhibited (TUI)/mg sample (e.g., at least about 10, 11, 12, 13, 14, 15, 20, 30, 40, or more TUI/mg); and/or iii) Protein Dispersibility Index (PDI) value greater than 40 (e.g, about greater than 40, 45, 50, 55, 60, 65, 70 or more DPI) when tested according to the American Oil Chemists Society (AOCS) Standard Procedure Ba 10b-09, which is hereby incorporated herein in its entirety. In some embodiments, the composition comprises about 40 wt % to about 90 wt % (e.g., about 40, 45, 50, 60, 70, 80, 90 wt % or more or any range between about 40 wt % and 90 wt %) colostrum; about 5 wt % to about 25 wt % enzyme active soy product (e.g., about 5, 10, 15, 20, 25 wt % or more or any range between about 5 wt % and 25 wt %); and about 5 wt % to about 40 wt % (e.g., about 5, 10, 15, 20, 25, 30, 35, 40 wt % or more or any range between about 5 wt % and 40 wt %) stabilizing agent. In certain specific embodiments, the composition may comprise about 40 wt % to about 95 wt % colostrum; about 0 wt % to about 25 wt % enzyme active soy product; and about 5 wt % to about 50 wt % of one or more combined stabilizing agents.

In some embodiments, the enzyme active soy product may exhibit: i) trypsin inhibitor activity (TIA) of at least 10 mg/g sample (e.g. about at least 10, 15, 20 or more mg/g) when tested according to the method of the International Organization for Standardization (ISO 14902:2001, which is hereby incorporated by reference herein in its entirety); ii) trypsin inhibitor activity (TIA) of at least 10 trypsin units inhibited (TUI)/mg sample (e.g., about at least 10, 11, 12, 13, 14, 15, 20, 25, 30 or more TUI/mg); and/or iii) Protein Dispersibility Index (PDI) value greater than 40 (e.g, about 40, 45, 50, 55 60, 65 or more) when tested according to the American Oil Chemists Society (AOCS) Standard Procedure Ba 10b-09, which is hereby incorporated herein in its entirety.

In some embodiments, the composition comprises a weight ratio of colostrum, late colostrum, skim colostrum, partially defatted colostrum, or mixture of colostrum whey concentrate and whey protein concentrate to enzyme active soy product from about 1:1 to about 20:1, and optionally from about 5:1 to about 15:1.

In certain embodiments, the composition comprises a pro-reparative factor; a nutritional serine protease inhibitor; and optionally a nutraceutically acceptable carrier or excipient. In some embodiments, the composition comprises about 40 wt % to about 95 wt % (e.g., about 40, 45, 50, 60, 70, 80, 90, 95 wt % or more or any range between about 40 wt % and 95 wt %) colostrum, late colostrum, skim colostrum, partially defatted colostrum, or mixture of colostrum whey concentrate and whey protein concentrate; and about 5 wt % to about 25 wt % (e.g., about 5, 10, 15, 20, 25 wt % or more or any range between about 5 wt % and 25 wt %) enzyme active soy product. In some embodiments, the composition comprises about 5 wt % to about 40 wt % (e.g., about 5, 10, 15, 20, 25, 30, 35, 40 wt % or more or any range between about 5 wt % and 40 wt %) stabilizing agent. In some embodiments, the composition further comprises soy lectin, medium-chain triglycerides, whey protein concentrate, non-fat dry milk, milk protein concentrate, or a combination thereof. In some embodiments, the composition comprises up to about 2 wt % (e.g., about 0.01, 0.1, 1.0, 1.5, 2.0 wt %) soy lecithin, up to about 2 wt % (e.g., about 0.01, 0.1, 1.0, 1.5, 2.0 wt %) medium-chain triglycerides, up to about 2 wt % (e.g., about 0.01, 0.1, 1.0, 1.5, 2.0 wt %) flavoring, up to about 50 wt % (e.g., about 5, 10, 20, 30, 40, 50 wt %) whey protein concentrate, up to about 10 wt % (e.g., about 1, 2, 5, 7, 10 wt %) non-fat dry milk, up to about 10 wt % (e.g., about 1, 2, 5, 7, 10 wt %) milk protein concentrate, or a combination thereof. In some embodiments, the ratio of colostrum whey concentrate to whey protein concentrate is from about 1:20 to about 20:1. In some embodiments, the ratio of colostrum whey concentrate to whey protein concentrate is about 60:40.

In some embodiments, the composition comprises a total protein content from about 50 wt % to about 80 wt % (e.g., about 50, 60, 70, 80 wt %) of the composition. In some embodiments, the composition comprises immunoglobulin G in an amount from about 15 wt % to about 60 wt % (e.g., about 15, 20, 30, 40, 50, 60 wt %) of the composition.

Embodiments disclosed herein are directed to a stabilized colostrum. In some embodiments, the composition comprises a pro-reparative factor, a stabilizing agent, and optionally a nutraceutically acceptable carrier or excipient.

In certain embodiments, the pro-reparative factor is a colostrum, late colostrum, skim colostrum, partially defatted colostrum, a mixture of colostrum whey concentrate and whey protein concentrate, an egg product, an isolated pro-reparative peptide or protein or a nutraceutically acceptable salt thereof, or combination thereof. In some embodiments, the colostrum is a bovine colostrum.

In some embodiments, the bovine colostrum is skim bovine colostrum, late bovine colostrum partially defatted bovine colostrum, a mixture of bovine colostrum whey concentrate and whey protein concentrate, whole bovine colostrum, or a combination thereof.

In some embodiments, the isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II) Platelet-derived growth factor (PDGF), Vascular Endothelial Growth Factor (VEGF), Milk fat globule-epidermal growth factor 8 (MFG-E8), or nutraceutically acceptable salts thereof.

In some embodiments, the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, and alginate plus acrylic polymer. In some embodiments, the stabilizing agent is calcium caseinate, sodium caseinate, and caseinate isolate from colostrum. In some embodiments, the composition comprises about 2 wt % to about 75 wt % (e.g., about 2, 5, 10, 20, 30, 40, 50, 60, 70, 75 wt %) calcium caseinate.

In some embodiments, the composition comprises about 40 wt % to about 98 wt % (e.g., about 40, 50, 60, 70, 80, 90, 98 wt %) colostrum, late colostrum, skim colostrum, partially defatted colostrum, or a mixture of colostrum whey concentrate and whey protein concentrate. In some embodiments, the composition further comprises about 25 wt % to about 40 wt % (e.g., about 25, 30, 35, 40 wt %) stabilizing agent. In some embodiments, the composition further comprises soy lectin, medium-chain triglycerides, whey protein concentrate, non-fat dry milk, milk protein concentrate, or a combination thereof. In some embodiments, the composition comprises up to about 2 wt % (e.g., about 0.01, 0.1, 1.0, 1.5, 2.0 wt %) soy lecithin, up to about 2 wt % (e.g., about 0.01, 0.1, 1.0, 1.5, 2.0 wt %) medium-chain triglycerides, up to about 2 wt % (e.g., about 0.01, 0.1, 1.0, 1.5, 2.0 wt %) flavoring, up to about 50 wt % (e.g., about 5, 10, 20, 30, 40, 50 wt %) whey protein concentrate, up to about 10 wt % non-fat dry milk (e.g., about 1, 2, 5, 7, 10 wt %), up to about 10 wt % (e.g., about 1, 2, 5, 7, 10 wt %), milk protein concentrate, or a combination thereof. In some embodiments, the ratio of colostrum whey concentrate to whey protein concentrate is from about 1:20 to about 20:1. In some embodiments, the ratio of colostrum whey concentrate to whey protein concentrate is about 60:40.

In some embodiments, the composition comprises a total protein content from about 50 wt % to about 80 wt % (e.g., about 50, 60, 70, 80 wt %), of the composition. In some embodiments, the composition comprises immunoglobulin G in an amount from about 15 wt % to about 60 wt % (e.g., about 15, 20, 30, 40, 50, 60 wt %) of the composition.

Embodiments disclosed herein are directed to a dosage form. In some embodiments, the dosage form comprises a composition comprising a pro-reparative factor; a nutritional protease inhibitor; and optionally a nutraceutically acceptable carrier or excipient. In some embodiments, the dosage form comprises a composition comprising a pro-reparative factor, a stabilizing agent, and optionally a nutraceutically acceptable carrier or excipient.

In certain embodiments, the dosage form is a powder, granule, tablet, orally disintegrating tablet, capsule, acid resistant capsule, troche, lozenge, nutrition bar, soft chew, hydrogel, gummy, yogurt, suspension, or ready to drink beverage. In some embodiments, the dosage form is an acid resistant capsule.

In some embodiments, a dietary supplement is provided comprising a composition according to any embodiments of the disclosure. The dietary supplement may be useful for supporting normal healthy physiology in a subject. The normal healthy physiology may be gastrointestinal physiology, immune physiology, musculoskeletal physiology, and respiratory physiology.

A nutritional serine protease inhibitor can comprise an amino acid sequence of any one of SEQ ID NOs: 1, 2, 3, 4, 5, 6, 7, 8, 9, 10, 11, 12, 13, 14, 15, 16, 17, 18, 19, 20, 21, 22, 23, 24 or 25, or an active peptide fragment, or a functional homolog thereof, which has the ability to exhibit trypsin inhibitor activity.

An active peptide fragment or functional homolog can be a protein or peptide that exhibits at least some sequence identity with the nutritional trypsin inhibitor of interest and has the ability to exhibit trypsin inhibitor activity. In a specific embodiment, the active protein or peptide fragment of the invention consists of at the most 150 amino acid residues, at the most 100 amino acid residues, at the most 50 amino acid residues, at the most 40 amino acid residues, at the most 30 amino acid residues, such as 15 to 150 consecutive amino acid residues, or 50 to 100 amino acid residues, of the trypsin inhibitor peptide of interest or a functional homolog thereof; the functional homolog having at the most three amino acid substitutions, such as two amino acid substitutions, or one amino acid substitution.

Bovine colostrum contains multiple pro-reparative factors, or growth factors, that influence cell growth, differentiation and function and cover a wide range of molecular weights including but not limited to Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), and the like. Over twenty different peptide growth factors have been described in BC. A pro-reparative factor can be BC itself, or an isolated or recombinant growth factor derived therefrom. Although active individually, their functions are interrelated as synergistic activity between various factors in colostrum have been demonstrated. For example, co-administration of bovine lactoferrin with EGF can result in synergistic activity in stimulating growth of the rat intestinal epithelial cell line IEC-18.

Insulin-like growth factors (IGFs, somatomedins) include IGF-I and IGF-II promote cell proliferation and differentiation and there is 100% sequence homology between the bovine and human IGFs. They are similar in structure to pro-insulin and IGFs can exert insulin-like effects at high concentrations. BC contains much higher concentrations of IGF-I than human colostrum (500 mg L⁻¹ compared to 18 mg L⁻¹) with lower concentrations in mature bovine milk (10 mg L⁻¹). These growth factors are relatively stable to both heat and acidic conditions and therefore survive the harsh conditions of both commercial milk processing and gastric acid to maintain biological activity. IGF-I can promote protein accretion, i.e. it is an anabolic agent and is at least partly responsible for mediating the growth-promoting activity of growth hormone. The high concentrations of IGFs in BC suggest they may be important in mediating local gut anabolic-reparative effects.

Epidermal growth factor receptor ligand family includes a group of polypeptides that have the common property of binding to the EGF receptor (also known as the c-erb1 receptor) and include EGF itself, TGF-α, mammary-derived growth factor II (MDGF-II), betacellulin and human milk growth factor III (HMGF-III). Other related polypeptides with these binding characteristics but not present in significant concentrations in colostrum are amphiregulin, and heparin binding EGF.

Epidermal growth factor (EGF) is a 53 amino acid peptide produced by the salivary glands and Brunner's glands of the duodenum. EGF is present in human colostrum (200 mg L⁻¹) and milk (30-50 mg L⁻¹) and in many other species. Milk-borne EGF is not deactivated under typical gastric proteolytic conditions, whereas adult gastric juice causes a major reduction in the bioactivity of EGF due to it being cleaved to an EGF1-49 form. Once EGF enters the small intestine, it is susceptible to proteolytic digestion under fasting conditions but preserved in the presence of ingested food proteins, including casein and trypsin inhibitors.

Transforming growth factor α (TGF-α) is a 50 amino acid molecule that is present in human colostrum and milk at much lower concentrations (2.2-7.2 mg′) than EGF. In contrast with EGF, TGF-α is produced within the mucosa throughout the gastrointestinal tract. Systemic administration of TGF-α stimulates gastrointestinal growth and repair, inhibits acid secretion, stimulates mucosal restitution after injury and increases gastric mucin. Within the small intestine and colon, TGF-α expression occurs mainly in the upper (non-proliferative) zones which suggests that its physiological role may be to influence differentiation and cell migration rather than cell proliferation.

Transforming growth factor β family of molecules is structurally distinct from TGF-α and, in most systems, inhibits proliferation, rather than stimulating it. There are at least 35 different isoforms of TGF-β and their major site of expression is in the superficial zones of the normal gastrointestinal tract. TGF-β has multiple functions and is a chemoattractant for neutrophils and stimulates epithelial cell migration at wound sites. It is therefore likely to be important in stimulating the early phase of the repair process where surviving cells from the wound edge migrate over the denuded area to re-establish epithelial continuity. TGF-β and TGF-β-like molecules are present in high concentrations in both bovine milk (1-2 mg^{L-1}) and colostrum (20-40 mg^{L-1}), these concentrations are sufficient to prevent indomethacin-induced gastric injury in rats.

Platelet-derived growth factor (PDGF) was originally identified from platelets but is also produced and secreted by macrophages. PDGF is acid-stable, consisting of 2 disulphide-linked polypeptides: chain A (14 kDa) and chain B (17 kDa). The dimer exists in 3 isoforms (AA, AB and BB) that bind to tyrosine kinase-type receptors. PDGF is a potent mitogen for fibroblasts and arterial smooth muscle cells. Oral administration of exogenous PDGF has been shown to enhance ulcer healing in animal models. Although PDGF is present in human and bovine milk and colostrum, most of the PDGF-like mitogenic activity in bovine milk is derived from bovine colostral growth factor, which shares sequence homology with PDGF.

Vascular endothelial growth factor (VEGF) is a homodimeric 34-42 kD heparin-binding glycoprotein with potent angiogenic, mitogenic and vascular permeability-enhancing factors that is related to PDGF. VEGF is present in human breast milk at about 75 mg^{L-1} during the first week of lactation and falls to about 25 mg^{L-1} during the second postnatal week. Due to its angiogenic activity, the VEGF content of BC may have value in enhancing local vascular supply in conditions such as peptic ulceration.

Milk fat globule-epidermal growth factor 8 (MFG-E8) is a secreted protein found in vertebrates. It was initially discovered as a critical component of the milk fat globule and is present in BC in high concentrations and may influence the immune and repair response of the suckling neonate. MFG-E8 contributes to the phagocytic removal of damaged and apoptotic cells from tissues, the induction of VEGF-mediated neovascularization, the maintenance of intestinal epithelial homeostasis, and the promotion of mucosal healing.

After ingestion, the pro-reparative growth factors in colostrum are inactivated and degraded by digestive enzymes including pepsin, trypsin, and chymotrypsin. Trypsin is a serine protease digestive enzyme that specifically hydrolyzes amides, peptides, and proteins at the C-terminus side of arginine and lysine residues. Trypsin inhibitors include soybean trypsin inhibitor and ovomucoid.

A nutritional protease inhibitor can be a nutritional trypsin inhibitor derived from a food product. A nutritional protease inhibitor can comprise a Kazal-type serine protease inhibitor domain or a Kunitz type serine protease inhibitor domain. A nutritional trypsin inhibitor can be a Soybean Trypsin Inhibitor (SBTI) having trypsin inhibitor activity, a soy product having trypsin inhibitor activity, or an ovomucoid having trypsin inhibitor activity. A nutritional protease inhibitor can be a casein. The casein can be obtained from a cow, sheep, goat, buffalo, or other mammal. The casein can be in the form of calcium caseinate, sodium caseinate or colloidal casein micelles.

A nutritional protease inhibitor can be any appropriate food product comprising trypsin inhibitor activity, or can be a protein or peptide concentrated, isolated, purified, purchased commercially, or prepared by any appropriate recombinant technique, or synthetic technique known in the art, or a substantially identical protein or peptide, comprising trypsin inhibitor activity.

A nutritional trypsin inhibitor can be any appropriate dietary trypsin inhibitor. Trypsin inhibitors are a type of serine protease inhibitor. Trypsin is an enzyme involved in the breakdown of proteins in the digestive process. Trypsin inhibitor activity (TIA) levels are particularly high in soybeans, but can also be found in found in various legumes, such as cowpeas, pinto beans, pigeon peas, kidney beans, moth beans and navy beans, as well as chickpeas and mung beans. Certain other foods may exhibit low levels of trypsin inhibitor activity. Trypsin inhibitor activity (TIA) can be destroyed by processing, particularly processing involving heat or other protein denaturing processes. Traditionally, trypsin inhibitors are considered to be anti-nutritional factors. Therefore, soybean products require heat processing to achieve maximum nutritional value. In contrast, the present disclosure provides a nutritional serine protease inhibitor that is capable of exhibiting at least a minimum level of trypsin inhibitor activity. For example, a nutritional serine protease inhibitor is useful to stabilize or prolong the activity of the pro-reparative factors and immune factors in colostrum in the gastrointestinal tract, for example, in the lower small intestine.

### Enzyme Active Soybean Products

The nutritional trypsin inhibitor can be an enzyme active soybean product comprising at least 10 mg/g sample trypsin inhibitor activity (TIA) (e.g., about at least 10, 15, 20, 30, 35, 40 mg/g TIA or more) in a trypsin inhibition assay. In some embodiments, a nutritional trypsin inhibitor can be an enzyme active soybean product comprising at least 20 mg/g sample trypsin inhibitor activity (TIA) (e.g., about at least 20, 30, 35, 40, 50, 60 mg/g TIA or more) in a trypsin inhibition assay.

Glycine (soybean, soya bean) is a genus in the bean family Fabaceae. Cultivated soybean is Glycine max. Two major types of trypsin inhibitors are found in soy: Kunitz trypsin inhibitor (KTI) and Bowman-Birk inhibitor (BBI). KTI is a large (˜20,100 daltons), strong inhibitor of trypsin and weakly binds chymotrypsin, while BBI is much smaller (˜8,000 daltons) and inhibits both trypsin and chymotrypsin. Many soy products contain both types of inhibitors including soy flours, although some soy foods include much lower amounts. Both inhibitors traditionally are considered to have anti-nutritive effects in the body, affecting digestion by hindering protein hydrolysis and activation of other enzymes in the gut. In soy, KTI is found in much larger concentrations than BBI. In addition, the survival in rat small intestine of soybean Kunitz or Bowman-Birk trypsin inhibitors was studied and although only a minor amount (4.8%) of the Bowman-Birk inhibitor survived, most of the Kunitz inhibitor (76%) remained immunochemically intact. Whole soybeans contain 17-27 mg/gram sample of trypsin inhibitor activity (TIA); raw soy flour 28-32 mg/g sample TIA; toasted soy flour 8-9 mg/g sample TIA; and isolate 1-30 mg/g sample TIA. Trypsin inhibitors can be inactivated by heat or chemical denaturization.

Soybean trypsin inhibitor (SBTI, Kunitz trypsin inhibitor, KTI) belongs to the serpins-serine protease inhibitor family and is one of several protease inhibitors found in soybeans. Trypsin is inhibited at a molar ratio of 1:1, while chymotrypsin and plasmin are inhibited to a lesser extent. SBTI will also inhibit other serine proteases. SBTI is commercially available, for example, from Roche, Calif., Gibco® Soybean Trypsin Inhibitor, ThermoFisher Scientific, or Sigma-Aldrich.

A soy product can be any suitable enzyme active soy product. Suitable soy products comprise at least 10 mg/g sample trypsin inhibitor activity (TIA) (e.g., about at least 10, 15, 20, 30, 35, 40 mg/g TIA or more).

In some embodiments, a nutritional trypsin inhibitor is a soybean trypsin inhibitor. In some embodiments, the soybean trypsin inhibitor comprises the amino acid sequence of any one of SEQ ID NOs: 1, 2, 3, 4, 20, 21, 22, 23, 24, or 25 or is a substantially identical, homolog, or active fragment thereof.

The soybean trypsin inhibitor (SBTI) can be a Kunitz soybean trypsin inhibitor. The soybean trypsin inhibitor can comprise an amino acid sequence having an accession number 1BA7_B or

| | |
|---|---|
| 1 | DFVLDNEGNP LENGGTYYIL SDITAFGGIR AAPTGNERCP LTVVQSRNEL DKGIGTIISS |
| 61 | PYRIRFIAEG HPLSLKFDSF AVIMLCVGIP TEWSVVEDLP EGPAVKIGEN KDAMDGWFRL |
| 121 | ERVSDDEFNN YKLVFCPQQA EDDKCGDIGI SIDHDDGTRR LVVSKNKPLV VQFQKLDKES |
| 181 | L |

(SEQ ID NO: 1) (181 amino acids), or a substantially identical variant, functional homolog, or active fragment thereof, comprising trypsin inhibitor activity.

The soybean trypsin inhibitor (SBTI) can comprise an amino acid sequence having an accession number 1BA7_A or

| | |
|---|---|
| 1 | DFVLDNEGNP LENGGTYYIL SDITAFGGIR AAPTGNERCP LTVVQSRNEL DKGIGTIISS |
| 61 | PYRIRFIAEG HPLSLKFDSF AVIMLCVGIP TEWSVVEDLP EGPAVKIGEN KDAMDGWFRL |
| 121 | ERVSDDEFNN YKLVFCPQQA EDDKCGDIGI SIDHDDGTRR LVVSKNKPLV VQFQKLDKES |
| 181 | L |

(SEQ ID NO: 2) (181 amino acids), or a substantially identical variant, functional homolog, or active fragment thereof, comprising trypsin inhibitor activity.

The soybean trypsin inhibitor (SBTI) can comprise an amino acid sequence having an accession number 1AVU_A or

| | |
|---|---|
| 1 | DFVLDNEGNP LENGGTYYIL SDITAFGGIR AAPTGNERCP LTVVQSRNEL DKGIGTIISS |
| 61 | PYRIRFIAEG HPLSLKFDSF AVIMLCVGIP TEWSVVEDLP EGPAVKIGEN KDAMDGWFRL |
| 121 | ERVSDDEFNN YKLVFCPQQA EDDKCGDIGI SIDHDDGTRR LVVSKNKPLV VQFQKLDKES |
| 181 | L |

(SEQ ID NO: 3) (181 amino acids), or a substantially identical variant, functional homolog, or active fragment thereof, comprising trypsin inhibitor activity.

The soybean trypsin inhibitor (SBTI) can comprise an amino acid sequence having an accession number TISYC or

| | |
|---|---|
| 1 | DFVLDNEGNP LENGGTYYIL SDITAFGGIR AAPTGNERCP LTVVQSRNEL DKGIETIISS |
| 61 | PYRIRFIAEG HPLSLKFDSF AVIMLCVGIP TEWSVVEDLP EGPAVKIGEN KDAMDGWFRL |
| 121 | ERVSDDEFNN YKLVFCPQQA EDDKCGDIGI SIDHDDGTRR LVVSKNKPLV VQFQKLDKES |
| 181 | L |

(SEQ ID NO: 4) (181 amino acids), or a substantially identical variant, functional homolog, or active fragment thereof, comprising trypsin inhibitor activity.

In some embodiments, a nutritional serine protease inhibitor can be a Bowman-Birk type proteinase inhibitor. A Bowman-Birk proteinase type inhibitor can be derived from Glycine max. In some embodiments, a Bowman-Birk proteinase type inhibitor can comprise an amino acid sequence having accession number AF106676.1 or

| | |
|---|---|
| 1 | DDESSKPCCD QCACTKSNPP QCRCSDMRLN SCHSACKSCI CA |

(SEQ ID NO: 20) (42 amino acids), or a substantially identical variant, functional homolog, or active fragment thereof, comprising trypsin inhibitor activity.

In some embodiments, the nutritional serine protease inhibitor can be a Bowman-Birk type proteinase inhibitor, partial, comprising an amino acid sequence having accession number AF106675.1 or

| | |
|---|---|
| 1 | KSCICALSYP AQCFCVDITD FCYEPCKPSE DDKEN |

(SEQ ID NO: 21) (35 amino acids), or a substantially identical variant, functional homolog, or active fragment thereof, comprising trypsin inhibitor activity.

In some embodiments, a nutritional serine protease inhibitor can be a Bowman-Birk type proteinase inhibitor, comprising an amino acid sequence having accession number P01055 or

| | |
|---|---|
| 1 | MVVLKVCLVL LFLVGGTTSA NLRLSKLGLL MKSDHQHSND DESSKPCCDQ CACTKSNPPQ |
| 61 | CRCSDMRLNS CHSACKSCIC ALSYPAQCFC VDITDFCYEP CKPSEDDKEN |

(SEQ ID NO: 22) (110 amino acids), or a substantially identical variant, functional homolog, or active fragment thereof, comprising trypsin inhibitor activity.

In some embodiments, a nutritional serine protease inhibitor can be a Bowman-Birk type proteinase inhibitor precursor [Glycine max]. A Bowman-Birk type proteinase inhibitor precursor can comprise an amino acid sequence having accession number NM 001250058.3 or

| | |
|---|---|
| 1 | MGLKNNMVVL KVCLVLLFLV GGTTSANLRL SKLGLLMKSD HHQHSNDDES SKPCCDQCAC |
| 61 | TKSNPPQCRC SDMRLNSCHS ACKSCICALS YPAQCFCVDI TDFCYEPCKP SEDDKENY |

(SEQ ID NO: 23) (118 amino acids), or a substantially identical variant, functional homolog, or active fragment thereof, comprising trypsin inhibitor activity.

In some embodiments, a soybean trypsin inhibitor (SBTI) can be a Kunitz trypsin inhibitor [Glycine max] having an amino acid sequence having an accession number AB070269.1 or

| | |
|---|---|
| 1 | MKSTIFFLFA LFCAFTTSYL PSAIADFVLD NEGNPLENGG TYYILSDITA FGGIRAAPTG |
| 61 | NERCPLTVVQ SRNELDKGIG TIISSPYRIR FIAEGHPLSL KFDSFAVIML CVGIPTEWSV |
| 121 | VEDLPEGPAV KIGENKDAMD GWFKLERVSD DEFNNYKLVF CPQQAEDDKC GDIGISIDHD |
| 181 | DGTRRLVVSK NKPLVVQFQK LDKESLAKKN HGLSRSE |

(SEQ ID NO: 24) (217 amino acids), or a substantially identical variant, functional homolog, or active fragment thereof, comprising trypsin inhibitor activity.

In some embodiments, a soybean trypsin inhibitor (SBTI) can be a Kunitz trypsin inhibitor [Glycine soja] having accession number AB308133.1 or amino acid sequence

| | |
|---|---|
| 1 | MKSTIFFALF LFCAFTTSYL PSAIADFVLD NEGNPLENGG TYYILSDITA FGGIRAAPTG |
| 61 | NERCPLTVVQ SRNELDKGIG TIISSPYRIR FIAEGHPLSL KFDSFAVIML CAGIPTEWSV |
| 121 | VEDLPEGPAV KIGENKDAMD GWFRLERVSD DEFNNYKLVF CPQQAEDDKC GDIGISIDHD |
| 181 | DGTRRLVVSK NKPLVVQFQK LDKESLAKKN HGLSRSE |

(SEQ ID NO: 25) (217 amino acids), or a substantially identical variant, functional homolog, or active fragment thereof, comprising trypsin inhibitor activity.

The composition can include nutritional serine proteinase inhibitor in the range of about 0 g/day to about 10 g/day, about 0.1 g/day to about 8 g/day, about 0.5 g/day to about 5 g/day, or about 1 g/day to about 5 g/day, or about 0 wt % to about 10 wt %, about 0.1 wt % to about 8 wt %, or about 1 wt % to about 5 wt %.

An enzyme active soybean product is derived from soybeans and comprises at least about 10 mg/g product trypsin inhibitor activity (TIA) (e.g., about at least 10, 15, 20, 30, 35, 40 mg/g TIA or more). Enzyme active soybean product may be a raw soy flour, raw soy meal, raw soy product, or minimally processed soybean product. A soybean product can be a soy flour which passes through #100 screen. A soybean product can be a soy meal, which passes through a #16 screen. In some embodiments, A soybean product can be a raw soybean product, or a minimally processed soybean product. In some embodiments, a soybean product contains a measurable minimum level of trypsin inhibitor activity. A soybean product can be a non-defatted, full fat, or defatted soybean product. A composition can include from about 0 wt % to about 30 wt %, about 5 wt % to about 30 wt %, about 0.5 wt % to about 25 wt %, about 2 wt % to about 20 wt %, or about 5 wt % to about 15 wt % enzyme active soy product.

Suitable soybean products can include soybean trypsin inhibitor (SBTI) at various levels depending on processing. A nutritional trypsin inhibitor such as SBTI can exhibit trypsin inhibitor activity (TIA), which can be determined by any appropriate TIA inhibitor assay.

To determine trypsin inhibitor activity (TIA) in a nutritional serine protease inhibitor, nutritional trypsin inhibitor, soy product, SBTI, or ovomucoid, a trypsin inhibitor assay can be performed, for example, according to the method of Liu, K., Soybean Trypsin Inhibitor Assay: Further Improvement of the Standard Method Approved and Reapproved by American Oil Chemists' Society and American Association of Cereal Chemists International, J Am Chem Soc (2019) 96:635-645. In some embodiments, a nutritional trypsin inhibitor has a trypsin inhibitor activity greater than about 10 TUI/mg, greater than about 20 TUI/mg, greater than about 30 TUI/mg, or greater than about 40 TUI/mg when assayed according to the method of present Example 3, or Liu 2019.

Alternatively, to determine trypsin inhibitor activity (TIA), a trypsin inhibitor assay can be performed according to the method of ISO 14902:2001. (2012) International Organization for Standardization, standard 14902:2001. Animal feeding stuffs—Determination of trypsin inhibitor activity of soya products. Approved October 2001; Reapproved August 2012. Geneva, Switzerland. This International Standard specifies a method for the determination of the trypsin inhibitor activity (TIA) of soya products. This trypsin inhibitor activity is indicative of the degree of toasting of these products. The detection limit of the method is about 0.5 mg/g. A native trypsin from bovine pancreas can be employed such as Merck 24579. Briefly, trypsin inhibitors are extracted from a sample at pH 9.5. The remaining trypsin activity is measured by adding benzoyl-L-arginine-p-nitroanilide (L-BAPA) as substrate. The quantity of released p-nitroaniline is measured spectrometrically. The trypsin inhibitor activity is expressed in milligrams trypsin inhibited per gram sample. The nutritional trypsin inhibitor, e.g., an enzyme active soybean product, as defined herein may exhibit at least about 10 mg/g sample, or at least about 20 mg/g, sample trypsin inhibitor activity (TIA).

The Protein Dispersibility Index (PDI) may be used to determine the dispersible protein, for example, in a soybean product under the conditions of the test. The PDI test may be performed according to the method of American Oil Chemists Society (AOCS) Standard Procedure Ba 10b-09. In some embodiments, the soy product has a PDI greater than about 40, greater than about 50, greater than about 60, or greater than about 70 when tested according to AOCS Standard Procedure Ba 10b-09.

### Ovomucoid

The nutritional trypsin inhibitor can be an ovomucoid. Ovomucoid is a glycosylated protein and serine protease inhibitor that comprises about 11% of hen's egg white proteins. Ovomucoid is also known as egg white trypsin inhibitor. Ovomucoid is about 28 kD in size and is capable of inhibiting trypsin and chymotrypsin. Ovomucoid or trypsin inhibitor is abundant in most avian egg whites. The hen's egg protein is composed of 186 amino acid residues and is highly glycosylated. It is highly immunogenic and is known to account for many egg allergies. Ovomucoid inhibits digestive enzymes, such as trypsin, chymotrypsin, and elastase.

The ovomucoid can be any appropriate ovomucoid comprising trypsin inhibitor activity. The ovomucoid can be an isolated ovomucoid or a recombinant ovomucoid. The recombinant ovomucoid can be provided, for example, from bacterial, fungal, algal bio-production. For example, an ovomucoid can be a chicken ovomucoid, turkey ovomucoid, guinea fowl ovomucoid, or duck ovomucoid. An ovomucoid comprises trypsin inhibitor activity. Ovomucoid can be derived from an avian egg white, as an isolated, partially purified, purified material, or may be purchased commercially. For example, chicken ovomucoid can be derived or purified from chicken egg white or purchased from a commercial vendor such as Cusabio® technology LLC, or Sigma-Aldrich. In some embodiments, the ovomucoid comprises the amino acid sequence of any one of SEQ ID NOs: 5, 6, 7, 8, 9, 10, 11, 12, 13, 14, 15, 16, 17, 18, or 19, or is a substantially identical, homolog, or active fragment thereof, and is capable of exhibiting trypsin inhibitor activity.

Chicken ovomucoid, (OMCHI) is also known as Allergen Gal d 1 and is a dominant allergen in chicken egg white. Gal d 1 is known to be a trypsin inhibitor having three domains; however, the trypsin inhibitory activity primarily resides in the second domain. Kato et al., Biochemistry, 1987 Jan. 13; 26(1):193-201. doi: 10.1021/bi00375a027. Hypersensitivity to Gal d 1 occurs because of its ability to efficiently bind to immunoglobulin E (IgE). One or more cysteine residues can be subjected to point mutation to decrease ability to bind to IgE. In some embodiments, an ovomucoid comprises an amino acid comprising a point mutation selected from C192A and a C210A.

A chicken ovomucoid can comprise the amino acid sequence of ovomucoid isoform 1 precursor [Gallus gallus] having accession NP_001295423.1 or

| | |
|---|---|
| 1 | MAMAGVFVLF SFVLCGFLPD AAFGAEVDCS RFPNATDKEG KDVLVCNKDL RPICGTDGVT |
| 61 | YTNDCLLCAY SIEFGTNISK EHDGECKETV PMNCSSYANT TSEDGKVMVL CNRAFNPVCG |
| 121 | TDGVTYDNEC LLCAHKVEQG ASVDKRHDGG CRKELAAVSV DCSEYPKPDC TAEDRPLCGS |
| 181 | DNKTYGNKCN FCNAVVESNG TLTLSHFGKC |

(SEQ ID NO: 5) (210 amino acids), or a substantially identical variant, functional homolog, or active fragment thereof, comprising trypsin inhibitor activity.

A chicken ovomucoid can comprise the amino acid sequence of ovomucoid isoform 2 precursor [Gallus gallus] having accession NP_001106132.1 or

| | |
|---|---|
| 1 | MAMAGVFVLF SFVLCGFLPD AAFGAEVDCS RFPNATDKEG KDVLVCNKDL RPICGTDGVT |
| 61 | YTNDCLLCAY SIEFGTNISK EHDGECKETV PMNCSSYANT TSEDGKVMVL CNRAFNPVCG |
| 121 | TDGVTYDNEC LLCAHKVEQG ASVDKRHDGG CRKELAAVDC SEYPKPDCTA EDRPLCGSDN |
| 181 | KTYGNKCNFC NAVVESNGTL TLSHFGKC |

(SEQ ID NO: 6) (208 amino acids) or a substantially identical variant, functional homolog, or active fragment thereof, comprising trypsin inhibitor activity.

A chicken ovomucoid can comprise the amino acid sequence of ovomucoid [Gallus gallus] having accession ACJ04729.1 or

| | |
|---|---|
| 1 | MAMAGVFVLF SFVLCGFLPD AVFGAEVDCS RFPNATDMEG KDVLVCNKDL RPICGTDGVT |
| 61 | YTNDCLLCAY SVEFGTNISK EHDGECKETV PMNCSSYANT TSEDGKVMVL CNRAFNPVCG |
| 121 | TDGVTYDNEC LLCAHKVEQG ASVDKRHDGG CRKELAAVSV DCSEYPKPDC TAEDRPLGGS |
| 181 | DNKTYGNKCN FCNAVVESNG TLTLSHFGKC |

(SEQ ID NO: 7) (210 amino acids), or a substantially identical variant, functional homolog, or active fragment thereof, comprising trypsin inhibitor activity.

A chicken ovomucoid can comprise the amino acid sequence of ovomucoid having accession 0807280A or

| | |
|---|---|
| 1 | AEVDCSRFPN ATDKEGKDVL VCNKDLRPIC GTDGVTYNNE CLLCAYSIEF GTNISKEHDG |
| 61 | ECKETVPMNC SSYANTTSED GKVMVLCNRA FNPVCGTDGV TYDNECLLCA HKVEQGASVD |
| 121 | KRHDGECRKE LAAVSVDCSE YPKPDCTAED RPLCGSDNKT YGNKCNFCNA VVESNGTLTL |
| 181 | SHFGKC |

(SEQ ID NO: 8) (186 amino acids) or a substantially identical variant, functional homolog, or active fragment thereof, comprising trypsin inhibitor activity.

A chicken ovomucoid can comprise the amino acid sequence of ovomucoid isoform X2 [Gallus gallus] having accession XP_015149250.1 or

| | |
|---|---|
| 1 | MAMAGVFVLF SFVLCGFLPD AAFGAEVDCS RFPNATDKEG KDVLVCNKDL RPICGTDGVT |
| 61 | YTNDCLLCAY SIEFGTNISK EHDGECKETV PMNCSSYANT TSEDGKVMVL CNRAFNPVCG |
| 121 | TDGVTYDNEC LLCAHKVEQG ASVDKRHDGG CRKELAAVDC SEYPKPDCTA EDRPLCGSDN |
| 181 | KTYGNKCNFC NAVVESNGTL TLSHFGKC |

(SEQ ID NO: 9) (208 amino acids), or a substantially identical variant, functional homolog, or active fragment thereof, comprising trypsin inhibitor activity.

A chicken ovomucoid can comprise the amino acid sequence of ovomucoid isoform X1 [Gallus gallus] having accession XP_015149249.1 or

| | |
|---|---|
| 1 | MAMAGVFVLF SFVLCGFLPD AAFGAEVDCS RFPNATDKEG KDVLVCNKDL RPICGTDGVT |
| 61 | YTNDCLLCAY SIEFGTNISK EHDGECKETV PMNCSSYANT TSEDGKVMVL CNRAFNPVCG |
| 121 | TDGVTYDNEC LLCAHKVEQG ASVDKRHDGG CRKELAAVSV DCSEYPKPDC TAEDRPLGGS |
| 181 | DNKTYGNKCN FCNAVVESNG TLTLSHFGKC |

(SEQ ID NO: 10) (210 amino acids), or a substantially identical variant, functional homolog, or active fragment thereof, comprising trypsin inhibitor activity.

In some embodiments, a nutritional protease inhibitor can be any appropriate turkey ovomucoid. A turkey ovomucoid can be derived from eggs of a wild turkey or a domestic turkey (Meleagris gallopavo) or an ocellated turkey (Meleagris ocellata).

A turkey ovomucoid can comprise the amino acid sequence of ovomucoid [Meleagris gallopavo] having accession XP_031411566.1 or

| | |
|---|---|
| 1 | MAMAGIFVLF SFALCGFLPD AAFGVEVDCS RFPNTTNEEG KDVLVCTEDL RPICGTDGVT |
| 61 | HSECLLCAYN IEYGTNISKE HDGECREAVP MDCSRYPNTT NEEGKVMILC NKALNPVCGT |
| 121 | DGVTYDNECV LCAHNLEQGT SVGKKHDGGC RKELAAVSVD CSEYPKPACT LEYRPLCGSD |
| 181 | NKTYGNKCNF CNAVVESNGT LTLSHFGKC |

(SEQ ID NO: 11) (209 amino acids) or a substantially identical variant, functional homolog, or active fragment thereof, comprising trypsin inhibitor activity.

A turkey ovomucoid can comprise the amino acid sequence of ovomucoid [Meleagris gallopavo] having accession P68390.1 or

| | |
|---|---|
| 1 | VEVDCSRFPN TTNEEGKDVL VCTEDLRPIC GTDGVTHSEC LLCAYNIEYG TNISKEHDGE |
| 61 | CREAVPMDCS RYPNTTSEEG KVMILCNKAL NPVCGTDGVT YDNECVLCAH NLEQGTSVGK |
| 121 | KHDGECRKEL AAVSVDCSEY PKPACTLEYR PLCGSDNKTY GNKCNFCNAV VESNGTLTLS |
| 181 | HFGKC |

(SEQ ID NO: 12) (185 amino acids) or a substantially identical variant, functional homolog, or active fragment thereof, comprising trypsin inhibitor activity.

An avian ovomucoid can be that of a helmeted guinea fowl comprising the amino acid sequence of ovomucoid [Numida meleagris] having accession XP_021266564.1 or

| | |
|---|---|
| 1 | MAMAGVFVLF SFALCGFLPD AAFGVEVDCS RFPNATNEEG KDVLVCTEDL RPICGTDGVT |
| 61 | YSNDCLLCAY NIEYGTNISK EHDGECREAV PVDCSRYPNM TSEEGKVLIL CNKAFNPVCG |
| 121 | TDGVTYDNEC LLCAHNVEQG TSVGKKHDGE CRKELAAVDC SEYPKPACTM EYRPLCGSDN |
| 181 | KTYDNKCNFC NAVVESNGTL TLSHFGKC |

(SEQ ID NO: 13) (208 amino acids) or a substantially identical variant, functional homolog, or active fragment thereof, comprising trypsin inhibitor activity.

An avian ovomucoid can be a duck ovomucoid. A duck ovomucoid may comprise the amino acid sequence of ovomucoid, partial [Anas platyrhynchos] having accession EOA95437.1 or

| | |
|---|---|
| 1 | QVDCSRFPNT TNEEGKEVLL CTKELSPVCG TDGVTYSNEC LLCAYNIEYG TNISKDHDGE |
| 61 | CKEAVPADCS MYPNMTNEEG KMTLLCNKMF SPVCGTDGVT YDNECMLCAH NVEQGTSVGK |
| 121 | KYDGKCKKEV ATVSVDCSGY PKPACTMEYM PLCGSDNKTY GNKCNFCNAV V |

(SEQ ID NO: 14) (171 amino acids) or a substantially identical variant, functional homolog, or active fragment thereof, comprising trypsin inhibitor activity.

A duck ovomucoid can comprise the amino acid sequence of ovomucoid [Anas platyrhynchos] having accession XP_005027968.1 or

| | |
|---|---|
| 1 | MRTAGVVVLL ALALCCCPDT VALKGYCSNY VVPRNFCTLE YIPHCGSDGV TYANKCLFCN |
| 61 | AFLRSRRTLR LLYLREC |

(SEQ ID NO: 15) (77 amino acids) or a substantially identical variant, functional homolog, or active fragment thereof, comprising trypsin inhibitor activity.

A duck ovomucoid can comprise the amino acid sequence of ovomucoid having accession P68150.1 or

| | |
|---|---|
| 1 | VATVDCSGYP KPACTMEYMP LCGSDNKTYG NKCNFCNAVV DSNGTLTLSH FGEC |

(SEQ ID NO: 16) (54 amino acids) or a substantially identical variant, functional homolog, or active fragment thereof, comprising trypsin inhibitor activity.

A duck ovomucoid can comprise the amino acid sequence of ovomucoid having accession P68156.1 or

| | |
|---|---|
| 1 | VATVDCSGYP KPACTMEYMP LCGSDNKTYG NKCNFCNAVV DSNGTLTLSH FGEC |

(SEQ ID NO: 17) (54 amino acids) or a substantially identical variant, functional homolog, or active fragment thereof, comprising trypsin inhibitor activity.

A duck ovomucoid can comprise the amino acid sequence of ovomucoid isoform X2 [Anas platyrhynchos] having accession XP_038042576.1 or

| | |
|---|---|
| 1 | MVLSVLPPFT QVTLAQGHGA LTCCPRHPET CPELERSTSW RGIKVDCSRF PNTTNEEGKE |
| 61 | VLLCTKELSP VCGTDGVTYS NECLLCAYNI EYGTNISKDH DGECKEAVPA DCSMYPNMTN |
| 121 | EEGKMTLLCN KMFSPVCGTD GVTYDNECML CAHNVEQGTS VGKKYDGKCK KEVATVDCSG |
| 181 | YPKPACTMEY MPLCGSDNKT YGNKCNFCNA VVDSNGTLTL SHFGEC |

(SEQ ID NO: 18) (226 amino acids) or a substantially identical variant, functional homolog, or active fragment thereof, comprising trypsin inhibitor activity.

A duck ovomucoid can comprise the amino acid sequence of ovomucoid isoform X1 [Anas platyrhynchos] having accession XP_038042575.1 or

| | |
|---|---|
| 1 | MISTSSGQLQ DNISCITSKL PSLVLPPFTQ VTLAQGHGAL TCCPRHPETC PELERSTSWR |
| 61 | GIKVDCSRFP NTTNEEGKEV LLCTKELSPV CGTDGVTYSN ECLLCAYNIE YGTNISKDHD |
| 121 | GECKEAVPAD CSMYPNMTNE EGKMTLLCNK MFSPVCGTDG VTYDNECMLC AHNVEQGTSV |
| 181 | GKKYDGKCKK EVATVDCSGY PKPACTMEYM PLCGSDNKTY GNKCNFCNAV VDSNGTLTLS |
| 241 | HFGEC |

(SEQ ID NO: 19) (245 amino acids) or a substantially identical variant, functional homolog, or active fragment thereof, comprising trypsin inhibitor activity.

A composition can comprise ovomucoid at the equivalent dose of about 0 g/day to about 10 g/day, about 0.1 g/day to about 8 g/day, about 0.5 g/day to about 5 g/day, or about 1 g/day to about 5 g/day (e.g., about 0, 0.1, 0.5, 1, 2, 3, 4, 5, 6, 7, 8, 9, 10, 11, 12, 13, 14, 15 g/day), or about 0 wt % to about 10 wt %, about 0.1 wt % to about 8 wt %, or about 1 wt % to about 5 wt % (e.g., about 0, 0.1, 0.5, 1, 2, 3, 4, 5, 6, 7, 8, 9, 10, 11, 12, 13, 14, 15 wt %) ovomucoid.

### Compositions

The biological activity of pro-reparative factors and immune factors found in bovine colostrum is sensitive to digestive enzymes, as shown herein. These factors can only be effective if they survive stomach digestion and reach the small intestine intact.

In some embodiments, a method is provided for enhancing the bioavailability of orally administered pro-reparative factors and/or immune factors such as found in bovine colostrum through pre-treatment of dry powder compositions, creating stabilized and effective delivery systems.

Commercially available spray dried bovine colostrum powder can be pre-treated by co-spraying, encapsulating, and/or agglomerating to create a protective coating around the colostrum. The protective coating can be used for delayed release, increasing bioavailability, acid resistance, enteric coating, physical barrier, or to enhance small intestine release of the pro-reparative factors such as colostrum. The coating can be in the form of liposomes, micelles, or micro encapsulating structures coating or surrounding the colostrum particles. The protective coating can be in the form of an enteric coating. Enteric coatings can provide a degree of protection from stomach acid and pepsin early in the digestive process, allowing the pro-reparative factors and other acid-sensitive components to survive stomach digestion intact at a significantly higher rate and thus enhancing the activity of these factors at their target sites in the small intestine.

Stabilized colostrum compositions are provided for enteral administration. Compositions are provided comprising colostrum, a nutritional trypsin inhibitor, and optionally a stabilizing agent.

Colostrum can be bovine colostrum. Bovine colostrum can be full fat colostrum (whole colostrum), partially defatted colostrum, or fully defatted colostrum (skim colostrum). The colostrum can be obtained within about 24 hours, within about 48 hours, or within a few days of parturition (e.g., about 2, 5, 10, 12, 24, 36, 48 or 72 hours after parturition). Colostrum can be concentrated or isolated. Colostrum can be spray dried, lyophilized, agglomerated, and/or instantized. Commercial colostrum can include whole colostrum, partially defatted colostrum, or skim colostrum. Commercial whole colostrum can comprise about 7 wt % to about 30 wt %, or about 17.5 wt % to about 28 wt % (e.g., about 7, 10, 15, 17.5, 20, 22, 25, 28 wt %) fat by AOAC 932.06 method. Commercial skim colostrum may comprise about 0 wt % to about 6 wt %, or about 2 wt % to about 6 wt % (e.g., about 0, 1, 2, 3, 4, 5, 6 wt %) fat by AOAC 932.06 method. Colostrum can be pasteurized, instantized, and/or agglomerated. Commercial colostrum can be processed at low pressures and temperatures to preserve bioactivity. Commercial colostrum can be spray dried. Commercial colostrum is available from, for example, APS BioGroup, Inc. Phoenix, Ariz. or LaBelle Associates, Inc., Bellingham, Wash. In some embodiments, colostrum can contain whole and/or skim colostrum, and one or more of milk protein concentrate, non-fat dry milk, sweet whey, whey protein concentrate, medium-chain triglycerides, lecithin, and silicon dioxide. In some embodiments, colostrum can contain from about 80 wt % to about 99 wt % whole or skim colostrum (e.g., about 80, 85, 90, 95, 99 wt %), about 0 wt % to about 20 wt % (e.g., about 0, 2, 5, 10, 15, 20 wt %) whey protein concentrate (WPC), about 0 wt % to about 20 wt % (e.g., about 0, 2, 5, 10, 15, 20 wt %) nonfat dry milk (NFDM), about 0 wt % to about 20 wt % (e.g., about 0, 2, 5, 10, 15, 20 wt %) sweet whey, about 0 wt % to about 1 wt % (e.g., about 0, 0.01, 0.1, 0.5, 1.0 wt %) flavor, about 0 wt % to about 1 wt % (e.g., about 0, 0.01, 0.1, 0.5, 1.0 wt %) lecithin, about 0 wt % to about 1 wt % (e.g., about 0, 0.01, 0.1, 0.5, 1.0 wt %) medium-chain triglycerides. In some embodiments, whole colostrum may include about 89.5 wt % whole instantized colostrum, about 10 wt % WPC, about 0.5 wt % flavor. In some embodiments, skim colostrum may include about 97.5% skim colostrum, about 1 wt % milk protein concentrate (MPC) 70, about 1 wt % NFDM, and about 0.5 wt % flavor. In some embodiments, a composition comprises about 30 wt % to about 95 wt % colostrum, about 40 wt % to about 90 wt % colostrum, or about 50 wt % to about 75 wt % colostrum.

In some embodiments, a composition comprises a total protein content from about 50 wt % to about 80 wt % (e.g. about 50, 60, 70, 80 wt %) of the composition. In some embodiments, the composition comprises a total protein content from about 60 wt % to about 70 wt % of the composition.

In some embodiments, the composition comprises immunoglobulin G in an amount from about 15 wt % to about 60 wt % (e.g. about 15, 20, 30, 40, 50, 60 wt %) of the composition. In some embodiments, the composition comprises immunoglobulin G in an amount from about 20 wt % to about 30 wt % of the composition.

A nutritional trypsin inhibitor can be an enzyme active soy product, an isolated or recombinant soybean trypsin inhibitor, or an isolated or recombinant ovomucoid. In some embodiments, a nutritional trypsin inhibitor is enzyme active raw soy flour. In some embodiments, an enzyme active soy flour is mixed with colostrum prior to co-spraying.

As defined herein, a nutritional trypsin inhibitor is capable of exhibiting at least about 10 mg/g sample trypsin inhibitor activity (TIA), or at least about 20 mg/g sample TIA, as determined by trypsin inhibition. Enzyme active soy products can include whole soybeans, raw soy flour, raw soy meal, certain soy protein concentrates, certain soy protein isolates. In some embodiments, the enzyme active soy product is not soy tofu, soy milk, soy miso, or soy infant formula, because these generally exhibit low levels of TIA of less than about 10 mg/g sample TIA.

Optionally, colostrum composition can be “spiked” with an isolated or recombinant growth factor, such as Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), Vascular Endothelial Growth Factor (VEGF), Milk fat globule-epidermal growth factor 8 (MFG-E8), and the like, or a substantially identical variant, functional homolog, or active fragment thereof.

Optionally, a composition can comprise an egg product. An egg product may be present at from about 0 wt % to about 70 wt %, about 5 wt % to about 50 wt %, or about 30 wt % to about 40 wt % of the composition (e.g. about 0, 10, 20, 30, 40, 50, 60, 70 wt %). The egg product can be pasteurized, spray dried whole egg, egg yolk, or egg white. In some embodiments, the composition includes about 40 wt % to about 95 wt % colostrum and about 5 wt % to about 25 wt % nutritional trypsin inhibitor. In some embodiments, the composition includes about 35 wt % to about 95 wt % (e.g. about 35, 40, 50, 60, 70, 80, 90, 95 wt %) colostrum, about 0 wt % to about 60 wt % (e.g. about 0, 10, 20, 30, 40, 50, 60 wt %) egg product, and about 5 wt % to about 25 wt % (e.g. about 5, 10, 15, 20, 25 wt %) nutritional trypsin inhibitor.

A stabilizing agent can serve to stabilize a composition with respect to gastrointestinal conditions and/or storage conditions. A stabilizing agent can be used as a matrix material that is mixed or blended with the colostrum, and/or may be used as a coating material to the matrix composition or the dosage form. Stabilizing agent materials can be selected from the list of excipients provided herein, or as follows, for example, phospholipids (e.g., Lecithin, ADM), lecithin, a medium-chain triglyceride (MCT), an alginic acid, alginate, colostrum fat, ethyl cellulose aqueous dispersion (Surelease®, Colorcon), maize starch, shellac (e.g., dewaxed aqueous based 25%, Marcoat®, Emerson Resources), sodium bicarbonate, pseudolatex, pea starch, hydroxypropyl methyl cellulose (HPMC), water soluble HPMC (e.g., Methocel® F4M or K15M, Dupont), HPMC acetate succinate, maltitol powder (e.g., SweetPearl® (e.g., Roquette), maltodextrin (e.g., Kleptose®, Roquette), disaccharide (e.g., trehalose, Bolise® Treha), casein or a caseinate salt such as calcium caseinate, sodium caseinate, potassium caseinate, magnesium caseinate, whey protein concentrate, milk protein concentrate, sweet whey, non-fat dry milk, xanthan gum (e.g., ADM), polydextrose (e.g., Tate and Lyle, DuPont), prolamine derived from corn, water-insoluble protein isolated from corn (e.g., zein, FloZein), alginate plus ethyl cellulose aqueous dispersion (e.g., Nutrateric®, Colorcon), stearine, glyceryl stearates, hydrogenated vegetable oil, alginate plus ethyl cellulose (e.g., Protect® EN, Sensient), alginate plus maize starch (e.g., Eudraguard Natural, Evonik), hydroxypropyl pea starch (e.g., LyCoat®, Roquette), partially hydrogenated soybean oil, hydrogenated soybean oil, hypromellose acetate succinate (e.g., AQOAT®, Shin-Etsu), aqueous cellulose acetate phthalate polymer (e.g., Aquacoat® CPD, Colorcon), alginate plus acrylic polymer (e.g., Eudraguard® Control, Evonik), or ethyl cellulose aqueous dispersion colloidal (e.g., Aquacoat® ECD, Colorcon). Stabilizing agents can be present in the composition at about 0 wt % to about 50 wt %, about 0.5 wt % to about 40 wt %, about 5 wt % to about 40 wt %, about 1 wt % to about 30 wt %, or about 5 wt % to about 25 wt % (e.g. about 0, 10, 20, 30, 40, 50 wt %) compared to the total weight of the composition.

A stabilizing agent can be a matrix stabilizing agent. In some embodiments, a matrix stabilizing agent can be a stabilizing lipid. In some embodiments, a stabilizing lipid can be a phospholipid, lecithin, medium-chain triglyceride, hydrogenated soybean oil, partially hydrogenated soybean oil, stearine, exogenous casein, caseinate, or exogenous colostrum fat. In some embodiments, a matrix stabilizing agent can be a polysaccharide stabilizing agent or disaccharide stabilizing agent. For example, a polysaccharide stabilizing agent can be a starch, alginate, alginic acid, polydextrose, guar gum, xanthan gum, maltodextrin, hydroxypropyl methylcellulose (HPMC; hypromellose), hydroxypropyl cellulose, methylcellulose, hydroxyethyl cellulose, hydroxyethyl methylcellulose, carrageenan, or pectin. A disaccharide stabilizing agent can be trehalose, maltose, sucrose, lactose, lactulose, or cellobiose. A stabilizing disaccharide can be a reducing disaccharide. The stabilizing disaccharide can be a non-reducing disaccharide. In one example, a stabilizing disaccharide can be trehalose. A stabilizing agent can be a sugar alcohol stabilizing agent. A sugar alcohol stabilizing agent can be maltitol, mannitol, xylitol, sorbitol, erythritol. A stabilizing agent can be a stabilizing protein. A stabilizing protein can be a casein, a nutritionally acceptable salt such as calcium caseinate, magnesium caseinate, sodium caseinate, potassium caseinate, whey protein concentrate, whey protein isolate, milk protein concentrate, sweet whey, or non-fat dry milk.

Compositions can include an acid suppressant. An acid suppressant can be a pH adjusting agent. A pH adjusting agent can be a gastric antacid. A gastric antacid can be, for example, sodium bicarbonate, sodium sesquicarbonate, potassium bicarbonate, magnesium hydroxide, magnesium carbonate, magnesium oxide, magnesium phosphate, calcium carbonate, dibasic calcium phosphate, tribasic calcium phosphate, aluminum hydroxide, aluminum phosphate, dihydroxyaluminum aminoacetate, dihydroxyaluminum sodium carbonate. A pH adjusting agent can be calcium hydroxide, potassium hydroxide, sodium hydroxide, sodium carbonate. In some embodiments, a stabilizing agent may be a matrix stabilizing agent. A matrix stabilizing agent can be trehalose, polydextrose, or maltodextrin. A stabilizing agent can be a microencapsulating agent, acid resistant coating material, and/or enteric coating material. In some embodiments, a microencapsulating agent or enteric coating material can be an alginate plus ethyl cellulose aqueous dispersion (e.g., Nutrateric®, Colorcon), alginate plus ethyl cellulose (e.g., Protect® EN, Sensient), alginate plus maize starch (e.g., Eudraguard Natural, Evonik), hydroxypropyl pea starch (e.g., LyCoat®, Roquette), hypromellose acetate succinate (e.g., AQOAT®, Shin-Etsu), aqueous cellulose acetate phthalate polymer (e.g., Aquacoat® CPD, Colorcon), alginate plus acrylic polymer (e.g., Eudraguard® Control, Evonik), or ethyl cellulose aqueous dispersion colloidal (e.g., Aquacoat® ECD, Colorcon). An acid suppressant may include a pH adjusting agent. The pH adjusting agent may be any appropriate GRAS pH adjusting agent, so long as it does not significantly interfere with the nutritional serine protease inhibitor activity. In some embodiments, the pH adjusting agent may be selected from sodium bicarbonate, sodium sesquicarbonate, or potassium bicarbonate. The pH adjusting agent may be sodium bicarbonate. In some embodiments, the pH adjusting agent may be added in the range of about 0.5 wt % to about 25 wt %, or about 1 wt % to about 10 wt % to the composition. For example, the pH adjusting agent such as sodium bicarbonate may neutralize some of the acids prevalent in the stomach and may act to protect the pro-reparative growth factors from degradation due to pepsin digestion.

### Matrix Stabilization

In some embodiments, stabilizing agents can be used for matrix stabilization for co-spray drying or otherwise mixing with the compositions of the disclosure. Matrix materials include, for example, trehalose, polydextrose, calcium caseinate, sodium caseinate, modified pea starch (e.g., LyCoat®, Roquette®), Xanthan gum, ethyl cellulose aqueous dispersion (e.g., Surelease®, Colorcon), alginate plus maize starch (e.g., Eudraguard Natural, Evonik), and hydroxypropyl methylcellulose (e.g., Methocel F4M or K15M).

A stabilizing agent can comprise a medium-chain triglyceride (MCT). Medium-chain triglycerides can be a triglyceride having two or three fatty acid esters, each having an aliphatic tail of medium-chain fatty acids having about 6 to about 12 carbon atoms. MCTs are found in palm kernel oil and coconut oil and can be separated by fractionation. The aliphatic tails can include straight chain fatty acids or branched chain fatty acids.

A stabilizing agent can comprise a phospholipid. A phospholipid can be an egg phospholipid or a lecithin. Egg phospholipids can be found as components of egg yolk including phosphatidylcholine (PC), phosphatidylethanolamine (PE) and lysophosphatidylcholine (LPC). Phospohlipids (“PLs”) and are crucial biological components in cell membranes. They are found in the diet primarily as glycerophopholipids and sphingolipids. Dietary glycerophospholipids are absorbed in the gastrointestinal tract at a high rate of efficiency of above about 90%. PL content of chicken eggs is about 28% of total lipids by weight, and an average chicken egg yolk contains about 1.3 g of PL according to the US Department of Agriculture (U.S. Department of Agriculture, A.R.S. USDA National Nutrient Database for Standard Reference, Release 26. Nutrient Data Laboratory Home Page. USDA; Washington, D.C., USA: 2013). PL from chicken egg is well absorbed.

A stabilizing agent can comprise alginic acid (or algin) which is a hydrophilic polysaccharide derived from brown marine macroalgae. Salts of algin with minerals such as calcium or sodium are called alginates. When exposed to water, alginate forms a viscous gum. In the food and pharmaceutical industries, these gumming properties of alginates are used to give food texture, to provide bulk thickening, as a gelling agent and as a carrier for delivering hydrogels containing pharmaceutical actives. The gelling qualities of alginates slow digestion and reduce exposure to acids and pepsin in the stomach.

A stabilizing agent can comprise colostrum fat. Colostrum fat can be a by-product of commercial bovine colostrum preparation. Colostrum fat contains elevated levels of omega-6 and omega-3 fatty acids (FA) and cholesterol compared to milkfat.

A stabilizing agent can include a casein or a caseinate salt, such as calcium caseinate. Milk proteins can be divided into two groups: soluble proteins (whey proteins), and insoluble proteins (caseins), with both components providing nutritional and bioactive properties. Casein is a major phosphoprotein that accounts for about three quarters of proteins in cow milk and cheese. Studies have demonstrated that the co-presence of casein may partially protects epidermal growth factor (EGF) from digestion in humans. Casein and caseinate can be isolated from colostrum. Without being bound by theory, casein or a salt thereof such as calcium caseinate, sodium caseinate, etc. can act as a competitive enzyme substrate in order to protect pro-reparative factors.

### Microencapsulation

In some embodiments, a composition is microencapsulated. Microencapsulation is the process of coating particles with an outer coating. Resulting microparticles can have an average diameter in the micrometer range between about 1 μm and about 1000 μm, for example, about 5 μm to about 800 μm, about 100 μm to about 750 μm, or about 150 μm to about 600 μm. Microencapsulation can take the form of a simple coating, or the formation of liposomal or micelle structures. A liposome is a minute sac of phospholipid molecules having at least one lipid bilayer. Structurally, liposomes have a lipid bilayer separating the internal aqueous contents from the build aqueous phase. Micelles are also minute sacs of phospholipid molecules, but with at least one lipid monolayer with fatty acid core and polar surface (or polar core with fatty acid surface in the case of a reverse micelle). Both liposomes and micelles have been used to target the delivery of drugs. In some embodiments, liposomes or micelles can be used to improve the bioavailability of pro-reparative factors in bovine colostrum, particularly in the small intestine.

The present disclosure provides a method for preparing bovine colostrum liposomes using chicken egg phospholipid (PL). An aqueous solution of bovine colostrum can be mixed with egg PL at a particular time and temperature. Liposomes can spontaneously form, and then the mixture is spray dried at low temperatures. In another embodiment of the process, an aqueous mixture of bovine colostrum and sodium or calcium alginate is mixed with egg PL. Then the mixture is spray dried at low temperatures. Optionally, the compositions can be agglomerated and/or instantized.

The resulting composition comprising bovine colostrum with nutritional trypsin inhibitor in egg PL liposome form enhances bioavailability of bovine colostrum as measured by growth factor bioactivity in vitro, or in a DSS colitis model in vivo through stomach digestion by protecting pro-reparative factors from stomach acid and gastrointestinal enzymatic digestion.

### Coating Materials

Composition described herein can comprise a coating. A coating can be a protective coating. In some embodiments, a protective coating can comprise an enteric coating.

Any suitable protective coating can be employed. In some embodiments, a coating comprises Generally Recognized as Safe (GRAS) components. In some embodiments, a coating can be an enteric coating. A coating can be an acid resistant coating. An enteric coating can be a commercially available enteric coating. A protective coating can enhance stability of a composition in shelf storage and/or enhance stability with respect to bioactivity of the composition. A coating concentration can be in range of from about 1 wt % to about 25 wt %, about 3 wt % to about 20 wt %, or about 5 wt % to about 15 wt % (e.g., about 1, 5, 10, 15, 20, 25, wt %) of the total weight of the composition.

A coating can include a film former, plasticizer, topcoat, anti-caking agent, colorants, glidant, and/or flavorants.

Suitable film formers for use in the subject compositions and methods include, e.g., pH-dependent polymers, water-insoluble polymers, and low-melting hydrophobic materials, copolymers thereof, and mixtures thereof. In some embodiments, the film former is a compound that is Generally Recognized as Safe (“GRAS”) by the U.S. Food and Drug. Film formers are described in, e.g., EP 2916828A1.

Examples of suitable water-insoluble polymers include, but are not limited to, ethyl cellulose, polyvinyl alcohols, polyvinyl acetate, polycaprolactones, cellulose acetate and its derivatives, acrylates, methacrylates, acrylic acid copolymers, copolymers thereof, and mixtures thereof. Suitable low-melting hydrophobic materials include, but are not limited to, fats, fatty acid esters, phospholipids, waxes, and mixtures thereof. Examples of suitable fats include, but are not limited to, hydrogenated vegetable oils such as for example cocoa butter, hydrogenated palm kernel oil, hydrogenated cottonseed oil, hydrogenated sunflower oil, and hydrogenated soybean oil, free fatty acids and their salts, and mixtures thereof. Examples of suitable fatty acid esters include, but are not limited to, sucrose fatty acid esters, mono-, di-, and tri-glycerides, glyceryl behenate, glyceryl palmitostearate, glyceryl monostearate, glyceryl tristearate, glyceryl trilaurylate, glyceryl myristate, GlycoWax-932, lauroyl macrogol-32 glycerides, stearoyl macrogol-32 glycerides, and mixtures thereof. Examples of suitable phospholipids include phosphotidyl choline, phosphotidyl serene, phosphotidyl enositol, phosphotidic acid, and mixtures thereof. Examples of suitable waxes include, but are not limited to, carnauba wax, spermaceti wax, beeswax, candelilla wax, shellac wax, microcrystalline wax, and paraffin wax; fat-containing mixtures such as chocolate, and mixtures thereof. In one preferred embodiment, the film former is ethyl cellulose. Suitable pH-dependent polymers for use as film formers include, but are not limited to, enteric cellulose derivatives such as hydroxypropyl methylcellulose phthalate, hydroxypropyl methylcellulose acetate succinate, cellulose acetate phthalate; natural resins such as shellac and zein; enteric acetate derivatives such as polyvinylacetate phthalate, cellulose acetate phthalate, acetaldehyde dimethylcellulose acetate; and enteric acrylate derivatives such as polymethacrylate-based polymers such as poly(methacrylic acid, methyl methacrylate) 1:2 (which is commercially under the tradename EUDRAGIT™), and poly(methacrylic acid, methyl methacrylate) 1:1 (which is commercially available under the tradename EUDRAGIT™), and mixtures thereof.

A composition or dosage form can include a commercially available nutritional aqueous enteric coating comprising an employing a pH sensitive polymer that allows for delayed release in the gastrointestinal tract. For example, an enteric coating can remain intact in an acidic environment such as in the stomach (e.g., pH 1.5-3.5), which can help protect the encapsulated material. After passing through the stomach, a coating then disintegrates in the small intestine (duodenum) which has a more alkaline environment (e.g., pH 6.5-7.6). Nutrateric® (Colorcon Inc.) is a commercially available nutritional aqueous enteric coating comprising an employing a pH sensitive polymer comprising aqueous ethylcellulose with sodium alginate that allows for delayed release in the gastrointestinal tract. Protect™ EN (Sensient Pharmaceutical) is an enteric coating comprising aqueous shellac and sodium alginate.

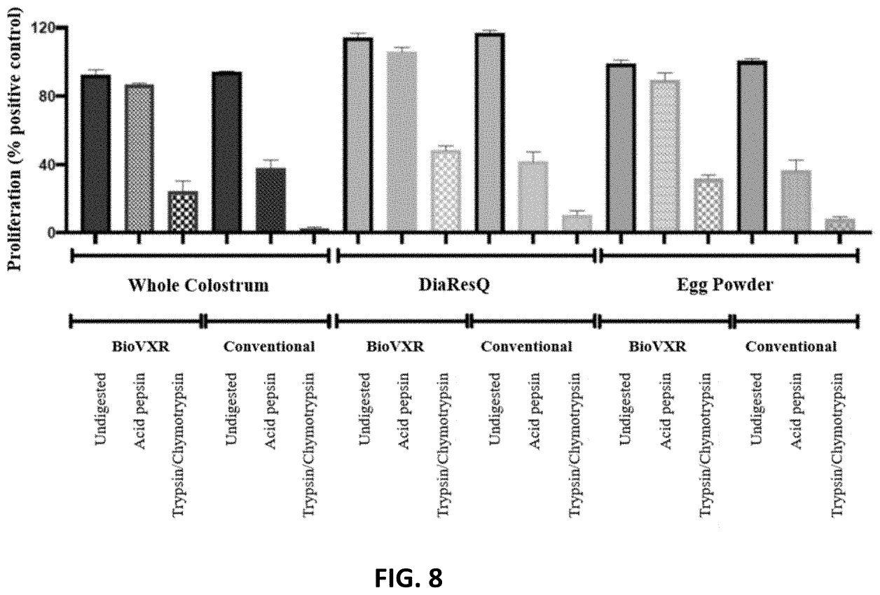

In some embodiments, a stabilizing agent may include an acid resistant coating or capsule shell. Suitable acid resistant coating or capsule shell compositions are described in, e.g., U.S. Pat. No. 9,452,141. An acid resistant coating or capsule shell can include a water-soluble enteric polymer. A water-soluble enteric polymer can have acid resistance, in other words, it can be insoluble under gastric condition (about pH 1.2) and can be soluble under intestinal condition (about pH 6.8). For example, a water-soluble enteric polymer can be pectin, propylene glycol alginate, or xanthan gum. An acid resistant coating or capsule shell can include a water-soluble film forming polymer. A water-soluble film forming polymer can be a pullulan, polyvinyl alcohol, modified starch, cellulose ester, or a gelatin. A cellulose ester can be a hydroxypropyl methylcellulose (HPMC), hydroxypropyl cellulose, methylcellulose, hydroxyethylcellulose, or hydroxyethyl methylcellulose. An acid resistant coating or capsule shell can include a gellan gum or carrageenan, and a salt such as sodium chloride, calcium chloride or potassium chloride as a gelling aid. As provided herein, acid resistant capsules can deliver more intact colostrum activity surviving the acid pepsin stage to the intestinal digestion stage (trypsin/chymotrypsin), as shown by improved proliferation (%) activity in AGS cells when compared to conventional non-acid resistant capsules, as shown in, e.g., FIG. 8.

In some embodiments, coating materials can include alginate plus ethyl cellulose aqueous dispersion (e.g., Nutrateric®, Colorcon), alginate plus maize starch (e.g., Eudraguard®, Evonik), hydrogenated soybean oil, alginate plus shellac (e.g., Protect EN®, Sensient), ethyl cellulose dispersion with pseudolatex (e.g., Surelease®, Colorcon), hydroxypropyl pea starch (e.g., LCoat®, Roquette), shellac aqueous based 25% solution (e.g., Marcoat®, Emerson Resources), and hydroxypropyl methylcellulose acetate succinate (e.g., AQOAT®, Shin-Etsu).

Optionally, plasticizers such as triethyl citrate or polyethylene glycol as well as anti-caking agents such are glyceryl monostearate or talc can also be included in enteric coating compositions.

In another embodiment, methods further comprise applying a topcoat, which can be a film former, to the enteric coating composition. In one embodiment, a topcoat can be a Generally Recognized as Safe (GRAS) film forming ingredient. In another embodiment, a topcoat can be selected from the group consisting of hydroxypropylmethyl-cellulose (HMPC), polyvinyl alcohol (PVA), or an amino methacrylate copolymer such as Eudragit E.

In some embodiments, representative stabilized colostrum compositions are provided in Table 1.

TABLE 1
Example Stabilized Colostrum Compositions

| Component | Composition 1 | Composition 2 | Composition 3 | Composition 4 |
|---|---|---|---|---|
| Colostrum | 40 wt %-95 wt % | 40 wt %-90 wt % | 50 wt %-90 wt % | 40 wt %-95 wt % |
| Nutritional | 5 wt %-30 wt % | 5 wt %-25 wt % | 10 wt %-20 wt % | 0 wt %-30 wt % |
| Serine Protease | | | | |
| Inhibitor | | | | |
| Stabilizing | 0 wt %-50 wt % | 5 wt %-40 wt % | 5 wt %-30 wt % | 5 wt %-50 wt % |
| agent(s) | | | | |

In some embodiments, the disclosure provides a stabilized composition comprising about 40 wt % to about 95 wt % of a colostrum, about 5 wt % to about 30 wt % of an enzyme active soy product, and about 0 wt % to about 50 wt % of a stabilizing agent. In some embodiments, the disclosure provides a stabilized composition comprising about 40 wt % to about 90 wt % of a colostrum, about 5 wt % to about 25 wt % of an enzyme active soy product, and about 5 wt % to about 40 wt % of a stabilizing agent. In some embodiments, the disclosure provides a stabilized composition comprising about 50 wt % to about 90 wt % of a colostrum, about 10 wt % to about 20 wt % of an enzyme active soy product, and about 5 wt % to about 30 wt % of a stabilizing agent. In some embodiments, the disclosure provides a stabilized composition comprising about 40 wt % to about 95 wt % of a colostrum, about 0 wt % to about 30 wt % of an enzyme active soy product, and about 5 wt % to about 50 wt % of a stabilizing agent.

The disclosure provides for a composition comprising a purified lactoferrin, a stabilizing agent, and optionally a nutraceutically acceptable carrier or excipient. The lactoferrin can be present at about 1.0, 1.5, 2.0, 2.5, 3.0, 3.5, 4.0, 4.5, 5.0, 6.0, 7.0, 8.0, 9.0, 10.0 ng/ml or more. The lactoferrin can be present at about 0.1, 0.5, 1.0, 1.5, 2.0, 2.5, 3.0, 3.5, 4.0, 4.5, 5.0, 6.0, 7.0, 8.0, 9.0, 10.0, 11.0, 12.0, 13.0, 14.0, 15.0 mg/ml. The lactoferrin can be present in an amount of up to about 98 wt % (e.g., about 2, 5, 10, 20, 30, 40, 50, 60, 70, 75, 80, 90, 98 wt %). The lactoferrin can be derived from cow, sheep, goat, buffalo, or other mammal. The lactoferrin can be recombinant lactoferrin based on human, cow, sheep, goat, buffalo, or other mammalian lactoferrin. The stabilizing agent and nutraceutically acceptable carrier or excipient can be any stabilizing agent as described herein (e.g. casein, caseinate salt, polydextrose, trehalose, exogenous colostrum fat, stearine, alginate, alginate plus ethyl cellulose aqueous dispersion, alginate plus ethyl cellulose, alginate plus maize starch, alginate plus acrylic polymer, a matrix stabilizing agent, a lipid stabilizing agent, a polysaccharide stabilizing agent, a disaccharide stabilizing agent, a sugar alcohol stabilizing agent, a protein stabilizing agent, a microencapsulating agent, an acid resistant coating material, an enteric coating material, or combinations thereof). A stabilizing agent can be and about 2 wt % to about 90 wt % (e.g., about 2, 5, 10, 15, 20, 25, 30, 35, 40, 50, 60, 70, 80, 90 wt % or more or any range between about 2 wt % and 90 wt %) of the composition. In some aspects, the stabilizing agent is calcium caseinate, sodium caseinate, or caseinate isolate from colostrum. In some embodiments, the composition comprises about 2 wt % to about 90 wt % (e.g., about 2, 5, 10, 20, 30, 40, 50, 60, 70, 75, 80, 90 wt %) calcium caseinate.

### Method of Use

Embodiments provide methods of treating, alleviating, or preventing a disease or disorder associated with inflammation of the gastrointestinal tract in a subject in need thereof, comprising administering a composition to the subject, the composition comprising a therapeutically effective amount of a pro-reparative factor, an immune factor, or a combination thereof, a nutritional serine protease inhibitor, and optionally a nutraceutically acceptable carrier or excipient.

Embodiments disclosed herein are directed to methods of treating, alleviating, or preventing a disease or disorder associated with inflammation of the gastrointestinal tract in a subject in need thereof, comprising administering a composition to the subject, the composition comprising a therapeutically effective amount of a pro-reparative factor, an immune factor, or a combination thereof, a stabilizing agent, and optionally a nutraceutically acceptable carrier or excipient.

In certain embodiments, methods comprise use of a composition further comprising a stabilizing agent. In some embodiments, a stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, and alginate plus acrylic polymer, or a combination thereof.

In some embodiments, methods use pro-reparative factors that are colostrum, late colostrum, skim colostrum, partially defatted colostrum, a mixture of colostrum whey concentrate and whey protein concentrate, an egg product, an isolated pro-reparative peptide or protein, or a nutraceutically acceptable salt thereof, or a combination thereof. In some embodiments, colostrum is bovine colostrum. In some embodiments, bovine colostrum is skim bovine colostrum, late bovine colostrum, partially defatted bovine colostrum, a mixture of bovine colostrum whey concentrate and whey protein concentrate, partially defatted bovine colostrum, whole bovine colostrum, or a combination thereof.

In some embodiments, methods are provided wherein an isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II) Platelet-derived growth factor (PDGF), Vascular Endothelial Growth Factor (VEGF), Milk fat globule-epidermal growth factor 8 (MFG-E8), or nutraceutically acceptable salts thereof.

In some embodiments, wherein the therapeutically effective amount of the isolated pro-reparative peptide or protein, or nutraceutically acceptable salt thereof, is administered at from about 0.001 mg/kg to about 10 mg/kg, about 0.005 mg/kg to about 5 mg/kg, about 0.01 mg/kg to about 1 mg/kg, or from about 20 μg/kg to about 200 μg/kg (e.g. about 0.001, 0.01, 0.02, 0.1, 1.0, 2.0, 5, 10, 20, 30, 40, 50, 60, 70, 80, 90, 100, 125, 150, 175, 200, 225, 250 mg/kg) body weight of the subject per day. In some embodiments, the therapeutically effective amount of the isolated pro-reparative peptide or protein, or nutraceutically acceptable salt thereof, is administered at from about 0.01 mg to about 500 mg, about 0.05 mg to about 80 mg, about 0.1 to about 50 mg, about 0.5 to about 10 mg, or from about 1 mg to about 5 mg per day (e.g. about 0.1, 1.0, 5, 10, 20, 30, 40, 50, 60, 70, 80, 90, 100, 125, 150, 175, 200, 225, 250, 300, 400, 500, 600, 700 mg per day).

In some embodiments, methods are provided wherein the immune factor is an immunoglobulin, cytokine, an anti-microbial peptide, or a combination thereof.

In some embodiments, methods are provided wherein the nutritional serine protease inhibitor is a nutritional trypsin inhibitor. In some embodiments, a nutritional trypsin inhibitor is an enzyme active soy product, an ovomucoid, a soybean trypsin inhibitor, or a combination thereof.

In some embodiments, methods are provided wherein the composition comprises about 40 wt % to about 95 wt % (e.g. about 40, 50, 60, 70, 80, 90, 95, 99 wt %) colostrum, late colostrum, skim colostrum, partially defatted colostrum, or a mixture of colostrum whey concentrate and whey protein concentrate; and about 5 wt % to about 25 wt % (e.g. about 5, 10, 15, 20 wt %) enzyme active soy product.

In some embodiments, methods are provided wherein the composition comprises a stabilizing agent. In some embodiments, the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, alginate plus acrylic polymer, or a combination thereof. In some embodiments, the stabilizing agent can be a matrix stabilizing agent selected from the group consisting of a lipid stabilizing agent, a polysaccharide stabilizing agent, a disaccharide stabilizing agent, a sugar alcohol stabilizing agent, or a protein stabilizing agent. The stabilizing agent can be a microencapsulating agent, an acid resistant coating material, and/or enteric coating material. In some embodiments, the composition comprises about 5 wt % to about 40 wt % (e.g. about 5, 10, 20, 30, or 40 wt %) stabilizing agent.

In some embodiments, methods are provided wherein the composition further comprises soy lectin, medium-chain triglycerides, whey protein concentrate, non-fat dry milk, milk protein concentrate, or a combination thereof. In some embodiments, the composition comprises up to about 2 wt % (e.g., about 0.01, 0.1, 0.5, 1.0, 1.5, 2.0 wt %) soy lecithin, up to about 2 wt % (e.g., about 0.01, 0.1, 0.5, 1.0, 1.5, 2.0 wt %) medium-chain triglycerides, up to about 2 wt % flavoring (e.g., about 0.01, 0.1, 0.5, 1.0, 1.5, 2.0 wt %), up to about 50 wt % (e.g., about 5, 10, 15, 20, 30, 40, 50 wt %) whey protein concentrate, up to about 10 wt % (e.g., about 1.0, 2.0, 5.0, 7.0, 10 wt %) non-fat dry milk, up to about 10 wt % (e.g., about 1.0, 2.0, 5.0, 7.0, 10 wt %) milk protein concentrate, or a combination thereof. In some embodiments, the ratio of colostrum whey concentrate to whey protein concentrate is from about 1:20 to about 20:1. In some embodiments, the ratio of colostrum whey concentrate to whey protein concentrate is 60:40.

In some embodiments, methods are provided wherein a composition comprises a total protein content from about 50 wt % to about 80 wt % (e.g., about 50, 60, 70, 80 wt %) of the composition. In some embodiments, the composition comprises immunoglobulin G in an amount from about 15 wt % to about 60 wt % (e.g., about 15, 20, 30, 40, 50, 60 wt %) of the composition.

In some embodiments, methods are provided wherein the disease or disorder is inflammatory bowel disease, non-steroidal antiinflammatory drug (NSAID) gastrointestinal disorder, chemotherapy-induced mucositis, radiation-induced mucositis, pseudomembranous colitis, gastritis, peptic ulcers, necrotizing entercolitis, irritable bowel syndrome, leaky gut, small intestinal bacterial overgrowth (SIBO), non-ulcer dyspepsia, or functional dyspepsia. In some embodiments, the inflammatory bowel disease is ulcerative colitis, indeterminate colitis, or Crohn's disease.

In some embodiments, methods are provided wherein the composition is effective to reduce myeloperoxidase (MPO) levels in the subject.

In some embodiments, methods are provided wherein the composition is effective to significantly preserve activity of the pro-reparative factor or immune factor when subjected to gastric juice or hydrochloride (HCl)/pepsin digestion and chymotrypsin/trypsin digestion, when compared to activity of pro-reparative factor and or immune factor alone under the same conditions.

In some embodiments, methods further comprise co-administering an effective amount of one or more additional active agents to the subject. In some embodiments, the subject is a non-neonate human subject.

Embodiments disclosed herein are directed to methods of enhancing a vaccination antibody titer in a subject, comprising a) orally administering a therapeutically effective amount of a composition to the subject, the composition comprising: i) a pro-reparative factor, an immune factor, or a combination thereof; ii) a nutritional serine protease inhibitor; iii) optionally a nutraceutically acceptable carrier or excipient; and iv) optionally a stabilizing agent; and b) administering a vaccine to the subject.

In certain embodiments, the vaccine is a COVID-19 vaccine, influenza vaccine, pneumococcal vaccine, tetanus vaccine, tetanus-diphtheria-pertussis vaccine, measles-mumps-rubella vaccine, human papillomavirus vaccine, hepatitis A vaccine, hepatitis B vaccine, meningococcal vaccine, Haemophilus influenzae type b vaccine, or zoster vaccine.

In some embodiments, the vaccine is a veterinary vaccine, such as a distemper vaccine, rabies vaccine, parvovirus vaccine, avian influenza vaccine, equine West Nile virus vaccine, equine influenza virus vaccine, feline leukemia virus vaccine, lymphocytic choriomeningitis virus vaccine, bovine viral diarrhea virus vaccine, feline immunodeficiency virus vaccine, porcine herpesvirus vaccine, foot-and-mouth disease virus vaccine, bovine herpesvirus-1 related disease vaccine, or Newcastle disease virus vaccine.

### Dosage Forms

Compositions can be an enteral composition. A composition can be an oral composition. In some embodiments, the compositions provided herein can be provided in a solid form such as a powder, agglomerated powder, instantized powder, lyophilized powder, granule, tablet, orally disintegrating tablet, capsule, troche, or lozenge. In some embodiments, the composition may be in a ready to use form such as a solid food form, for example, a nutrition bar, soft chew, hydrogel, or gummies, a semi-solid form such as yogurts, or in a liquid form, for example, a suspension, or a ready to drink beverage. In some embodiments, the composition can be in an ointment, suppository, or nutrient enema. In some embodiments, the dosage form can be a spray dried powder. In some embodiments, the dosage form can be a spray dried, instantized powder in a ready to mix form.

In some embodiments, a dosage form is an oral dosage form such as a powder, capsule, tablet, troche, liquid, or caplet. The powder can be utilized in a capsule fill, or sold in a single dose packet meant to mix with a food such as applesauce or yogurt, or can be an effervescent powder formulation sold in a single dose packet and meant for suspension in a liquid. In one aspect, a capsule, tablet, lozenge is intended for ingestion by swallowing. In another aspect, a tablet, capsule, or lozenge is orally disintegrable. In one aspect, a tablet, capsule, lozenge or troche is a slow-release composition. In another aspect, a tablet, lozenge, troche or capsule is an immediate release composition. In another aspect, an oral composition can be a prepackaged liquid drink, wherein the formulation is suspended in a flavored liquid. In some aspects, a composition is in the form of a tablet, a capsule, or a powder meant to mix with a food, such as applesauce or yogurt. Although the compositions of the disclosure are primarily oral forms, other modes of administration such as enteral forms, such as gastrostomy form, or anal suppositories are contemplated.

Tablet, capsule, and caplet forms of the disclosure can comprise, aside from those components specified above, other various additives, such as vehicle, binder, disintegrating agent, lubricant, thickener, surfactant, osmotic pressure regulator, electrolyte, sweetener, flavoring, perfume, pigment, pH regulator and others appropriately as required.

In another embodiment, compositions and dosage forms of the disclosure can optionally further comprise one or more flavoring agents. An optional flavoring agent can be added to increase patient acceptability and compliance with the recommended dosing schedule. Flavoring agents that can be used include those flavors known to the skilled artisan, such as natural and artificial flavors. These flavorings can be chosen from synthetic flavor oils and flavoring aromatics and/or oils, oleoresins and extracts derived from plants, leaves, flowers, fruits, and so forth, and combinations thereof. Non-limiting representative flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Other useful flavorings are artificial, natural and synthetic fruit flavors such as vanilla, and citrus oils including, without limitation, lemon, orange, lime, grapefruit, and fruit essences including apple, pear, peach, grape, strawberry, raspberry, cherry, plum, pineapple, apricot and so forth. These flavoring agents can be used in liquid or solid form and can be used individually or in admixture. Commonly used flavors include mints such as peppermint, menthol, artificial vanilla, cinnamon derivatives, and various fruit flavors, whether employed individually or in admixture. Other useful flavorings include aldehydes and esters such as cinnamyl acetate, cinnamaldehyde, citral diethylacetal, dihydrocarvyl acetate, eugenyl formate, p-methylamisol, and so forth can be used. In a specific aspect, the flavoring is spearmint oil. The flavor is optionally present from about 0.1 wt % to about 5 wt % (e.g., about 0.1, 0.5, 1, 2, 3, 4, 5 wt %) by weight of the antiviral composition.

Tablets can be molded tablets or compressed tablets. Tablets can be formed by wet granulation, dry granulation, and direct compression. These techniques are known to one of skilled in the art and are described, for example, in the United States Pharmacopeia National Formulary USP XXII, 1990, pp. 1696-1697. Various vitamins can be added to composition. Tablets can optionally further comprise flavorings or sweeteners. In one aspect, a sweetened, flavored tablet is utilized as a lozenge to be dissolved in the mouth. The compositions of the disclosure can also be prepared in a chewable form or an effervescent form. For effervescent preparations, the manufacturing method in the disclosure is basically same as in the manufacturing method of the usual effervescent preparations such as effervescent tablets. That is, components are weighed, mixed, and prepared directly by the powder compression method, dry or wet granular compression method, etc. Orally disintegrable tablets are described, for example, in U.S. Pat. No. 7,431,942. Lozenges with a hard candy base can be prepared, for example, by the techniques of U.S. Pat. No. 6,316,008.

A liquid composition can further comprise other nutrients. Such liquid compositions can be prepared as described in, for example, U.S. Pat. No. 6,037,375. A nutrient liquid composition of the disclosure can include a colostrum and an enzyme active soy product, and is prepared in the same manner as the ordinary food and beverage, and other food materials can be appropriately added. Sweeteners such as organic acids and carbohydrates can also be added. Organic acid components include, for example, citric acid, tartaric acid, malic acid, and succinic acid. These organic acids are added usually in a range of about 100 mg/100 mL to about 1500 mg/100 mL, preferably about 250 mg/100 mL to about 800 mg/100 mL (e.g., about 100, 200, 250, 300, 400, 500, 600, 700, 800, 900 mg/100 mL), and the composition of the material in beverage form can be prepared.

Various sweeteners can be optionally used in the tablet, liquid, capsule, lozenge or troche formulations of the disclosure. Examples of carbohydrates and sweeteners include monosaccharides such as glucose and fructose, disaccharides such as maltose, sucrose, other ordinary sugars, sugar alcohols such as xylitol, sorbitol, glycerin and erythritol, polysaccharides such as dextrin and cyclodextrin, and oligosaccharides such as fructo-oligosaccharide, galacto-oligosaccharide and lacto-sucrose. Other sweeteners include natural sweeteners such as thaumatin, stevia extract, rebaudioside A, glycyrrhizinic acid, etc. and synthetic sweeteners such as saccharin, aspartame, etc. These carbohydrates can be also added as carbohydrate mixture such as isomerized sugar and refined sugar. The sweetener can be optionally present from about 0.1% to about 5% (e.g., about 0.1, 0.5, 1, 2, 3, 4, 5) by weight of the solid composition. The blending of the carbohydrates can be about 1 g in 100 mL to about 15 g in 100 mL of the beverage composition of the disclosure or about 3 g to about 12 g (e.g., about 1, 2, 3, 5, 7, 9, 10, 12, 15 g in 100 mL). The content of the oligosaccharide can be about 0.5 g to about 10 g or about 1 g to about 3 g (e.g., about about 0.5, 1, 2, 3, 4, 5, 6, 7, 8, 9, 10 g).

A liquid nutrient composition of the disclosure can be prepared by blending these components, and the method of preparation is not particularly limited, and all components may be blended simultaneously. In an embodiment, fat-soluble components can be preliminarily dissolved in oil, and water-soluble components in water, then the solution can be emulsified by using an emulsifier, so that the composition of the disclosure can be prepared.

Emulsification can be executed by using a proper emulsifying machine, for example, homo-mixer or high-pressure homogenizer, either by complete passing system or by circulation system. The emulsion after emulsification can be filtered by conventional process, and poured into proper containers and sterilized, so that a desired beverage product is obtained. Sterilization can be affected by pasteurization.

A liquid composition of the disclosure can be also prepared in an effervescent form. The effervescent form should contain, aside from the essential components of the disclosure, proper amounts of sodium carbonate and/or sodium hydrogen carbonate and neutralizing agent as foaming components. The neutralizing agent used herein can be an acidic compound capable of generating carbon dioxide by neutralizing sodium carbonate or sodium hydrogen carbonate. Such compounds include, for example, L-tartaric acid, citric acid, fumaric acid, ascorbic acid and other organic acid. Ascorbic acid possesses both the action of neutralizing agent and the action of antioxidant.

Compositions and dosage forms of the disclosure can be subjected to pasteurizing processing that involves high heat (UHT (ultra-high temperature; e.g. about 135° C. to about 140° C. for 2 to 4 sec), HTST (high temperature, short time; aka flash pasteurization, e.g., about 72° C. to about 74° C. for 15 to 20 sec), LTLT (low temperature, long time; e.g., about 63° C. for 30 min), and/or high-pressure processing (HPP), and/or other food or supplement related processes with high heat (gummies, yogurts, RTD beverages, baked nutrition bars, etc.).

### Improving Vaccination Antibody Titers

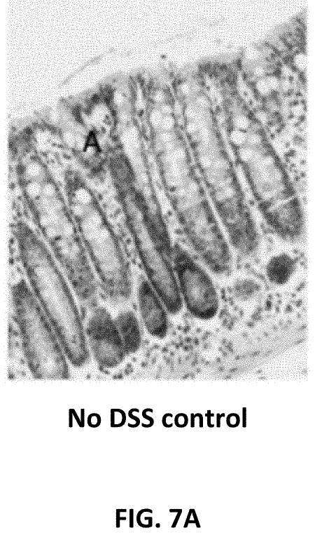

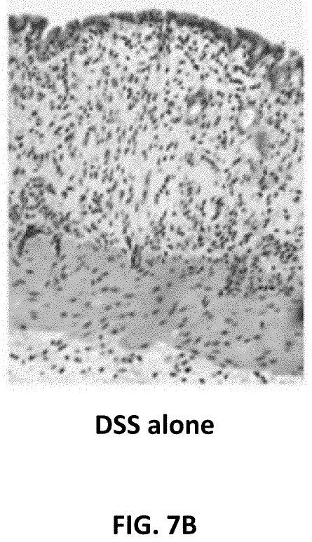

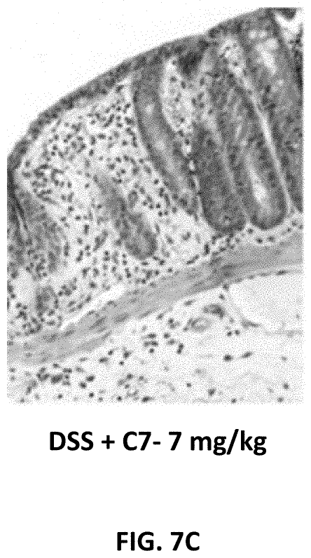

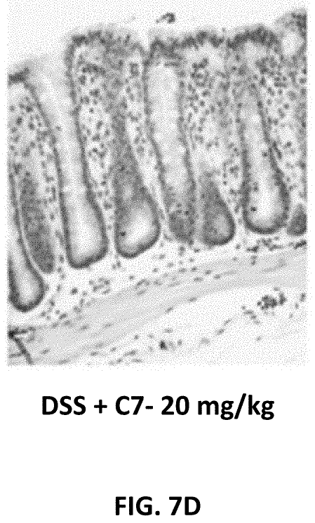

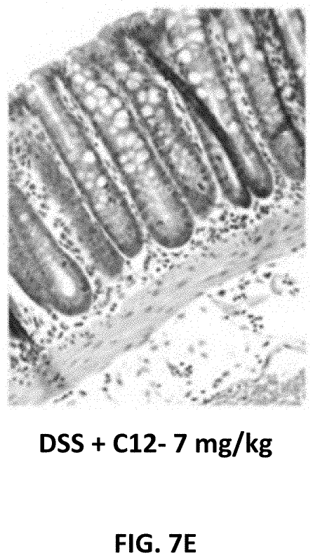

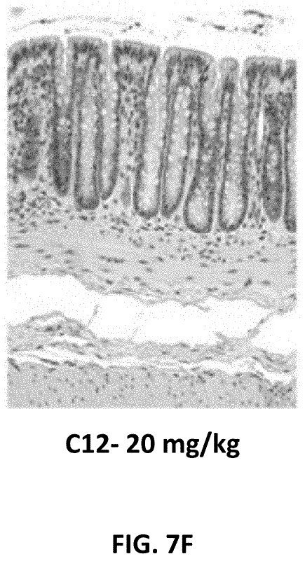

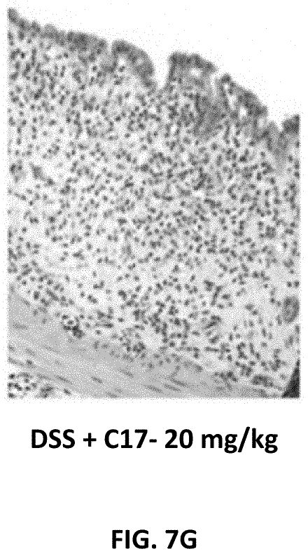

In some embodiments, the disclosure provides methods of enhancing a vaccination antibody titer in a subject receiving a vaccine. For example, a method of enhancing a vaccination antibody titer in a subject is provided comprising orally administering to the subject a therapeutically effective amount of a composition comprising: a pro-reparative factor and/or an immune factor; a nutritional serine protease inhibitor; a nutraceutically acceptable carrier or excipient; and optionally a stabilizing agent; and administering a vaccine to the subject. As shown in present Example 1, in the presence of pancreatic enzymes, nutritional protease inhibitors stabilize the growth factor activity of pro-reparative factors in BC and/or egg. In addition, evidence for improved immune factor stability is provided in Example 4, disclosing prototype colostrum compositions and the “numerical heat map” tables (FIG. 7A and FIG. 7B), showing improved immune bio-stability as IgG (μm/ml) and lactoferrin (ng/ml) when using certain stabilizing agents, or about 10 wt % nutritional serine protease inhibitor enzyme active soy flour, as described herein. Without being bound by theory, because orally administered pro-reparative factors and immune factors are stabilized when formulated in compositions of the disclosure, these compositions can be utilized for enhancing a vaccination antibody titer in a subject receiving a vaccine.

In some embodiments, the present disclosure provides methods of enhancing a vaccination antibody titer in a subject, comprising: orally administering a therapeutically effective amount of a composition to the subject, the composition comprising: i) a pro-reparative factor and/or an immune factor; ii) a nutritional serine protease inhibitor; iii) a nutraceutically acceptable carrier or excipient; and iv) optionally a stabilizing agent; and b) administering a vaccine to the subject. The vaccine can be selected from the group consisting of a COVID-19 vaccine, influenza vaccine, pneumococcal vaccine, tetanus vaccine, tetanus-diphtheria-pertussis vaccine, measles-mumps-rubella vaccine, human papillomavirus vaccine, hepatitis A vaccine, hepatitis B vaccine, meningococcal vaccine, Haemophilus influenzae type b vaccine, and a zoster vaccine. In some embodiments, the vaccine is a veterinary vaccine. The veterinary vaccine can be, for example, a distemper vaccine, rabies vaccine, parvovirus vaccine, avian influenza vaccine, equine West Nile virus vaccine, equine influenza virus vaccine, feline leukemia virus vaccine, lymphocytic choriomeningitis virus vaccine, bovine viral diarrhea virus vaccine, feline immunodeficiency virus vaccine, porcine herpesvirus vaccine, foot-and-mouth disease virus vaccine, bovine herpesvirus-1 related disease vaccine, or Newcastle disease virus vaccine.

### Stability

The compositions and dosage forms of the disclosure can be subjected to stability testing in real time (25° C. and 60%) and accelerated stability testing (40° C. and 75% RH) across different timeframes (1 month, 2 months, 3 months, 6 months, 12 months, 18 months, etc.).

### Administration

The compositions of the disclosure can be administered by an enteral route of administration. Methods of administration may include oral, buccal, sublingual, gastric, and rectal.

### Doses

The composition of the disclosure can be effective for treating, alleviating, decreasing severity of symptoms, preventing the onset, or reducing the incidence of a gastrointestinal disorder when taken on an intermittent or a regular basis over time. This could be a daily, in one or divided doses or taken on an intermittent basis. For example, a human age 15 or above averaging about 70 kg in weight who is not suffering yet from gastrointestinal disorder may take single or multiple doses on a powder basis of from about 0.3 g/day to about 100 g/day, about 0.5 g/day to about 33 g/day, about 1 g/day to about 15 g/day, or about 2 g/day to about 10 g/day (e.g., about 0.1, 0.5, 1, 5, 10, 15, 20, 30, 33 or more g/day).

The composition of the disclosure can be effective for dietary supplementation, in functional foods and beverages, and for supporting normal gastrointestinal and immune function when taken on an intermittent or a regular basis over time. This could be a daily, in one or divided doses or taken on an intermittent basis. For example, a human age 15 or above averaging about 70 kg in weight who is not suffering yet from gastrointestinal disorder may take single or multiple doses on a powder basis of from about 0.3 g/day to about 100 g/day, about 0.5 g/day to about 33 g/day, about 1 g/day to about 15 g/day, or about 2 g/day to about 10 g/day (e.g., about 0.1, 0.5, 1, 5, 10, 15, 20, 30, 33 or more g/day).

### Additional Embodiments

Embodiment A is directed to a composition comprising a pro-reparative factor, a nutritional protease inhibitor, and optionally a nutraceutically acceptable carrier or excipient.

In an embodiment B, the composition of embodiment A, wherein the composition further comprises a stabilizing agent.

In an embodiment C, the composition of embodiment B, wherein the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, alginate plus acrylic polymer, or a combination thereof.

In an embodiment D, the composition of any one of embodiments A to C, wherein the pro-reparative factor is a colostrum, late colostrum, skim colostrum, partially defatted colostrum, a mixture of colostrum whey concentrate and whey protein concentrate, an egg product, an isolated pro-reparative peptide or protein, a nutraceutically acceptable salt thereof, or a combination thereof.

In an embodiment E, the composition of embodiment D, wherein the colostrum is a bovine colostrum.

In an embodiment F, the composition of embodiment E, wherein the bovine colostrum is selected from skim bovine colostrum, partially defatted bovine colostrum, or whole bovine colostrum.

In an embodiment G, the composition of embodiment D, wherein the isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II) Platelet-derived growth factor (PDGF), Vascular Endothelial Growth Factor (VEGF), Milk fat globule-epidermal growth factor 8 (MFG-E8), or nutraceutically acceptable salts thereof.

In an embodiment H, the composition of any one of embodiments A to D, wherein the nutritional protease inhibitor is casein.

In an embodiment I, the composition of any one of embodiments A to D, wherein the nutritional protease inhibitor is a nutritional trypsin inhibitor.

In an embodiment J, the composition of embodiment H, wherein the nutritional trypsin inhibitor is an enzyme active soy product, an ovomucoid, a soybean trypsin inhibitor, or a combination thereof.

In an embodiment K, the composition of embodiment I, wherein the enzyme active soy product is raw soy flour, raw soy meal, raw soy product, minimally processed soybean product, or a combination thereof.

In an embodiment L, the composition of embodiment J, wherein the enzyme active soy product is an enzyme active soy flour.

In an embodiment M, the composition of embodiment K, comprising a weight ratio of colostrum, late colostrum, skim colostrum, partially defatted colostrum, or mixture of colostrum whey concentrate and whey protein concentrate to enzyme active soy product from about 1:1 to about 20:1, optionally from about 5:1 to about 15:1.

In an embodiment N, the composition of any one of embodiments A to K comprising: about 40 wt % to about 95 wt % colostrum, late colostrum, skim colostrum, partially defatted colostrum, or mixture of colostrum whey concentrate and whey protein concentrate; and about 5 wt % to about 25 wt % enzyme active soy product.

In an embodiment O, the composition of embodiment M, comprising about 5 wt % to about 40 wt % stabilizing agent.

In an embodiment P, the composition of embodiment N, comprising about 5 wt % to about 40 wt % nutritional protease inhibitor.

In an embodiment Q, the composition of embodiment N, comprising about 2 wt % to about 7 wt % casein.

In an embodiment R, the composition of embodiment N, further comprising soy lectin, medium-chain triglycerides, whey protein concentrate, non-fat dry milk, milk protein concentrate, or combinations thereof.

In an embodiment S, the composition of embodiment R, comprising up to about 2 wt % soy lecithin, up to about 2 wt % medium-chain triglycerides, up to about 2 wt % flavoring, up to about 50 wt % whey protein concentrate, up to about 10 wt % non-fat dry milk, up to about 10 wt % milk protein concentrate, or combinations thereof.

In an embodiment T, the composition of embodiment N, wherein the ratio of colostrum whey concentrate to whey protein concentrate is from about 1:20 to about 20:1.

In an embodiment U, the composition of embodiment N, wherein the ratio of colostrum whey concentrate to whey protein concentrate is 60:40.

In an embodiment V, the composition of any one of embodiments N to U, wherein the composition comprises a total protein content from about 50 wt % to about 80 wt % of the composition.

In an embodiment W, the composition of any one of embodiments N to U, wherein the composition comprises immunoglobulin Gin an amount from about 15 wt % to about 60 wt % of the composition.

Embodiment X is directed to a composition comprising a pro-reparative factor, a stabilizing agent, and optionally a nutraceutically acceptable carrier or excipient.

In an embodiment Y, the composition of embodiment X, wherein the pro-reparative factor is selected from a colostrum, late colostrum, skim colostrum, partially defatted colostrum, a-mixture of colostrum whey concentrate and whey protein concentrate, an egg product, an isolated pro-reparative peptide or protein or a nutraceutically acceptable salt thereof, or a combination thereof.

In an embodiment Z, the composition of embodiment Y, wherein the colostrum is a bovine colostrum.

In an embodiment AA, the composition of embodiment Z, wherein the bovine colostrum is skim bovine colostrum, late bovine colostrum partially defatted bovine colostrum, a mixture of bovine colostrum whey concentrate and whey protein concentrate, whole bovine colostrum, or a combination thereof.

In an embodiment BB, the composition of embodiment Y, wherein the isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II) Platelet-derived growth factor (PDGF), Vascular Endothelial Growth Factor (VEGF), Milk fat globule-epidermal growth factor 8 (MFG-E8), or nutraceutically acceptable salts thereof.

In an embodiment CC, the composition of any one of embodiments X to BB, wherein the stabilizing agent is selected from the group consisting of casein, caseinate salt, polydextrose, trehalose, exogenous colostrum fat, stearine, alginate, alginate plus ethyl cellulose aqueous dispersion, alginate plus ethyl cellulose, alginate plus maize starch, and alginate plus acrylic polymer.

In an embodiment DD, the composition of any one of embodiments X to CC comprising about 40 wt % to about 98 wt % colostrum, late colostrum, skim colostrum, partially defatted colostrum, or a mixture of colostrum whey concentrate and whey protein concentrate.

In an embodiment EE, the composition of claim DD, comprising about 2 wt % to about 40 wt % stabilizing agent.

In an embodiment FF, the composition of embodiment DD, further comprising soy lectin, medium-chain triglycerides, whey protein concentrate, non-fat dry milk, milk protein concentrate, or combinations thereof.

In an embodiment GG, the composition of claim FF, comprising up to about 2 wt % soy lecithin, up to about 2 wt % medium-chain triglycerides, up to about 2 wt % flavoring, up to about 50 wt % whey protein concentrate, up to about 10 wt % non-fat dry milk, up to about 10 wt % milk protein concentrate, or combinations thereof.

In an embodiment HH, the composition of embodiment DD, wherein the ratio of colostrum whey concentrate to whey protein concentrate is from about 1:20 to about 20:1.

In an embodiment II, the composition of embodiment DD, wherein the ratio of colostrum whey concentrate to whey protein concentrate is 60:40.

In an embodiment JJ, the composition of any one of embodiments DD to II, wherein the stabilizing agent is selected from the group consisting of calcium caseinate, sodium caseinate, and caseinate isolate from colostrum.

In an embodiment KK, the composition of embodiment JJ, comprising about 2 wt % to about 7 wt % calcium caseinate.

In an embodiment LL, the composition of any one of embodiments DD to KK, wherein the composition comprises a total protein content from about 50 wt % to about 80 wt % of the composition.

In an embodiment MM, the composition of any one of embodiments DD to KK, wherein the composition comprises immunoglobulin G in an amount from about 15 wt % to about 60 wt % of the composition.

In an embodiment NN, a dosage form comprising the composition of any one of embodiments A to MM.

In an embodiment OO, the dosage form of embodiment NN, wherein the form is a powder, granule, tablet, orally disintegrating tablet, capsule, acid resistant capsule, troche, lozenge, nutrition bar, soft chew, hydrogel, gummy, yogurt, suspension, or ready to drink beverage.

In an embodiment PP, the dosage form of any one of embodiments NN to LL, wherein the form is an acid resistant capsule.

Embodiment QQ is directed to a method of treating, alleviating, or preventing a disease or disorder associated with inflammation of the gastrointestinal tract in a subject in need thereof, comprising administering a composition to the subject, the composition comprising: a) a therapeutically effective amount of a pro-reparative factor, an immune factor, or a combination thereof; b) a nutritional protease inhibitor; and c) optionally a nutraceutically acceptable carrier or excipient.

In an embodiment RR, the method of embodiment QQ, wherein the composition further comprises a stabilizing agent.

In an embodiment SS, the method of embodiment RR, wherein the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, and alginate plus acrylic polymer, or a combination thereof.

In an embodiment TT, the method of any one of embodiments QQ to SS, wherein the pro-reparative factor is selected from a colostrum, late colostrum, skim colostrum, partially defatted colostrum, a mixture of colostrum whey concentrate and whey protein concentrate, an egg product, an isolated pro-reparative peptide or protein, or a nutraceutically acceptable salt thereof, or a combination thereof.

In an embodiment UU, the method of embodiment TT, wherein the colostrum is bovine colostrum.

In an embodiment VV, the method of embodiment UU, wherein the bovine colostrum is selected from the group consisting of skim bovine colostrum, late bovine colostrum, partially defatted bovine colostrum, a mixture of bovine colostrum whey concentrate and whey protein concentrate, partially defatted bovine colostrum, whole bovine colostrum, or a combination thereof.

In an embodiment WW, the method of embodiment TT, wherein the isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), Vascular Endothelial Growth Factor (VEGF), Milk fat globule-epidermal growth factor 8 (MFG-E8), or nutraceutically acceptable salts thereof.

In an embodiment XX, the method of embodiment TT, wherein the therapeutically effective amount of the isolated pro-reparative peptide or protein, or nutraceutically acceptable salt thereof, is administered at from about 0.001 mg/kg to about 10 mg/kg, about 0.005 mg/kg to about 5 mg/kg, about 0.01 mg/kg to about 1 mg/kg, or from about 20 μg/kg to about 200 μg/kg body weight of the subject per day.

In embodiment YY, the method of embodiment TT, wherein the therapeutically effective amount of the isolated pro-reparative peptide or protein, or nutraceutically acceptable salt thereof, is administered at from about 0.01 mg to about 500 mg, about 0.05 mg to about 80 mg, about 0.1 to about 50 mg, about 0.5 to about 10 mg, or from about 1 mg to about 5 mg per day.

In embodiment ZZ, the method of embodiment QQ, wherein the immune factor is selected from the group consisting of an immunoglobulin, cytokine, an anti-microbial peptide, or combinations thereof.

In an embodiment AAA, the method of embodiment QQ, wherein the nutritional protease inhibitor is casein.

In embodiment BBB, the method of embodiment QQ, wherein the nutritional protease inhibitor is a nutritional trypsin inhibitor.

In an embodiment CCC, the method of embodiment BBB, wherein the nutritional trypsin inhibitor is an enzyme active soy product, an ovomucoid, a soybean trypsin inhibitor, or a combination thereof.

In an embodiment DDD, the method of embodiment CCC, wherein the enzyme active soy product is a raw soy flour, raw soy meal, raw soy product, minimally processed soybean product, or a combination thereof.

In an embodiment EEE, the method of any one of embodiments QQ to DDD, wherein the composition comprises: about 40 wt % to about 95 wt % colostrum, late colostrum, skim colostrum, partially defatted colostrum, or a mixture of colostrum whey concentrate and whey protein concentrate; and about 5 wt % to about 25 wt % enzyme active soy product.

In an embodiment FFF, the method of embodiment EEE, wherein the composition comprises about 5 wt % to about 40 wt % stabilizing agent.

In an embodiment GGG, the method of embodiment EEE, wherein the composition comprises about 5 wt % to about 40 wt % casein.

In an embodiment HHH, the method of embodiment EEE, wherein the composition comprises about 2 wt % to about 7 wt % casein.

In an embodiment III, the method of embodiment EEE, wherein the composition further comprises soy lectin, medium-chain triglycerides, whey protein concentrate, non-fat dry milk, milk protein concentrate, or combinations thereof.

In an embodiment JJJ, the method of embodiment EEE, wherein the composition comprises up to about 2 wt % soy lecithin, up to about 2 wt % medium-chain triglycerides, up to about 2 wt % flavoring, up to about 50 wt % whey protein concentrate, up to about 10 wt % non-fat dry milk, up to about 10 wt % milk protein concentrate, or combinations thereof.

In an embodiment KKK, the method of embodiment EEE, wherein the ratio of colostrum whey concentrate to whey protein concentrate is from about 1:20 to about 20:1.

In an embodiment LLL, the method of embodiment EEE, wherein the ratio of colostrum whey concentrate to whey protein concentrate is 60:40.

In an embodiment MMM, the method of any one of embodiments YY to EEE, wherein the composition comprises a total protein content of the composition is from about 50 wt % to about 80 wt % of the composition.

In an embodiment NNN, the method of any one of embodiment EEE to MMM, wherein the composition comprises immunoglobulin G in an amount from about 15 wt % to about 60 wt % of the composition.

Embodiment OOO is directed to a method of treating, alleviating, or preventing a disease or disorder associated with inflammation of the gastrointestinal tract in a subject in need thereof, comprising administering a composition to the subject, the composition comprising: a) a therapeutically effective amount of a pro-reparative factor and/or an immune factor; b) a stabilizing agent; and c) optionally a nutraceutically acceptable carrier or excipient.

In an embodiment PPP, the method of embodiment OOO, wherein the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, and alginate plus acrylic polymer, or a combination thereof.

In an embodiment QQQ, the method of embodiment OOO, wherein the pro-reparative factor is selected from a colostrum, late colostrum, skim colostrum, partially defatted colostrum, a mixture of colostrum whey concentrate and whey protein concentrate, an egg product, an isolated pro-reparative peptide or protein, or a nutraceutically acceptable salt thereof, or combinations thereof.

In an embodiment RRR, the method of embodiment QQQ, wherein the colostrum is bovine colostrum.

In an embodiment SSS, the method of embodiment RRR, wherein the bovine colostrum is selected from the group consisting of skim bovine colostrum, late colostrum, a mixture of bovine colostrum whey concentrate and whey protein concentrate, partially defatted bovine colostrum, whole bovine colostrum, or a combination thereof.

In an embodiment TTT, the method of embodiment QQQ, wherein the isolated pro-reparative peptide or protein is a recombinant pro-reparative peptide or protein selected from the group consisting of Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), Vascular Endothelial Growth Factor (VEGF), Milk fat globule-epidermal growth factor 8 (MFG-E8), or nutraceutically acceptable salts thereof.

In an embodiment UUU, the method of embodiment TTT, wherein the therapeutically effective amount of the isolated pro-reparative peptide or protein, or nutraceutically acceptable salt thereof, is administered at from about 0.001 mg/kg to about 10 mg/kg, about 0.005 mg/kg to about 5 mg/kg, about 0.01 mg/kg to about 1 mg/kg, or from about 20 μg/kg to about 200 μg/kg body weight of the subject per day.

In an embodiment VVV, the method of embodiment TTT, wherein the therapeutically effective amount of the isolated pro-reparative peptide or protein, or nutraceutically acceptable salt thereof, or nutraceutically acceptable salt thereof, is administered at from about 0.01 mg to about 500 mg, about 0.05 mg to about 80 mg, about 0.1 to about 50 mg, about 0.5 to about 10 mg, or from about 1 mg to about 5 mg per day.

In an embodiment WWW, the method of embodiment OOO, wherein the immune factor is selected from the group consisting of an immunoglobulin, cytokine, an anti-microbial peptide, or combinations thereof.

In an embodiment XXX, the method of any one of embodiments QQ to WWW, wherein the disease or disorder is inflammatory bowel disease, non-steroidal antiinflammatory drug (NSAID) gastrointestinal disorder, chemotherapy-induced mucositis, radiation-induced mucositis, pseudomembranous colitis, gastritis, peptic ulcers, necrotizing entercolitis, irritable bowel syndrome, leaky gut, small intestinal bacterial overgrowth (SIBO), non-ulcer dyspepsia, or functional dyspepsia.

In an embodiment YYY, the method of embodiment XXX, wherein the inflammatory bowel disease is ulcerative colitis, indeterminate colitis, or Crohn's disease.

In an embodiment ZZZ, the method of any one of embodiment QQ to YYY, wherein the composition is effective to reduce myeloperoxidase levels in the subject.

In an embodiment AAAA, the method of any one of embodiments QQ to YYY, wherein the composition is effective to significantly preserve activity of the pro-reparative factor or immune factor when subjected to gastric juice or hydrochloride/pepsin digestion and chymotrypsin/trypsin digestion, when compared to activity of pro-reparative factor and or immune factor alone under the same conditions.

In an embodiment BBBB, the method of any one of embodiments QQ to YYY, wherein the method further comprises co-administering an effective amount of one or more additional active agents to the subject.

In an embodiment CCCC, the method of any one of embodiments QQ to BBBB, wherein the subject is a non-neonate human subject.

Embodiment DDDD is directed to a method of enhancing a vaccination antibody titer in a subject, comprising: a) orally administering a therapeutically effective amount of a composition to the subject, the composition comprising: i) a pro-reparative factor, an immune factor, or a combination thereof; ii) a nutritional protease inhibitor; iii) optionally a nutraceutically acceptable carrier or excipient; and iv) optionally a stabilizing agent; and b) administering a vaccine to the subject.

In an embodiment EEEE, the method of embodiment DDDD, wherein the vaccine is a COVID-19 vaccine, influenza vaccine, pneumococcal vaccine, tetanus vaccine, tetanus-diphtheria-pertussis vaccine, measles-mumps-rubella vaccine, human papillomavirus vaccine, hepatitis A vaccine, hepatitis B vaccine, meningococcal vaccine, Haemophilus influenzae type b vaccine, or zoster vaccine.

In an embodiment FFFF, the method of embodiment DDDD, wherein the vaccine is a veterinary vaccine, optionally wherein the veterinary vaccine is a distemper vaccine, rabies vaccine, parvovirus vaccine, avian influenza vaccine, equine West Nile virus vaccine, equine influenza virus vaccine, feline leukemia virus vaccine, lymphocytic choriomeningitis virus vaccine, bovine viral diarrhea virus vaccine, feline immunodeficiency virus vaccine, porcine herpesvirus vaccine, foot-and-mouth disease virus vaccine, bovine herpesvirus-1 related disease vaccine, or Newcastle disease virus vaccine.

In an embodiment GGGG, a dietary supplement comprising the composition of any one of embodiments A to MM.

Embodiment HHHH is directed to a composition comprising a purified lactoferrin, a stabilizing agent, and optionally a nutraceutically acceptable carrier or excipient.

In embodiment IIII, the composition of embodiment HHHH, wherein the stabilizing agent is casein, caseinate salt, polydextrose, trehalose, exogenous colostrum fat, stearine, alginate, alginate plus ethyl cellulose aqueous dispersion, alginate plus ethyl cellulose, alginate plus maize starch, or alginate plus acrylic polymer.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

### EXAMPLES

### Example 1. Nutritional Serine Protease Inhibitors Stabilize Growth Factor Activity of Pro-Reparative Factors

In this series of experiments, it was examined whether administration of the nutritional protease inhibitors ovomucoid (present in egg) or soya bean trypsin inhibitor (SBTI, from soy) were able to reduce the destruction of growth factor biological activity present in bovine colostrum (BC) and/or egg, due to pancreatic enzymes. In addition, it was examined whether proliferative bioactivity could be preserved when using recombinant epidermal growth factor (EGF) as an example of a growth factor present in BC. An in vivo rat DSS-induced colitis model was used to examine whether ovomucoid or SBTI given alone could truncate DSS-induced injury (through preserving endogenous luminal growth factors) and whether they could enhance the protective effect of orally administered BC±egg or EGF.

### Materials and Methods

Pasteurized BC powder and a commercial chicken whole egg powder were provided by Pantheryx Inc. (Boulder, Colo., USA). Colostrum powder was collected within the first 24 hours after calving and subsequent powder comprised 48.32 g protein, 16.25 g fat, 24.53 g carbohydrate, 4.5 g moisture, and 6.4 g ash per 100 g of powder. Egg powder comprised 50.45 g protein, 42.71 g fat, 1.25 g carbohydrate, 1.72 g moisture, and 3.87 g ash per 100 g. The BC+egg combination was used in a wt ratio of 40:60.

Sources of other peptides were as follows: Human EGF (Peprotech, New Jersey, USA), SBTI (Roche, Calif., USA), Ovomucoid (Sigma, USA), and BSA (Sigma). AGS cell line is derived from gastric adenocarcinoma of a 54-year-old female (ATCC, LGC Standards, UK).

Study 1. Effect of Protease Inhibitors on Growth Factor Preservation In Vitro.

Digestion of Samples

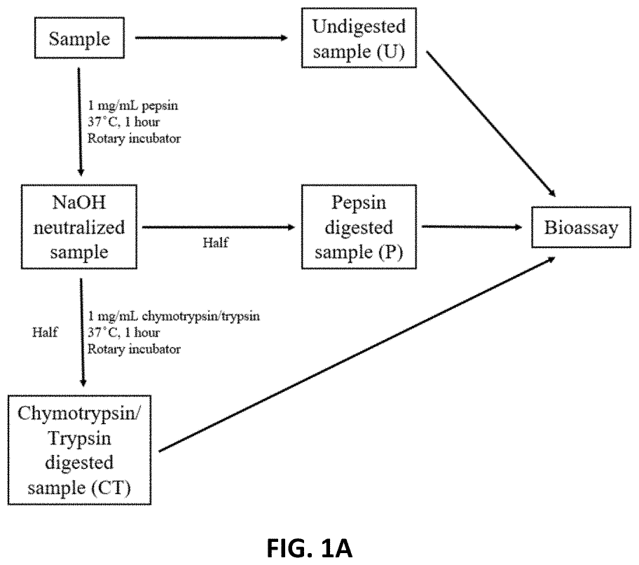

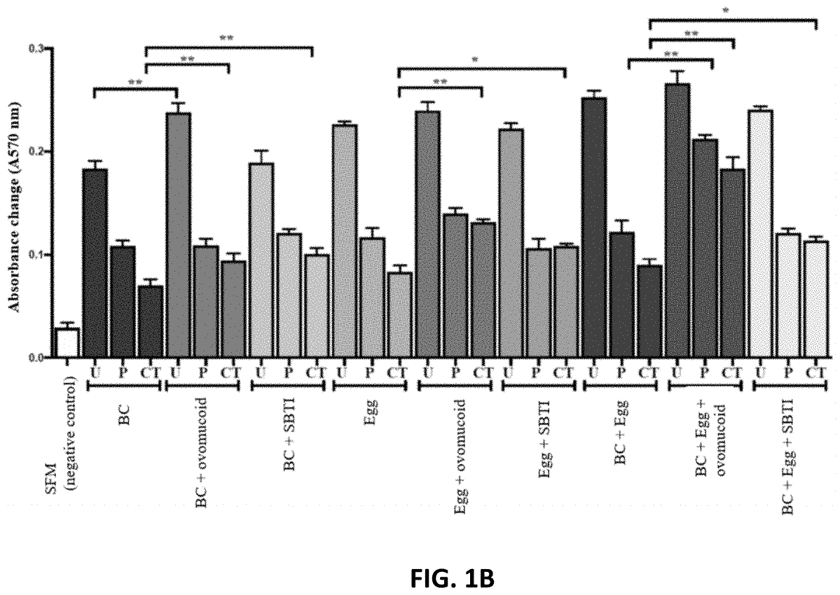

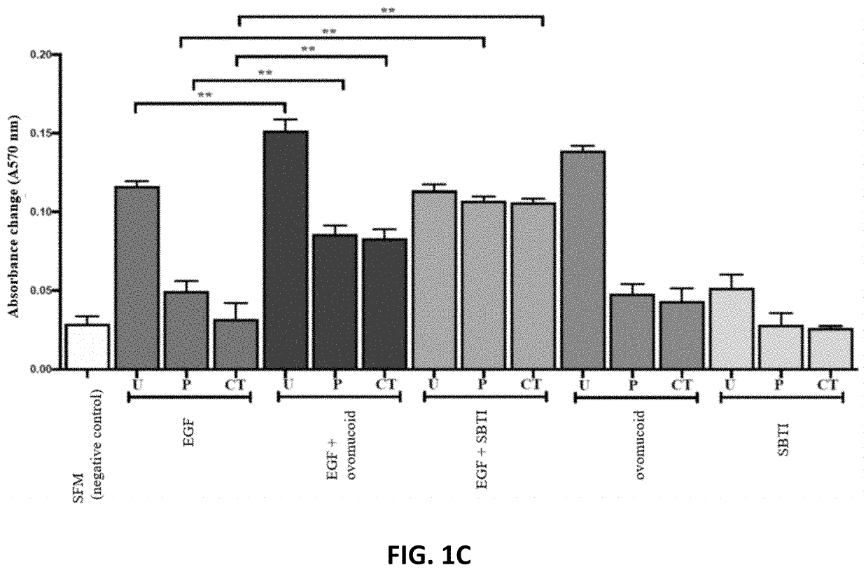

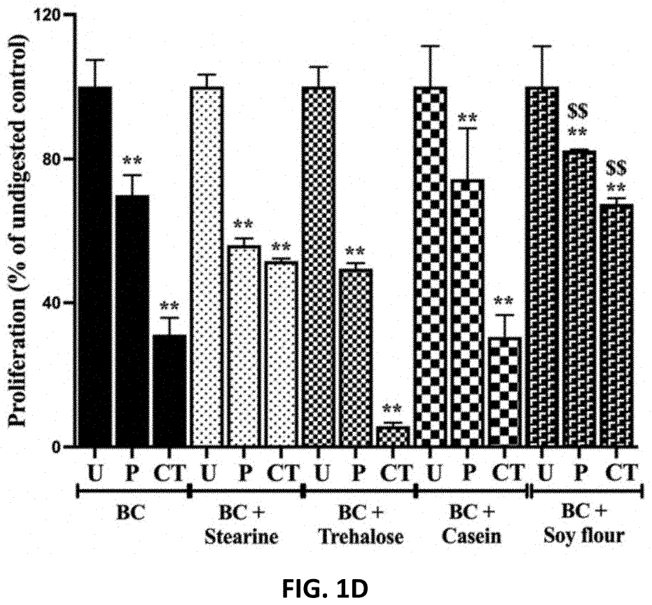

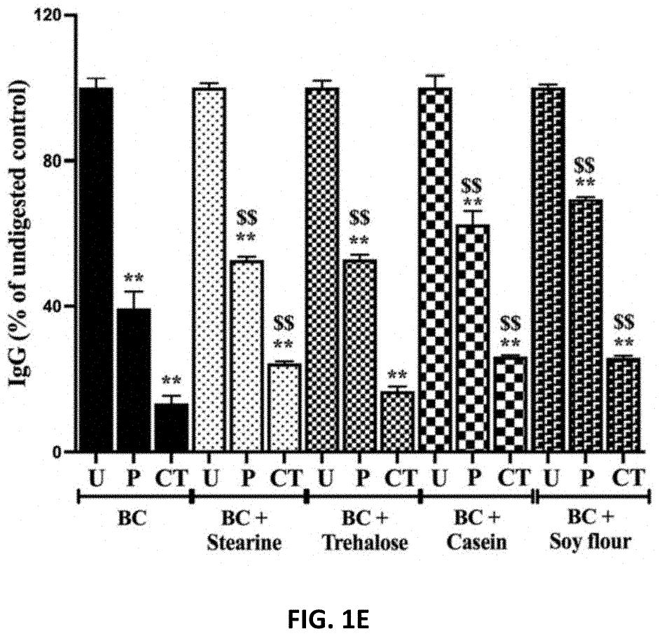

To reproduce intraluminal exposure of peptides within the stomach and small intestine, samples were tested undigested, following exposure to HCl/pepsin alone (1h) or HCl/pepsin (1h) followed by chymotrypsin and trypsin exposure (1h), using the protocol shown in FIG. 1A. Briefly, aliquots of BC alone (400 mg), egg alone (400 mg), BC+egg (400 mg total weight in ratio 60:40) and EGF (100 μg) were made up in 10 ml of PBS in the presence or absence of ovomucoid (500 mg) or SBTI (1 g), incubated at 37° C. without addition of pepsin, chymotrypsin or trypsin (undigested control), or incubated in pepsin (1 mg/ml) pH2 in a rotary incubator at 37° C. for 1 hour followed by neutralization to pH7 using NaOH. Half of the aliquot was then removed (pepsin digested sample) and the reminder incubated in chymotrypsin and trypsin (1 mg/ml) for 1 hour (chymotrypsin/trypsin digested sample). Additional samples examined the susceptibility of ovomucoid or SBTI alone to digestion. All samples were subsequently analyzed for proliferative bioactivity in the same assay.

Proliferation Assays

Cell proliferation assays were performed using Alamar blue (Invitrogen, Paisley, UK), as per manufacturer's instructions. Briefly, cells were seeded at 2000 cells/well, grown in DMEM medium and 10% FCS in 96 well plates overnight. The following day, cells were washed with serum free medium (SFM) and incubated in SFM alone (negative control), or in the presence of undigested or digested samples diluted to identical final concentrations. BC±egg constituents were tested at 1 mg powder/ml, ovomucoid at 1.25 mg/ml, EGF at 0.25 μg/ml and SBTI at 2.5 mg/mL. These concentrations were based on preliminary dose-response comparisons.

Results

Study 1. Effect of Protease Inhibitors on Growth Factor Preservation In Vitro.

Similar results were seen using bovine colostrum (BC) alone or egg alone; exposure to HCl/pepsin reduced bioactivity by about 50% with a further 15% reduction following CT exposure (FIG. 1B). Co-presence of ovomocoid or SBTI did not influence HCl/pepsin susceptibility but reduced CT digestion by about 80% (all P<0.01). BC+egg showed a similar susceptibility to HCl/pepsin and CT digestion as incubating the individual factors alone. Co-presence of SBTI did not affect peptic HCl/pepsin susceptibility but reduced CT sensitivity. Co-presence of ovomucoid reduced both. Nutritional protease inhibitors ovomucoid or SBTI significantly protected the proliferative activity of BS, egg, or BC+egg from CT digestion. Ovomucoid also significantly protected proliferative activity of BC+egg from P digestion.

EGF incubated alone with HCl/pepsin caused a 76% reduction in bioactivity with a further reduction of 20% following incubation with CT (FIG. 1C). Co-presence of ovomucoid did not affect HCl/pepsin susceptibility but significantly reduced the loss of bioactivity caused by CT. Co-presence of SBTI reduced both HCl/pepsin and CT loss of biological activity. SBTI alone did not stimulate proliferation when given alone. Ovomucoid alone stimulated proliferation but this was completely lost following HCl/pepsin digestion. Nutritional protease inhibitors ovomucoid or SBTI significantly protected proliferative activity of EGF from both P and CT digestion.

Study 2. Rat DSS Colitis Model

Animal studies were performed within BolderBiopath Laboratories, Boulder, Co, USA in accordance with the commercial test facility standard operating procedures and license, the World Health Organization Quality Practices in Basic Biomedical Research guidelines, and in compliance with all state and federal regulations, including USDA Animal Welfare Act 9 CFR Parts 1-3. Federal Register 39129, Jul. 22, 1993.

Female Sprague-Dawley rats (174-204 g) were obtained from Envigo RMS, Inc. (Indianapolis, USA), housed in standard cages (five per cage) and fed Envigo Teklad 8640 diet and tap water ad libitum. Methods used for the rat dextran sulfate sodium (DSS) colitis model were as described by Playford, R. J., et al. “Pasteurized Chicken Egg Powder Stimulates Proliferation and Migration of AGS, RIE1, and Caco-2 Cells and Reduces NSAID-Induced Injury in Mice and Colitis in Rats,” The Journal of Nutrition, 2020, 150, 1434-1442. Eight groups were tested (N=10 per group). All rats received a single 1 ml oral gavage (made up in 3% sodium bicarbonate to neutralize gastric acidity) daily for 9 days. Negative control group received no DSS but received daily gavage with bovine serum albumin (20 mg/kg, BSA). Positive control group received DSS and gavage with 20 mg/kg BSA. The remaining 6 groups received BC+egg (40:60 ratio, 20 mg/kg), EGF (100 μg/dose), ovomucoid (5000 μg/dose), or SBTI (10.8 mg/dose) alone. The final two groups comprised the combination of EGF+SBTI or BC+egg+ovomucoid. These groups were chosen based on the results of study 1 as optimal protective effects were seen with ovomucoid for BC+egg and SBTI for EGF.

Colitis was induced by adding 5% (w/v) DSS (molecular mass, 40-50 kDa; Spectrum, catalogue No. DE136, lot No. 1IG1103) to the drinking water for 7 days, starting from day 3 of the test product gavage period. Mean DSS and food consumption were noted per cage each day. Rats were weighed daily and visually inspected for signs of distress, diarrhea, and rectal bleeding. The disease activity index (DAI, based on Cooper, H. S., et al“Clinicopathologic Study of Dextran Sulfate Sodium Experimental Murine Colitis,” Lab Invest, 1993, 69, 238-49) was assessed daily following induction of colitis. The DAI combines the scores of weight loss, stool consistency, and bleeding divided by 3. A cumulative score was then determined over the 7-day DSS treatment period.

At the end of the study, rats were sacrificed, and colonic tissue was collected for biochemical and histopathological assessment. Microscopic damage was assessed using scoring system described Williams, K. L. et al., “Enhanced Survival and Mucosal Repair after Dextran Sodium Sulfate-Induced Colitis in Transgenic Mice that Overexpress GrowthHhormone,” Gastroenterology, 120, 925-37 (2001). The total histological colitis score is derived from the sum of the four subscores of i) inflammation severity, ii) inflammation extent, iii) crypt damage, and iv) percentage of involvement. Tissue was also analyzed for myeloperoxidase (MPO) activity (used as a marker of neutrophilic infiltration).

Statistics

All results are expressed as mean+/−SEM. Statistics were performed using Graphpad Prism 8 version 8.3.1. Test for normality of data using Shapiro Wilks test showed equal variances between groups. Proliferation and in vivo studies were analysed using one-way analysis of variance (ANOVA). When a significant effect was seen (P<0.05), individual comparisons were performed with the use oft tests based on group means, residual, and degrees of freedom obtained from the ANOVA, a method equivalent to repeated-measures analyses.

Results

Study 2: Rat DSS Colitis Model.

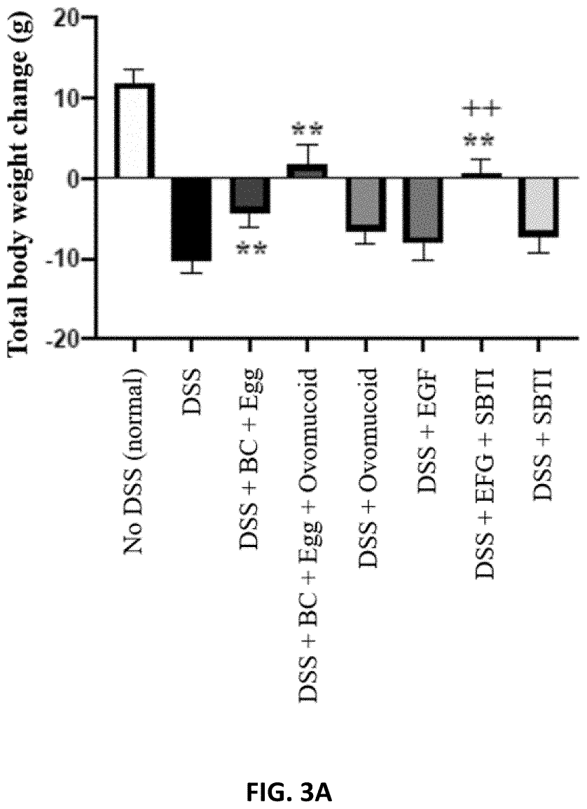

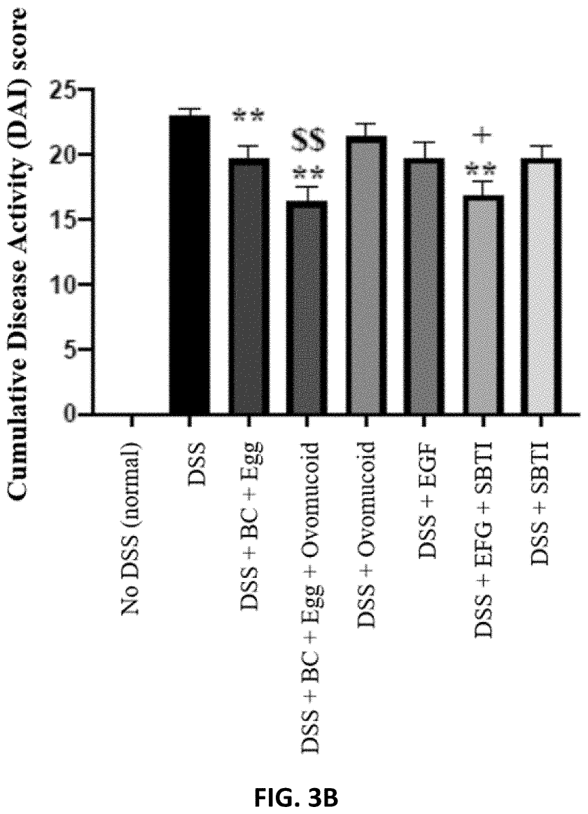

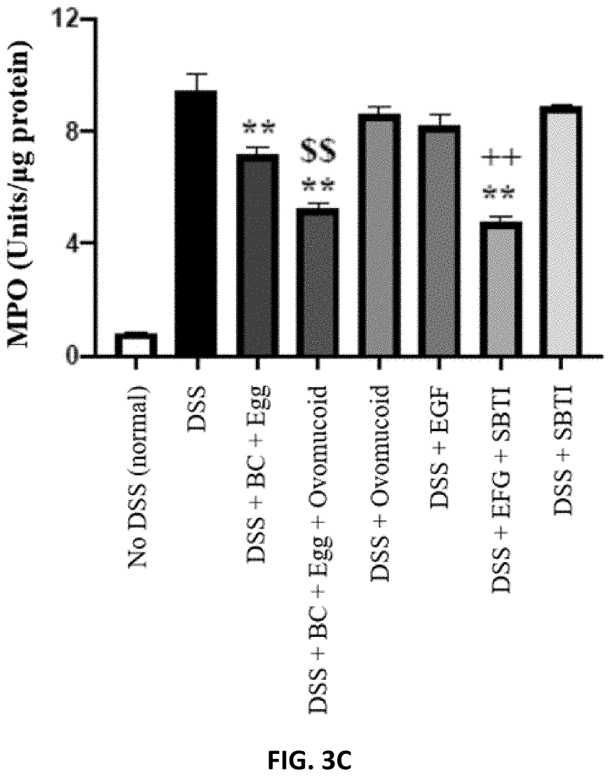

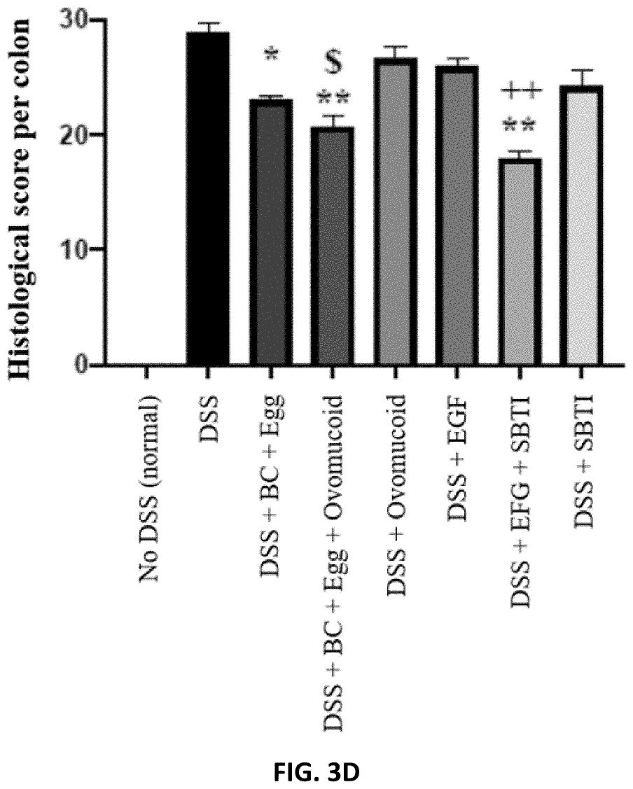

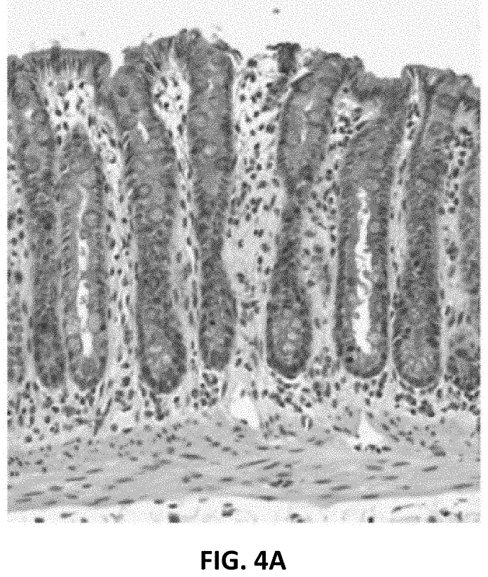

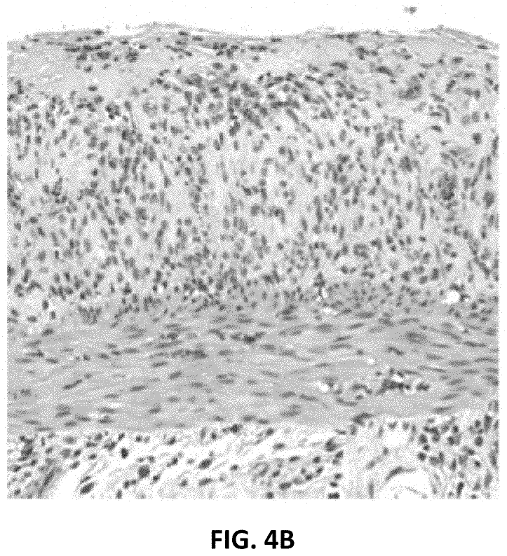

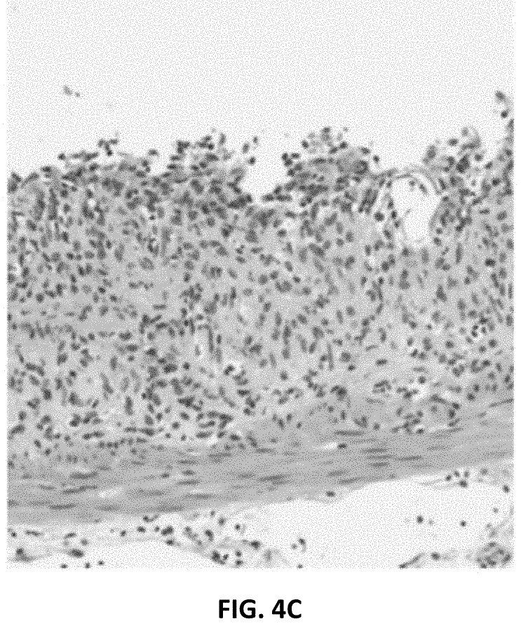

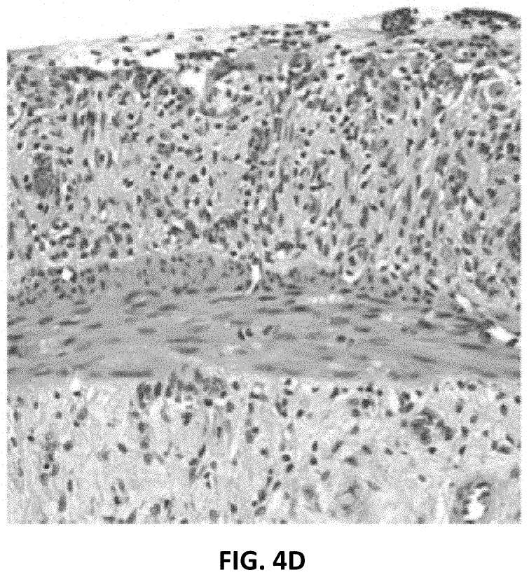

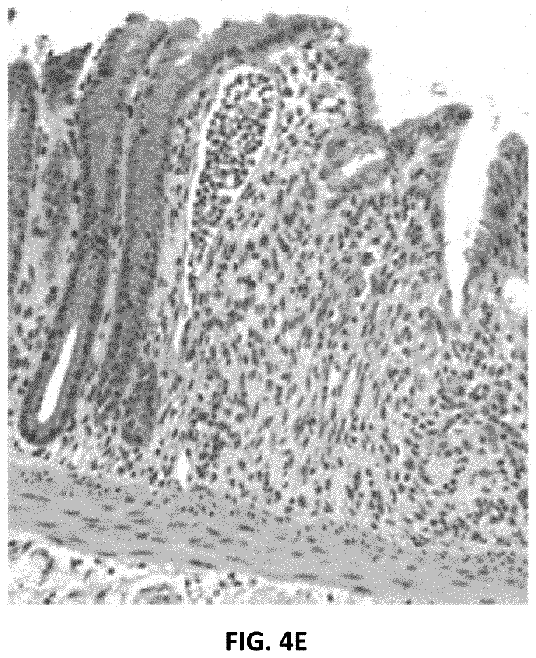

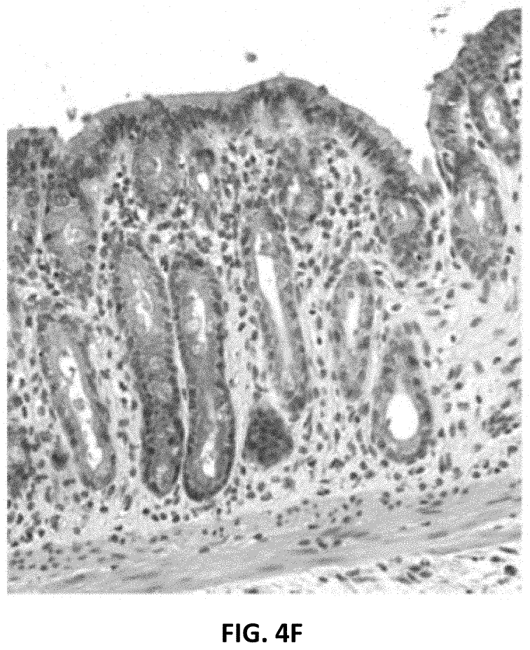

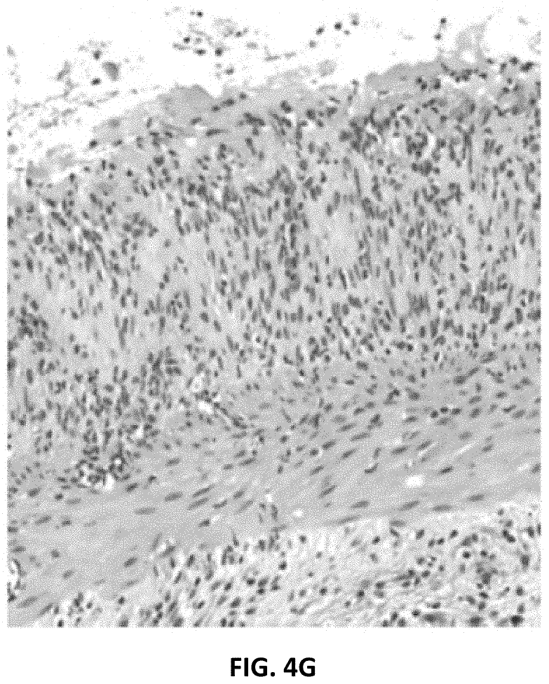

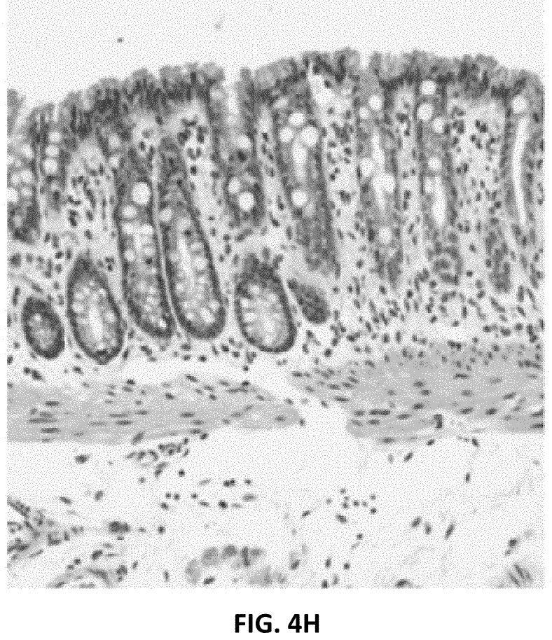

Rats that did not receive DSS showed an increase in total body weight of 11.8+/−1.9 g over the 9-days (FIG. 3A). In contrast, animals that received DSS alone showed marked weight loss (−10.2+/−1.5 g)(P<0.01 compared with no DSS) over the same period. Administration of ovomucoid alone to DSS treated animals did not significantly affect weight loss, whereas BC+egg significantly truncated weight loss induced by DS S (P<0.01 vs DSS alone). The most improvement in weight was seen in animals given BC+egg+ovomucoid together (P<0.01). EGF or SBTI given alone did not affect the weight loss induced by DSS. In contrast, the combination of EGF+SBTI showed marked improvement in weight compared to DSS alone P<0.01).

Analyses of cumulative Disease Activity Index (DAI) scores as shown in FIG. 3B gave similar results to those seen following weight changes of animals. Ovomucoid given alone did not affect DAI scores. Beneficial effects were seen using BC+egg and the best result was seen using the combination of BC+egg+ovomucoid (P<0.01 versus DSS alone or BC+egg in DSS treated animals). EGF or SBTI given alone did not affect DSS-induced changes in DAI whereas the combination significantly reduced DAI scores (P<0.01).

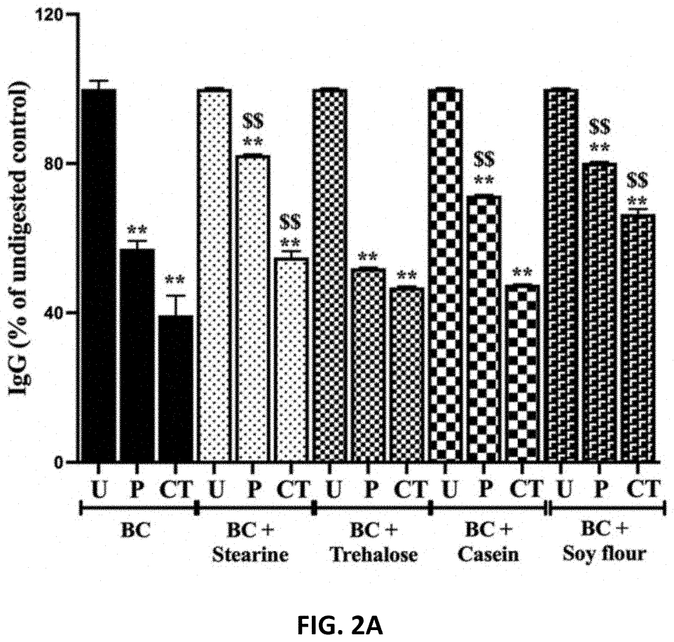

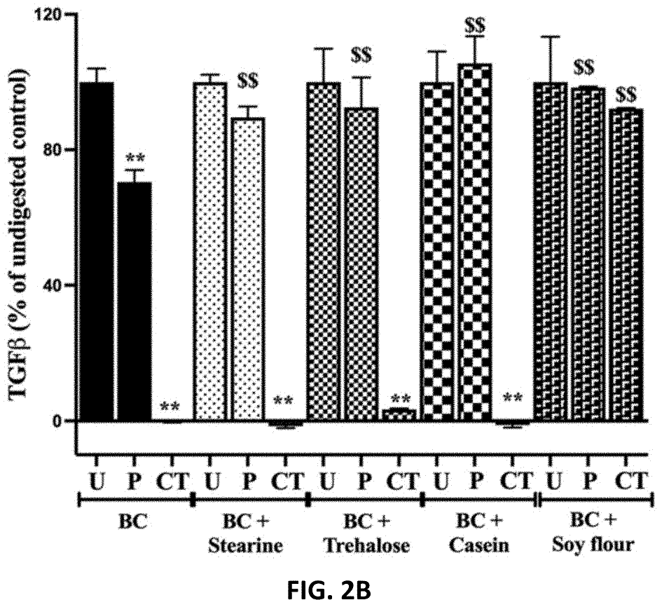

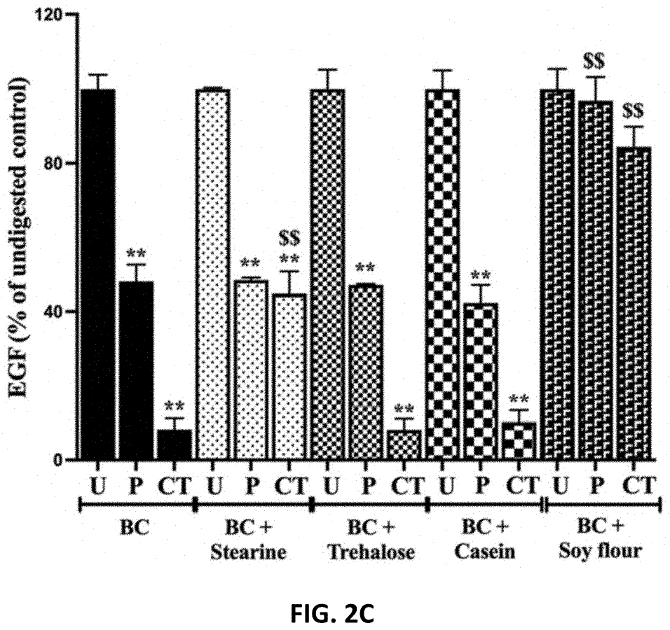

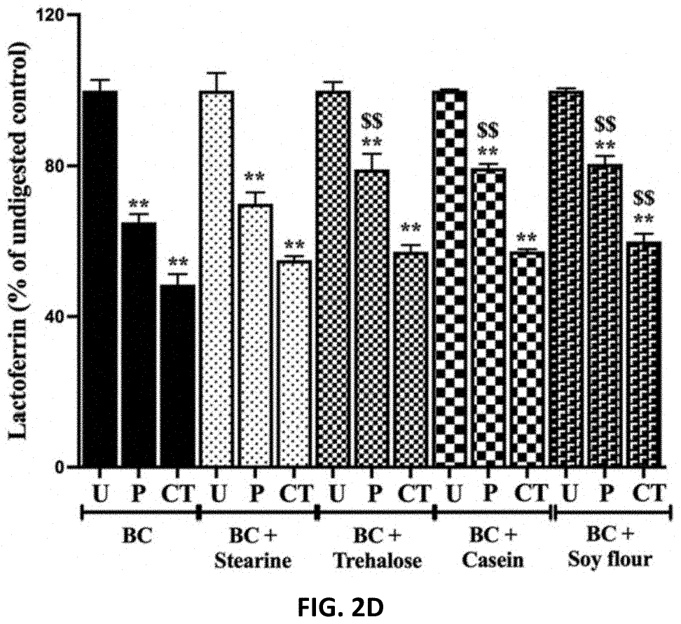

Results from myeloperoxidase (MPO) analyses as shown in FIG. 2C were in keeping with weight change and DAI scores. MPO is a marker of neutrophilic infiltration and can indicate immune activation or infection. Administration of DSS alone caused a 13-fold increase in colonic MPO. Administration of ovomucoid alone to DSS treated animals did not improve MPO levels, whereas BC+egg truncated the rise in MPO concentrations caused by DSS (P<0.01). Additional benefit was seen if the BC+egg was co-administered with ovomucoid (P<0.01 vs DSS alone, DSS+ovomucoid or DSS+BC+egg). EGF or SBTI given alone did not affect DSS-induced changes in MPO whereas the combination significantly reduced MPO levels (P<0.01).

Morphology showed that compared with normal (no DSS) controls (FIG. 3A), administration of DSS caused almost complete loss of normal crypt structure combined with major infiltration of inflammatory cells (FIG. 3B). Treatment with ovomucoid alone (FIG. 3C), SBTI (FIG. 3D) or EGF alone (FIG. 3G) did not influence damaging effect of DSS. Improvement was seen in animals that received BC+egg, with the inflammatory infiltrate being less marked and crypt structure partially maintained (FIG. 3E). Animals that received the combination of BC+egg+ovomucoid (FIG. 3F) or EGF+SBTI (FIG. 3H) showed major improvement with minimal inflammatory infiltrate, and maintenance of crypt structures. Formal histological scoring (FIG. 2D) showed similar results to that seen on morphology; No beneficial effect was found using ovomucoid alone, SBTI alone or EGF alone. Significant improvement was seen using BC+egg (P<0.05 vs DSS alone), with the greatest improvement seen in animals that had received either BC+egg+ovomucoid or EGF+SBTI (P<0.01 vs DSS alone).

The present example supports the conclusion that compositions and methods of the disclosure can have value for multiple GI conditions including NSAID-induced small intestinal gut injury (as acid suppressants do not influence SI damage), UC (colonic injury) and Crohn's disease (which predominantly affects the terminal small intestine).

### Example 2. Enzyme Active Soy Flour with Colostrum Synergistically Decreases Disease Activity Index and Reduces Colonic Inflammatory Activity In Vivo

An animal model of Dextran Sodium Sulphate (DSS)-induced colitis in mice was employed to determine effect of colostrum and/or soy flour on Disease Activity Index (DAI), and to determine DAI score following 9 days of test product, with 7 days of DSS (starting on day 3) to cause colitis. In this assay, the colostrum was 60-20 skim primary colostrum (APS 60-201 skim colostrum powder, APS BioGroup), and the soy was an enzyme active full fat raw soy flour (EASY100, Natural Products, Inc., Grinnell, Iowa).

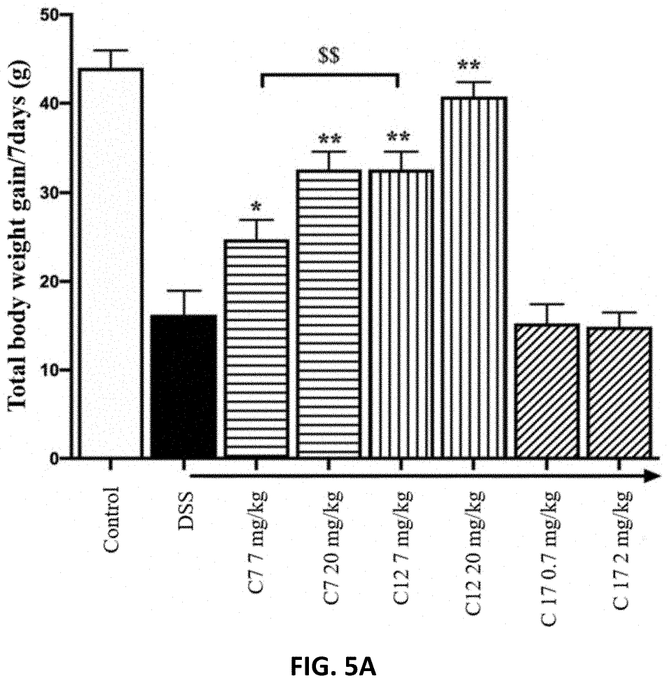

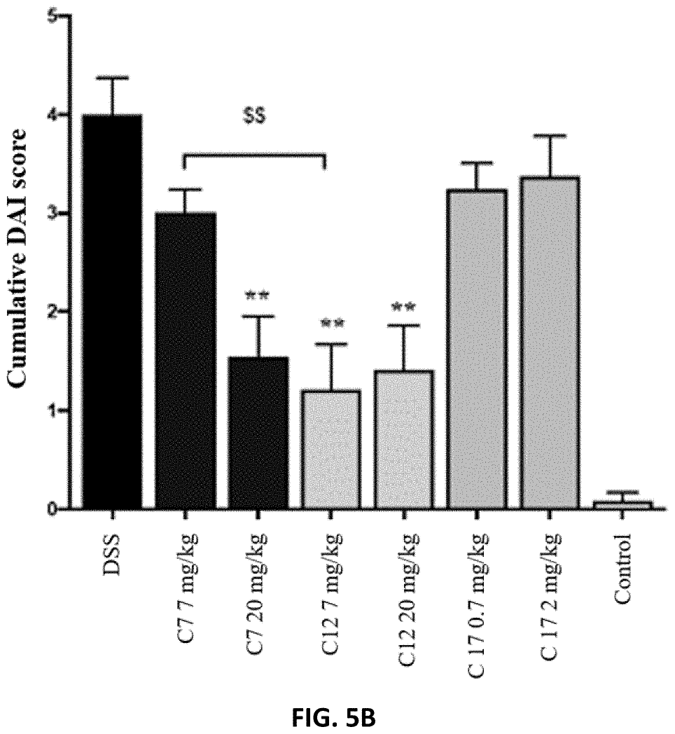

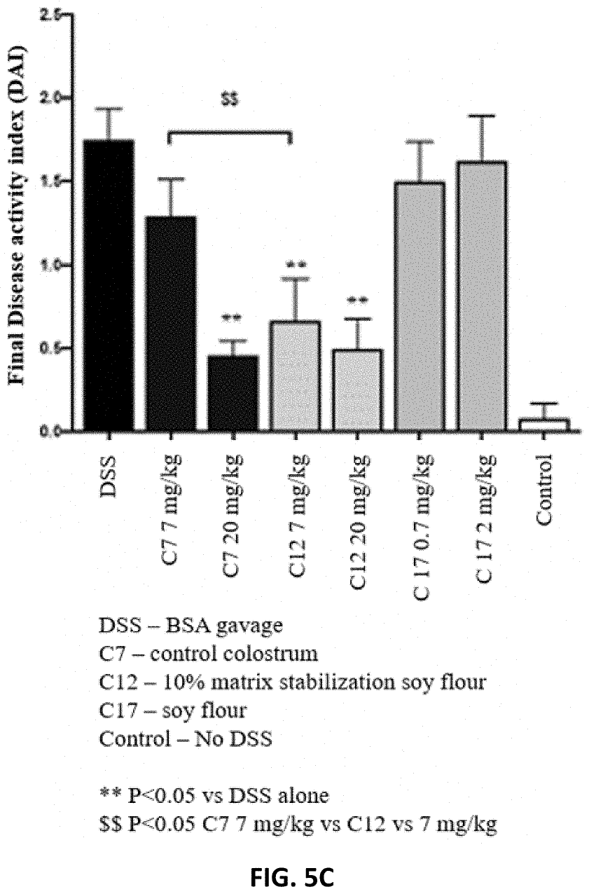

In this assay, Disease Activity Index (DAI) scores were determined for mice receiving normal (control), DSS alone (to cause colitis), and standard colostrum (C7) at a dose of 20 mg/kg and 7 mg/kg to show a dose response. Soy flour alone (C17) was tested at 0.7 mg/kg and 2 mg/kg. A mixture of colostrum containing 10% soy flour at the same doses (C12 at 7 and 20 mg/kg) was tested. Results are shown in FIG. 5A thru FIG. 5C. FIG. 5A shows total body weight gain over the 7 days of co-treatment with DSS. FIG. 5B shows cumulative Disease Activity Index (DAI) score, and FIG. 5B shows final DAI. The y axis of FIG. 5C is Disease Activity Index score (a standard measurement of health of the animals). Treatment with DSS dramatically slowed body weight gain of the mice during the 7 day DSS treatment (FIG. 5A). Both the colostrum compositions (C7) and the colostrum+soy flour compositions (C12) were able to rescue the DSS induced reduction in weight gain. Surprisingly, the C12 compositions performed significantly better that the C7 compositions at equivalent colostrum doses. With respect to cumulative DAI score and final DAI, it was surprisingly found that the low dose of the mixture of colostrum containing 10% soy flour (C12 6.3 mg/kg colostrum+0.7 mg/kg soy flour) was as effective as the higher dose, and as effective as the higher dose of colostrum C7 alone (colostrum 20 mg/kg) as shown in FIGS. 5A and 5B. Therefore, efficacy was three-fold higher (C12 at 7 mg/kg versus C7 at 20 mg/kg) using the combination rather than colostrum alone (see stats with $$ signs, P<0.05). The C17 columns show giving soya alone (C17) at the same amount as is in the colostrum+soya combination (i.e., 0.7 and 2 mg/kg) and which showed no real effect on preventing the DSS induced reduction in weight gain or the DAI assessments. Therefore, a synergistic response of the combination of colostrum+10% soy flour is demonstrated with respect to weight gain and Disease Activity Index (DAI) scores.

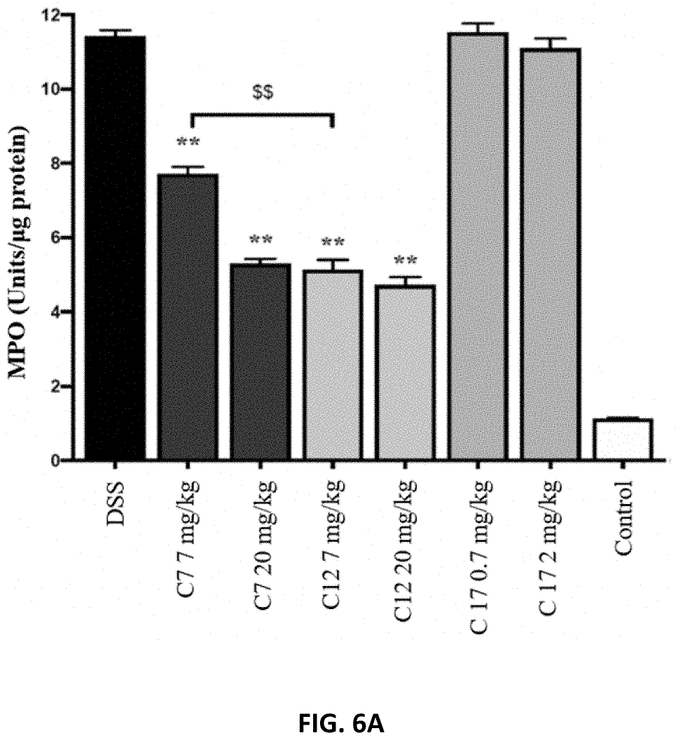

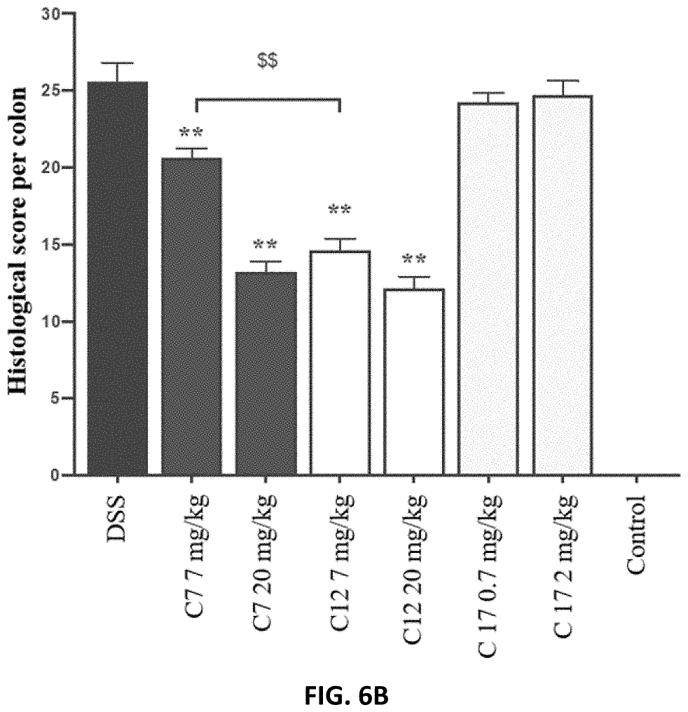

The myeloperoxidase (MPO) activity in colonic tissue of the mice was also measured as an indication of inflammation. As shown in FIG. 6A, C7 (colostrum alone) shows a dose response in ability to reduce inflammation (MPO levels). C17 soya alone shows no effect. C12 colostrum+soy shows significantly improved effect with the dose of 7 mg/kg of the combo being as effective as 20 mg/kg of colostrum alone, i.e., a three-fold enhancement. Therefore, a synergistic response is demonstrated with respect to reduction of colonic inflammatory activity when using a pro-reparative factor (colostrum) with a nutritional serine protease inhibitor (enzyme active soy flour).

FIG. 6B shows a bar graph of histological score per colon in DSS-induced colitis model of FIG. 6A, with same variables and statistics. No DSS shows normal histology and a zero damage score as expected. DSS alone shows virtually complete loss of normal architecture and major influx of inflammatory cells. This gives a high score on the graph. C17 (which is soy alone) does not improve damage on histology or histology scoring. C7 colostrum alone shows a good dose response with some improvement on the 7 mg/kg dose and a greater improvement when given at 20 mg/kg as reflected in histology scoring graph. C12 colostrum+soya shows a very good protective effect with both the 7 mg/kg and 20 mg/kg giving a much improved appearance. Formal scoring shows both 7 mg/kg and 20 mg/kg doses gave a significantly improved score compared to DSS alone. The dose of 7 mg/kg combination gave a similar appearance to the 20 mg/kg colostrum alone, therefore showing a roughly three-fold increase in bioactivity on a wt/wt basis. These findings are in keeping with the results of MPO shown in FIG. 6A.

Representative photomicrographs in DSS-induced colitis model are shown in FIG. 7A through FIG. 7G. FIG. 7A shows a representative photomicrograph in colonic tissue of the mice in DSS-induced colitis model for the No DSS control with normal histology and a zero damage score as expected. FIG. 7B shows a representative photomicrograph in colonic tissue of the mice in DSS-induced colitis model for DSS alone with virtually complete loss of normal architecture and major influx of inflammatory cells. FIG. 7C shows a representative photomicrograph in colonic tissue of the mice in DSS-induced colitis model for DSS+C7 colostrum at 7 mg/kg dose showing some improvement. FIG. 7D shows a representative photomicrograph in colonic tissue of the mice in DSS-induced colitis model for DSS+C7 colostrum at 20 mg/kg dose showing greater improvement than the 7 mg/kg dose. FIG. 7E shows a representative photomicrograph in colonic tissue of the mice in DSS-induced colitis model for C12 colostrum+soya shows a very good protective effect with the 7 mg/kg giving a much improved appearance. FIG. 7F shows a representative photomicrograph in colonic tissue of the mice in DSS-induced colitis model for C12 colostrum+soya shows a very good protective effect with the 20 mg/kg also giving a much improved appearance. FIG. 7G shows a representative photomicrograph in colonic tissue of the mice in DSS-induced colitis model for C17 (which is soy alone) does not improve damage on histology or histology scoring compared to DSS alone.

### Example 3. Soybean Trypsin Inhibitor Activity Assay

In order to determine trypsin inhibitor activity (TIA) in a soy product, a soybean trypsin inhibitor assay may be performed according to the method of Liu, K., “Soybean Trypsin Inhibitor Assay: Further Improvement of the Standard Method Approved and Reapproved by American Oil Chemists' Society and American Association of Cereal Chemists International,” J Am Chem Soc, 96:635-645 (2019).

Materials

Crystalline bovine trypsin (essentially salt-free, lyophilized powder), synthetic substrate N-α-benzoyl-DL-arginine-p-nitroanilide hydrochloride (DL-BAPA), Tris base (hydroxymethyl) amino-methane, and calcium chloride dehydrate are purchased from Sigma-Aldrich (St. Louis, Mo., USA). Deionized water is used throughout the assay.

Reagents

The assay buffer (50 mM Tris buffer, pH 8.2, containing 20 mM CaCl₂)) is made by dissolving 6.05 g Tris base and 2.94 g calcium chloride dihydrate in 900 mL water, adjusting to pH 8.2 and diluting to 1000 mL. The assay buffer is used for making BAPA working solution. The HCl solution (1 mM, containing 5 mM CaCl₂)) is used for making trypsin stock and working solutions. Acetic acid solution (30% v/v), used for stopping the trypsin reaction, is made by mixing 30 mL of glacial acetic acid with 70 mL water. BAPA stock solution (40 mg mL⁻¹): dissolve 400 mg of DL-BAPA in 10 mL dimethyl sulfoxide. The solution is very stable even at room temperature. BAPA working solution (0.4 mg mL⁻¹): dilute 0.5 mL of the BAPA stock solution to a total volume of 50 mL, using the assay buffer prewarmed at 37° C. Fresh BAPA working solution is prepared daily for the TIA assay. Trypsin stock solution (0.2 mg mL⁻¹): dissolve 10 mg of bovine trypsin in 50 mL of the above HCl solution, pH about 2.5. The solution is kept at 5° C. Trypsin working solution (20 μg mL⁻¹): dilute 2.5 mL of the trypsin stock solution to 25 mL with the HCl solution.

The soy product sample such as a soy flour, concentrate, or isolate, is passed through a US standard 100 sieve (equivalent to 150 um diameter sieve openings). One gram of the sample is extracted with 50.0 mL of 10 mM NaOH in a beaker using a magnetic stirrer at a low setting for 1 hour. For raw, boiled, oven-roasted soybeans, may be ground in a coffee grinder, passed through US standard No. 50 sieve (300 um diameter sieve openings), and extracted as above for 3 h. The sample suspension is diluted so that 2.0 mL of a dilute sample caused 30-70% trypsin inhibition after blank reading correction.

In a set of test tubes (e.g., 16 mL centrifuge tubes), the reagents are added according to the order of: dilute soy extract (2 mL, vary in mg/mL), DL-BAPA (5 mL, 0.4 mg/mL), and Trypsin (2 mL, 20 ug/mL). The whole assay is run in a water bath at 37° C. Exactly 10 min after adding the trypsin solution with immediate mixing, the color reaction is stopped by adding 1.0 mL 30% acetic acid solution. The mixture is centrifuged or filtered for clarification. The absorbance for the sample reading (A410_{S}) at 410 nm is a measure of the trypsin activity in the presence of the sample inhibitors. Concurrently, the reaction is also run in the absence of inhibitors by replacing the sample extract with an equal amount of water. The corresponding absorbance is the reference reading (also known as the standard reading in some other literature), symbolized as A410_{R}. In addition, reagent blanks for the sample readings (known as the sample blank, A410_{SB}) and a reagent blank for the reference readings (known as the reference blank, A410_{RB}) are also run by adding the acetic acid solution before the trypsin solution. For all absorbance readings, deionized water was used as a reference. Sample readings and reference readings are run in duplicate.

Calculation of TIA values may be performed under the conditions of the 10 mL assay, where a trypsin unit (TU) is defined as an increase of 0.01 absorbance at 410 nm. The trypsin inhibitory activity is expressed in trypsin units inhibited (TUI) or trypsin inhibitor units (TIU) per mg sample and calculated as follows: TUI per mg sample={[(A_{410R}−A_{410RB})−(A_{410S}−A_{410SB})×100]×mL diluted soy extract}/(mg sample per mL diluted soy extract used for the assay).

Typical trypsin inhibitor activity in several soy products was determined by Liu 2019, where 1 TU=0.01 A410, as follows. Soybeans (raw) 46.84±0.06 TUI/mg sample; Soy flour (enzyme active, full-fat) 48.24±0.14 TUI/mg sample; Soy flour (defatted, PDI=90) 65.70±0.14 TUI/mg sample; Soy flour (defatted, PDI=70) 49.46±0.17 TUI/mg sample; Soy flour (low fat) 34.28±0.19 TUI/mg sample; Soybeans (oven roasted) 6.30±0.22 TUI/mg sample; Soybeans (boiled) 3.18±0.18 TUI/mg sample; Soy flour (roasted, full fat) 9.68±0.32 TUI/mg sample; Soy flour (defatted, toasted, PDI=20) 11.99±0.23 TUI/mg sample; soy protein concentrate 9.46±0.20 TUI/mg sample; Soy protein isolate 8.03±0.36 TUI/mg sample. PDI=protein dispersibility index. TUI=trypsin units inhibited.

In some embodiments, the enzyme active soy product has a trypsin inhibitor activity greater than 10 TUI/mg, greater than 20 TUI/mg, greater than 30 TUI/mg, or greater than 40 TUI/mg when assayed according to the method of example 3, or Liu 2019. In some embodiments, the soy product has a PDI greater than 50, greater than 60, or greater than 70 when tested according to AOCS) Standard Procedure Ba 10b-09.

### Example 4. Prototype Colostrum Compositions

Prototype stabilized colostrum compositions were prepared using various stabilizing agents for either super agglomeration, microencapsulation, or matrix stabilization. Commercial 60201 (instantized) bovine skim colostrum was employed in Control composition A1 having 1.8% total fat by AOAC 932.06, 64% protein (dry basis) by AOAC991.20, and 31.6% IgG (HPLC) protein G (dry basis) by AOAC 2010.01. Prototype compositions are shown in Tables 2-4, respectively, utilizing the same colostrum with various additional components.

The A2-A8 prototypes shown in Table 2 were made by modifying the normal agglomeration step used to process colostrum. The agglomeration process is performed to instantize, to ensure particle size consistency in order to quickly dissolve in liquids. The agglomeration process starts with the spray dryer process by delivering the fine Colostrum powder back into the liquid Colostrum coming through the spray dryer nozzle. The liquid is delivered into the drying chamber through the nozzle which creates first pass of powder. Then those fines are collected in the baghouse chamber and delivered back into spray stream until the particle size gets large enough to drop into the Fluid Bed (FB). The FB is then used to reduce moisture and add any coatings needed, e.g., lecithin, MCT, etc. The 1× is the typical amount of agglomeration.

TABLE 2
Super Agglomerate Colostrum Compositions

| CODE | | colostrum |
|---|---|---|
| ID | Prototype | wt % |
| A2 | Super agglomerate—lecithin/MCT 1X | ND |
| A3 | Super agglomerate—lecithin/MCT 2X | ND |
| A4 | Super agglomerate—lecithin/MCT 3X | ND |
| A6 | Super agglomerate—colostrum fat 1X | ND |
| A7 | Super agglomerate—colostrum fat 2X | ND |
| A8 | Super agglomerate—colostrum fat 3X | ND |

ND = not determined

The B1-B21 prototypes shown in Table 3 were made by suspending powdered colostrum in a fluid bed apparatus where they were sprayed with a commercially available coating material to a certain percentage by weight. Selected B prototypes were screened and particle sizes were determined. B prototypes B5, B10, B16, and B21 had particle size in the range from 149-594 um diameter.

TABLE 3
Microencapsulated Colostrum Compositions

| CODE | | colostrum |
|---|---|---|
| ID | Prototype | wt % |
| B1 | Microencapsulation—Nutrateric 10% | 90 |
| B2 | Microencapsulation—Nutrateric 20% | 80 |
| B3 | Microencapsulation—Nutrateric 30% | 70 |
| B4 | Microencapsulation—Nutrateric 40% | 60 |
| B5 | Microencapsulation—Nutrateric 50%— | 50 |
| | screened—149-594 um | |
| B6 | Microencapsulation—Protect EN 10% | 90 |
| B7 | Microencapsulation—Protect EN 20% | 80 |
| B8 | Microencapsulation—Protect EN 30% | 70 |
| B9 | Microencapsulation—Protect EN 40% | 60 |
| B10 | Microencapsulation—Protect EN 50%— | 50 |
| | screened—149-594 um | |
| B12 | Microencapsulation—Eudraguard 10% | 90 |
| B13 | Microencapsulation—Eudraguard 20% | 80 |
| B14 | Microencapsulation—Eudraguard 30% | 70 |
| B15 | Microencapsulation—Eudraguard 40% | 60 |
| B16 | Microencapsulation—Eudraguard 50%— | 50 |
| | screened—149-594 um | |
| B17 | Microencapsulation—Stearine 17-A005 10% | 90 |
| B18 | Microencapsulation—Stearine 17-A005 20% | 80 |
| B19 | Microencapsulation—Stearine 17-A005 30% | 70 |
| B20 | Microencapsulation—Stearine 17-A005 40% | 60 |
| B21 | Microencapsulation—Stearine 17-A005 50%— | 50 |
| | screened—149-594 um | |

The C1-C13 prototypes shown in Table 4 had additives mixed into the post-homogenized colostrum and then spray dried. There were three different inclusion rates tested for each additive.

TABLE 4
Matrix Stabilization Colostrum Compositions

| CODE | | colostrum |
|---|---|---|
| ID | Prototype | wt % |
| C1 | Matrix Stabilization—Trehalose 10% | 93.6 |
| C2 | Matrix Stabilization—Trehalose 15% | 90.3 |
| C3 | Matrix Stabilization—Trehalose 20% | 86.7 |
| C4 | Matrix Stabilization—Polydextrose 10% | 90 |
| C5 | Matrix Stabilization—Polydextrose 15% | 85 |
| C6 | Matrix Stabilization—Polydextrose 20% | 80 |
| C8 | Matrix Stabilization—Calcium Caseinate 5% | 95 |
| C9 | Matrix Stabilization—Calcium Caseinate 10% | 90 |
| C10 | Matrix Stabilization—Calcium Caseinate 15% | 85 |
| C11 | nutritional serine protease inhibitor— | 95 |
| | enzyme active Soy Flour 5% | |
| C12 | nutritional serine protease inhibitor— | 90 |
| | enzyme active Soy Flour 10% | |
| C13 | nutritional serine protease inhibitor— | 85 |
| | enzyme active Soy Flour 15% | |

The prototype compositions in Tables 2-4 were subjected to a digestion process protocol outlined in FIG. 1A. A numerical heat map of assay results for each prototype composition for growth factor activity assays in vitro in the Proliferation assay (as % undigested control), growth factor structural stability as EGF (ng/ml), and as TGF beta (pg/ml), immune factor activity assays in vitro as E. coli binding (Absorbance at A450 nm), and immune factor structural stability as IgG (ug/ml) and lactoferrin (ng/ml) is shown in Table 9 and Table 10. FIG. 1D and FIG. 1E, as well as FIG. 2A through FIG. 2D show graphical representations of select compositions. For each composition in FIG. 1D through FIG. 2D, the recited combination product is included at 10%. Thus, “BC+Stearine” would correspond to BC+10% stearine.

Trehalose, stearine, casein and soy flower were each assessed for their ability to influence in vitro stability of BC. As shown in FIG. 1D, exposure of BC alone to HCl/pepsin reduced proliferative bioactivity of the BC as measured by the Alamar blue assay, and proliferation was further reduced following CT exposure. The combination of BC with trehalose, stearine, or casein did not preserve proliferative bioactivity of BC against HCl/pepsin or CT. However, the addition of soy flour did preserve the proliferative activity of BC against both HCl/pepsin and CT digestion. In a further assessment of BC stability, bovine IgG E. coli binding was assessed. As shown in FIG. 1E, exposure of BC alone to HCl/pepsin reduced the IgG:E. coli binding as measured via ELISA assay, and binding was further reduced following CT exposure. The combination of BC with stearine did not affect HCl/pepsin digestion, but did enhance stability against CT digestion. The combination of BC with trehalose had no beneficial effects against HCl/pepsin or CT. However, combination of BC with either casein or soya significantly enhanced stability against both HCl/pepsin and CT digestion.

Trehalose, stearine, casein and soy flower were each further assessed of their ability to influence growth factor and IgG immunoreactivity as a measure of their influence on the in vitro stability of BC. IgG immunoreactivity and all growth factor immunoreactivity were measured via ELISA. As shown in FIG. 2A, exposure of BC alone to HCl/pepsin reduced IgG immunoreactivity, and immunoreactivity was further reduced in the presence of CT. Combination of BC and trehalose had no effect. Combination of BC with casein blunted the loss of immunoreactivity due to HCl/pepsin, but had no effect on the loss of immunoreactivity due to CT. Presence of stearine partially reduced both HCl/pepsin and CT mediated loss of immunoreactivity. Combination of BC with soy-flour was the most beneficial in preserving IgG immunoreactivity against both HCl/pepsin and CT.

With respect to growth factors, FIG. 2B shows that BC alone exposed to HCl/pepsion caused a loss of TGFβ immunoreactivity, and that TGFβ immunoreactivity was completely lost when BC alone was exposed to CT. Stearine, trehalos and casein all enhanced TGFβ immunoreactivity stability against HCl/pepsin, but had no effect against CT. In contrast, BC in the presence of soy flour maintained >90% of its immunoreactivity following HCl/pepsin and CT exposure. FIG. 2C shows that BC alone exposed to HCl/pepsin caused a loss of EGF immunoreactivity, and exposure to CT caused a further reduction in EGF immunoreactivity. Stearine partially preserved BC against CT exposure, but had little to no effect against HCl/pepsin exposure. Both trehalose and casein were ineffective against both HCl/pepsin and CT. However, soy flour was highly effective in preserving activity with EGF immunoreactivity upon both HCl/pepsin and CT exposure. FIG. 2D shows that BC alone exposed to HCl/pepsin caused a loss of lactoferrin immunoreactivity, and exposure to CT caused a further reduction in lactoferrin immunoreactivity. Stearine was ineffective in reducing loss of immunoreactivity whereas trehalose and casein truncated the loss of immunoreactivity following CT exposure. However, neither were effective in preserving immunoreactivity following CT exposure. Co-packaging the BC with soy flour enhanced stability against both HCl/pepsin and CT exposure.

The compositions of Table 2 through Table 4 were also tested in various assays as a measure of in vitro biological activity and structural stability of BC. The results are presented in Table 5A through Table 5C.

TABLE 5A
Numerical heatmap of compositions A2-A8 and B1-B8. Biological activity and
structural stability of growth factors and immune factors in digested samples in vitro.

| | Composition | | | | | | | | | | | | | | |
|---|---|---|---|---|---|---|---|---|---|---|---|---|---|---|---|
| Activity | A1 | A2 | A3 | A4 | A6 | A7 | A8 | B1 | B2 | B3 | B4 | B5 | B6 | B7 | B8 |
| | Growth Factor Activity (in vitro) | | | | | | | | | | | | | | |
| Proliferation | 33 | 1 | 0 | 9 | 0 | 9 | 0 | 21 | 0 | 13 | 0 | 14 | 25 | 26 | 0 |
| (% undigest | | | | | | | | | | | | | | | |
| control) | | | | | | | | | | | | | | | |
| | Growth Factor Stability | | | | | | | | | | | | | | |
| EGF (ng/ml) | 0.5 | 1.0 | 1.0 | 0.7 | 0.2 | 0.6 | 0.0 | 1.1 | 1.2 | 0.9 | 0.2 | 0.5 | 1.1 | 1.8 | 0.8 |
| TGFβ | 0 | 5 | 0 | 8 | 23 | 25 | 0 | 0 | 0 | 0 | 0 | 0 | 0 | 0 | 0 |
| (pg/ml) | | | | | | | | | | | | | | | |
| | Immune Factor Activity (in vitro) | | | | | | | | | | | | | | |
| E. coli | 0.1 | 0.1 | 0.1 | 0.1 | 0.2 | 0.2 | 0.2 | 0.0 | 0.1 | 0.1 | 0.0 | 0.0 | 0.1 | 0.1 | 0.1 |
| binding | | | | | | | | | | | | | | | |
| (A450 nm) | | | | | | | | | | | | | | | |
| | Immune Factor Stability | | | | | | | | | | | | | | |
| IgG (μg/ml) | 7.2 | 5.5 | 6.9 | 6.1 | 7.0 | 7.1 | 7.8 | 4.4 | 4.3 | 5.1 | 1.9 | 1.6 | 5.8 | 4.5 | 5.2 |
| Lactoferrin | 2.2 | 1.9 | 2.4 | 2.2 | 2.1 | 2.3 | 2.7 | 1.8 | 2.2 | 2.3 | 1.6 | 1.0 | 2.2 | 2.2 | 2.3 |
| (ng/ml) | | | | | | | | | | | | | | | |

TABLE 5B
Numerical heatmap of compositions B9-B21. Biological activity and structural stability
of growth factors and immune factors in digested samples in vitro.

| | Composition | | | | | | | | | | | | |
|---|---|---|---|---|---|---|---|---|---|---|---|---|---|
| Activity | A1 | B9 | B10 | B12 | B13 | B14 | B15 | B16 | B17 | B18 | B19 | B20 | B21 |
| | Growth Factor Activity (in vitro) | | | | | | | | | | | | |
| Proliferation | 33 | 28 | 32 | 17 | 18 | 5 | 18 | 9 | 141 | 142 | 22 | 35 | 39 |
| (% undigest | | | | | | | | | | | | | |
| control) | | | | | | | | | | | | | |
| | Growth Factor Stability | | | | | | | | | | | | |
| EGF (ng/ml) | 0.5 | 0.2 | 0.0 | 0.3 | 0.7 | 0.0 | 1.6 | 0.0 | 7.5 | 4.4 | 0.6 | 0.0 | 0.7 |
| TGFβ | 0 | 0 | 0 | 0 | 0 | 0 | 0 | 0 | 0 | 0 | 0 | 0 | 0 |
| (pg/ml) | | | | | | | | | | | | | |
| | Immune Factor Activity (in vitro) | | | | | | | | | | | | |
| E. coli | 0.1 | 0.1 | 0.0 | 0.0 | 0.0 | 0.0 | 0.1 | 0.0 | 0.2 | 0.1 | 0.1 | 0.0 | 0.0 |
| binding | | | | | | | | | | | | | |
| (A450 nm) | | | | | | | | | | | | | |
| | Immune Factor Stability | | | | | | | | | | | | |
| IgG (μg/ml) | 7.2 | 5.3 | 3.4 | 3.6 | 3.3 | 3.7 | 2.3 | 2.7 | 8.3 | 7.9 | 1.9 | 0.9 | 1.7 |
| Lactoferrin | 2.2 | 1.4 | 1.7 | 1.4 | 1.0 | 0.2 | 0.2 | 0.0 | 2.1 | 1.5 | 0.9 | 0.5 | 0.5 |
| (ng/ml) | | | | | | | | | | | | | |

TABLE 5C
Numerical heatmap of compositions C1-C13. Biological activity and structural stability
of growth factors and immune factors in digested samples in vitro.

| | Composition | | | | | | | | | | | | |
|---|---|---|---|---|---|---|---|---|---|---|---|---|---|
| Activity | A1 | C1 | C2 | C3 | C4 | C5 | C6 | C8 | C9 | C10 | C11 | C12 | C13 |
| | Growth Factor Activity (in vitro) | | | | | | | | | | | | |
| Proliferation | 33 | 6 | 9 | 12 | 12 | 46 | 55 | 57 | 41 | 23 | 78 | 72 | 73 |
| (% undigest | | | | | | | | | | | | | |
| control) | | | | | | | | | | | | | |
| | Growth Factor Stability | | | | | | | | | | | | |
| EGF (ng/ml) | 0.5 | 1.2 | 1.6 | 1.6 | 1.4 | 2.6 | 3.7 | 2.3 | 1.4 | 1.0 | 1.6 | 10.8 | 1.1 |
| TGFβ | 0 | 18 | 21 | 28 | 0 | 4 | 0 | 0 | 0 | 0 | 15 | 490 | 130 |
| (pg/ml) | | | | | | | | | | | | | |
| | Immune Factor Activity (in vitro) | | | | | | | | | | | | |
| E. coli | 0.1 | 0.2 | 0.2 | 0.2 | 0.2 | 0.3 | 0.1 | 0.2 | 0.2 | 0.2 | 0.1 | 0.2 | 0.1 |
| binding | | | | | | | | | | | | | |
| (A450 nm) | | | | | | | | | | | | | |
| | Immune Factor Stability | | | | | | | | | | | | |
| IgG (μg/ml) | 7.2 | 8.4 | 6.2 | 3.2 | 10.2 | 8.6 | 6.2 | 10.2 | 8.6 | 7.1 | 8.0 | 11.7 | 4.6 |
| Lactoferrin | 2.2 | 2.2 | 1.8 | 1.7 | 1.8 | 2.7 | 1.5 | 3.0 | 2.3 | 1.9 | 1.7 | 2.5 | 1.7 |
| (ng/ml) | | | | | | | | | | | | | |

In the growth factor activity assays in vitro, improved proliferation as % undigested control when compared to Control Composition A1, was exhibited by prototype compositions B17, B18, B20, and B21 (17 Stearine, hydrogenated soybean oil, 10%, 20%, 40%, 50%), C5, C6 (polydextrose 15% and 20%), C8, C9 (calcium caseinate, 5% and 10%), and C11, C12, and C13 (enzyme active soy flour 5%, 10%, 15%).

In the growth factor structural stability assays, increased EGF (mg/ml) stability compared to Control colostrum A1 was exhibited by prototype compositions A2, A3, A4 (lecithin/MCT super agglomerate 1×, 2×, 3×), A7 (colostrum fat super agglomerate 2×), B1, B2, B3 (Nutrateric®, alginate plus ethyl cellulose aqueous dispersion 10%, 20%, 30%), B6, B7, B8 (Protect EN™, alginate plus shellac, 10%, 20%, 30%), B13, B15 (Eudraguard™, alginate plus maize starch, 20%, 40%), B17, B18, B19, B21 (Stearine 17, hydrogenated vegetable oil 10%, 20%, 30%, 50%), C1, C2, C3 (trehalose, 10%, 15%, 20%), C4, C5, C6 (polydextrose, 10%, 15%, 20%), C8, C9, C10 (calcium caseinate, 5%, 10%, 15%), and C11, C12, C13 (enzyme active soy flour, 5%, 10%, 15%).

In the growth factor structural stability assays, increased TGF beta (mg/ml) stability compared to Control colostrum A1, was exhibited by prototype compositions A2, A4 (lecithin/MCT super agglomerate 1×, 3×), A6, A7 (colostrum fat super agglomerate, 1×, 2×), C1, C2, C3 (trehalose, 10%, 15%, 20%), C5 (polydextrose, 15%), C11, C12, C13 (enzyme active soy flour, 5%, 10%, 15%).

In the immune factor activity in vitro assays, improved E. coli binding (A450 nm) compared to control colostrum A1, was exhibited by prototype compositions A6, A7, A8 (colostrum fat super agglomerate, 1×, 2×, 3×), B17 (stearine microencapsulation, 10%), C1, C2, C3 (trehalose matrix stabilization, 10%, 15%, 20%), C4, C5 (polydextrose matrix stabilization, 10%, 15%), C8, C9, C10 (calcium caseinate matrix stabilization, 5%, 10%, 15%), and C12 (enzyme active soy flour 10%).

In the immune factor structural stability assays, increased IgG (ug/ml) structural stability compared to Control colostrum A1, was exhibited by prototype compositions A8 (colostrum fat super agglomerate, 3×), B17, B18 (stearine microencapsulation, 10%, 20%), C1 (trehalose matrix stabilization, 10%), C4, C5 (polydextrose matrix stabilization, 10%, 15%), C8, C9 (calcium caseinate matrix stabilization, 5%, 10%), C11, C12 (enzyme active soy flour, 5%, 10%).

In the factor structural stability assays, increased lactoferrin (ug/ml) structural stability compared to Control colostrum A1, was exhibited by prototype compositions A3 (lecithin/MCT super agglomerate), A8 (colostrum fat super agglomerate 3×), B3 (Nutrateric, alginate plus ethyl cellulose aqueous dispersion 30%), B8 (Protect EN™ microencapsulation, alginate plus shellac, 30%), C5 (polydextrose matrix stabilization, 15%), C8, C9 (calcium caseinate, 5%, 10%), and C12 (enzyme active soy flour, 10%).

### Example 5. Acid Resistant Capsules Protect Against Acid/Pepsin Digestion

Whole colostrum alone, colostrum+egg, and egg powder alone as described above were encapsulated with either an acid resistant capsule (bioVXR acid resistant HPMC capsules, BioCaps, El Monte, Calif.) or conventional capsules (bovine gelatin). The encapsulated compositions were subjected to a digestion process protocol outlined in FIG. 1A. Briefly, the sample was subjected to 1 mg/mL acid/pepsin at 37° C., 1 hour in rotary incubator. Sodium hydroxide is added to neutralize sample and half of the sample set aside (P). The other half of the (P) sample is subjected to 1 mg/ml chymotrypsin/trypsin digestion at 37° C., 1 h in rotary incubator. This sample is labeled CT). Each of the samples (P) and (CT) are compared to undigested sample (U) in an in vitro proliferation assay using AGS cells, as detailed herein above.

As shown in FIG. 8, acid resistant capsules improved delivery to the small intestine of colostrum, colostrum+egg and egg powder alone (i.e., survival post peptic digestion of about 90% versus about 50% in comparative non-acid resistant conventional capsules), this delivered more to the trypsin chymotrypsin stage so the amount left after CT digestion was about 3× higher than in normal non-acid resistant capsules. Therefore, the acid resistant capsule delivered more intact colostrum to the intestinal digestion stage so more was left, as shown by improved proliferation (%) activity in AGS cells when compared to conventional non-acid resistant capsules.

### Example 6. Trypsin Inhibitor Assay

All samples were assessed for trypsin inhibitor assay using a standard Na-benzoyl-DL-arginine-p-nitroanilide (BAPNA) trypsin inhibitor assay based on method of Erlander et al.

Preparation of compositions comprising colostrum and enzyme active soy flour is shown in Table 5. The trypsin inhibitor (TI) activity of each material was investigated. Specifically, the pro-reparative factor colostrum (C7), the nutritional serine protease inhibitor enzyme active full fat soy flour (C17), and the stabilizing agent calcium caseinate (C16) were assayed for TI activity individually, and in various combinations of the soy flour and colostrum.

Trypsin inhibitor activity was assessed using a standard Na-benzoyl-DL-arginine-p-nitroanilide (BAPNA) trypsin inhibition assay based on the method of Erlander et al., “The Preparation and Properties of Two New Chromogenic Substrates of Trypsin,” Archives of Biochemistry and Biophysics, 95: 2: Pages 271-278 (1961). The assay was carried out by mixing 170 μl of sample (diluted in assay buffer, 50 mM Tris-HCl, pH 8, 20 mM CaCl₂)), with 20 μg of trypsin (made up in 100 mM HCl, T9253, Merck Life Sciences) and adding 1 mL of 0.3 mg/mL BAPNA (dissolved in DMSO and diluted in assay buffer pre warmed to 37° C., B4875, Merck Life Sciences). Samples were incubated at 37° C. for 10 minutes and the reaction was stopped by adding 143 μl 30% acetic acid. The formation of p-nitroaniline was measured at 405 nm with a microplate reader. A blank control was prepared by for each sample by adding 100 mM HCl instead of trypsin.

Trypsin inhibitor activity of three different lots of enzyme active full fat soy flour (Easy 100, Natural Products, Inc., Grinnell, Iowa) S1, S2, and S3 was also determined. Three mixtures of colostrum/soy flour were including 5 wt % soy flour/95 wt % colostrum (C11), 10 wt % soy flour/90 wt % colostrum (C12), and 15 wt % soy flour/85 wt % colostrum (C13), were prepared as shown in Table 6 and spray dried prior to TI assay. The % solids of the liquid colostrum was measured and amount of soy to add was calculated based on percentage of total solids (colostrum plus soy). So, e.g., C12 (run 11) had 800 total grams of solids with ˜80 g being the soy flour, and 720 g being the colostrum. Each run size used 4.0 kg liquid colostrum, with water 82%, 18% solids, and solids 0.72 kg.

TABLE 6
Compositions comprising colostrum + enzyme active soy flour

| | | | | | Total | |
|---|---|---|---|---|---|---|
| | | | | | theoretical | |
| | | | | Material | solids | |
| | | Mix in | | to | (colo + | |
| | | (wt % | Colostrum | mix in | mix | % |
| Run | Material | solids) | solds (kg) | (grams) | in) kgs | check |
| Run | EASY100 | 5.0% | 0.720 | 37.80 | 0.758 | 5.0% |
| 10 | soy flour | | | | | |
| (C11) | | | | | | |
| Run | EASY100 | 10.0% | 0.720 | 79.92 | 0.800 | 10.0% |
| 11 | soy flour | | | | | |
| (C12) | | | | | | |
| Run | EASY100 | 15.0% | 0.720 | 127.44 | 0.847 | 15.0% |
| 12 | soy flour | | | | | |
| (C13) | | | | | | |

Results of the trypsin inhibitor assay are shown in Table 7. Samples were analyzed in duplicate with appropriate blanks. Dose response curves of the samples showed linearity of results.

TABLE 7
Results of Trypsin Inhibitor Assay

| | μg trypsin | Theoretical TI | % variation |
|---|---|---|---|
| | inhibited by | from individual | from |
| Sample | 1 mg sample | constituents | theoretical |
| C7—colostrum | 7.41 +/− 0.09 | — | — |
| C11—5% matrix | 11.50 +/− 1.29 | 7.039 + 2.148 = | 25% |
| stabilization soy flour | | 9.188 | |
| C12—10% matrix | 15.48 +/− 0.85 | 6.669 + 4.296 = | 41% |
| stabilization soy flour | | 10.965 | |
| C13—15% matrix | 17.99 +/− 0.86 | 6.299 + 6.152 = | 25% |
| stabilization soy flour | | 14.451 | |
| C16—casein caseinate | 3.24 +/− 0.29 | — | — |
| Cl7—soy flour | 42.96 +/− 0.69 | — | — |
| S1—Enzyme active full | 41.01 +/− 1.46 | — | — |
| fat soy flour (Easy 100) | | | |
| Lot 072720 | | | |
| S2—Enzyme active full fat | 39.71 +/− 2.01 | — | — |
| soy flour (Easy 100) Lot | | | |
| 082720-2 | | | |
| S3—Enzyme active full fat | 40.95 +/− 1.72 | — | |
| soy flour (Easy 100) Lot | | | |
| 061820-2 | | | |

Theoretical results were derived from the expected contributions of the subcomponents added in isolation. As can be seen in Table 7, an unexpected synergistic response of about 25% expected TI activity was seen when soya and colostrum were combined suggesting the two components work in a complimentary way to enhance the trypsin inhibitory activity.

The combination products stearine, trehalose, casein and soy flour were also assessed for trypsin inhibitor activity based on the methods of Erlander as described above. The results are shown in Table 8.

TABLE 8
Trypsin inhibitor activity of combination products
tested alone and in combination.

| | | Theoretical | % |
|---|---|---|---|
| | | TI from | variation |
| | | individual | from |
| Sample | μg trypsin inhibited | constituents | theoretical |
| BC (1 mg/ml) | 7.55 +/− 0.33 | 7.55 | 0 |
| BC (0.9 mg/ml) | 6.87 +/− 0.30 | 6.87 | 0 |
| Stearine (0.1 mg/ml) | 0.05 +/− 0.05 | 0.05 | 0 |
| Trehalose (0.1 | 0.02 +/− 0.03 | 0.02 | 0 |
| mg/ml) | | | |
| Casein (0.1 mg/ml) | 0.34 +/− 0.09 | 0.34 | 0 |
| Soy flour (0.1 mg/ml) | 4.11 +/− 0.25 | 4.11 | 0 |
| BC (0.9 mg/ml) + | 7.52 +/− 0.38 | 6.87 + 0.05 = | 3.9% |
| stearine (0.1 mg/ml) | | 6.92 | |
| BC (0.9 mg/ml) + | 7.35 +/− 0.34 | 6.87 + 0.02 = | 6.2% |
| trehalose (0.1 mg/ml) | | 6.89 | |
| BC (0.9 mg/ml) + | 8.66 +/− 0.09 | 6.87 + 0.34 = | 20.7% |
| casein (0.1 mg/ml) | | 7.21 | |
| BC (0.9 mg/ml) + soy | 16.03 +/− 0.57 | 6.87 + 4.11 = | 57.1% |
| flour (0.1 mg/ml) | | 10.98 | |

As can be seen in Table 8, stearine and trehalose had negligible amounts of TI activity when tested alone. On a per mg basis, soy flour had 12 times the TI activity versus casein and 5 times the activity of BC when each component was tested in isolation. Surprisingly, an unexpected synergistic response of about 21% and about 57% expected TI activity was seen when BC was combined with casein or soy flour respectively, suggesting the two components work in a complimentary way to enhance the trypsin inhibitory activity.

Trypsin inhibitor activity of bovine colostrum (BC), egg, BC+egg, ovomucoid and SBTI samples was assessed using the standard Na-benzoyl-DL-arginine-p-nitroanilide (BAPNA) trypsin inhibition assay based on the method of Erlander et al. Results are shown in Table 9. Samples were analyzed in duplicate with appropriate blanks. Dose response curves of the samples showed linearity of results. Results expressed as mean+/−sem.

TABLE 9
Further Results of Trypsin Inhibitor Assay

| Sample | μg trypsin inhibited by 1 mg sample |
|---|---|
| BC | 7.41 +/− 0.44 |
| Egg | 42.93 +/− 4.41 |
| BC + Egg | 19.44 +/− 0.99 |
| EGF | 1.43 +/− 0.12 |
| Ovomucoid | 702.54 +/− 7.52 |
| SBTI | 838.0 +/− 8.94 |

As shown in Table 9, the nutritional serine protease inhibitors Ovomucoid and SBTI possessed strong trypsin inhibitor activity, the egg and BC intermediate activity, with EGF possessing virtually no trypsin inhibitory activity.

What is claimed is:

1. A composition comprising a pro-reparative factor, a nutritional protease inhibitor, and optionally a nutraceutically acceptable carrier or excipient.

2. The composition of claim 1, wherein the composition further comprises a stabilizing agent.

3. The composition of claim 2, wherein the stabilizing agent is casein, caseinate salt, calcium caseinate, sodium caseinate, caseinate isolate from colostrum, polydextrose, trehalose, exogenous colostrum fat, stearin, alginate, alginate plus ethyl cellulose aqueous dispersion, alginate plus ethyl cellulose, alginate plus maize starch, alginate plus acrylic polymer, or a combination thereof.

4. The composition of claim 1, wherein the pro-reparative factor is selected from a colostrum, late colostrum, skim colostrum, partially defatted colostrum, a mixture of colostrum whey concentrate and whey protein concentrate, whey protein concentrate, an egg product, an isolated pro-reparative peptide or protein, a nutraceutically acceptable salt thereof, or a combination thereof.

5. The composition of claim 4, wherein the colostrum is a bovine colostrum.

6. The composition of claim 5, wherein the bovine colostrum is selected from skim bovine colostrum, partially defatted bovine colostrum, or whole bovine colostrum.

7. The composition of claim 4, wherein the isolated pro-reparative peptide or protein is a recombinant pro-reparative peptide or protein comprising Epidermal Growth Factor (EGF), Transforming Growth Factor α (TGF-α), Transforming Growth Factor β (TGF-β), Insulin-Like Growth Factor I (IGF-I), Insulin-Like Growth Factor II (IGF-II) Platelet-derived growth factor (PDGF), Vascular Endothelial Growth Factor (VEGF), Milk fat globule-epidermal growth factor 8 (MFG-E8), or nutraceutically acceptable salts thereof.

8. The composition of claim 1, wherein the nutritional protease inhibitor is casein or a nutritional trypsin inhibitor.

9. (canceled)

10. The composition of claim 8, wherein the nutritional trypsin inhibitor is an enzyme active soy product, an ovomucoid, a soybean trypsin inhibitor, or a combination thereof.

11. The composition of claim 10, wherein the enzyme active soy product is raw soy flour, raw soy meal, raw soy product, minimally processed soybean product, or a combination thereof.

12. The composition of claim 11, wherein the enzyme active soy product is an enzyme active soy flour.

13. The composition of claim 12, comprising a weight ratio of colostrum, late colostrum, skim colostrum, partially defatted colostrum, or mixture of colostrum whey concentrate and whey protein concentrate to enzyme active soy product from about 1:1 to about 20:1, optionally from about 5:1 to about 15:1.

14. The composition of claim 1 comprising:
about 40 wt % to about 95 wt % colostrum, late colostrum, skim colostrum, partially defatted colostrum, or a mixture of colostrum whey concentrate and whey protein concentrate; and
about 5 wt % to about 25 wt % enzyme active soy product.

15-17. (canceled)

18. The composition of claim 14, further comprising soy lectin, medium-chain triglycerides, whey protein concentrate, non-fat dry milk, milk protein concentrate, flavoring, or combinations thereof.

19-21. (canceled)

22. The composition of claim 14, wherein the composition comprises a total protein content from about 50 wt % to about 80 wt % of the composition.

23. The composition of claim 14, wherein the composition comprises immunoglobulin G in an amount from about 15 wt % to about 60 wt % of the composition.

24-42. (canceled)

43. A method of treating, alleviating, or preventing a disease or disorder associated with inflammation of a gastrointestinal tract in a subject in need thereof, comprising administering a composition to the subject, the composition comprising:
a) a therapeutically effective amount of a pro-reparative factor, an immune factor, or a combination thereof;
b) a nutritional protease inhibitor; and
c) optionally a nutraceutically acceptable carrier or excipient.

44. The method of claim 43, wherein the composition further comprises a stabilizing agent.

45. (canceled)

46. The method of claim 43, wherein the pro-reparative factor is selected from a colostrum, late colostrum, skim colostrum, partially defatted colostrum, a mixture of colostrum whey concentrate and whey protein concentrate, an egg product, an isolated pro-reparative peptide or protein, or a nutraceutically acceptable salt thereof, or a combination thereof.

47-48. (canceled)

49. The method of claim 46, wherein the isolated pro-reparative peptide or protein is a recombinant pro-reparative peptide or protein comprising Epidermal Growth Factor (EGF), Transforming Growth Factor α (TGF-α), Transforming Growth Factor β (TGF-β), Insulin-Like Growth Factor I (IGF-I), and Insulin-Like Growth Factor II (IGF-II), Platelet-derived growth factor (PDGF), Vascular Endothelial Growth Factor (VEGF), Milk fat globule-epidermal growth factor 8 (MFG-E8), or nutraceutically acceptable salts thereof.

50-85. (canceled)

86. A composition comprising a purified lactoferrin, a stabilizing agent, and optionally a nutraceutically acceptable carrier or excipient.

87. (canceled)
